SEQUENCE LISTING (89 sequences)

SEQ ID NO: 1 - protein, 203 aa, synthetic construct
  VARIANT 1: note = optionally absent
MEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 2 - protein, 204 aa, synthetic construct
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 3 - protein, 204 aa, synthetic construct
RFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 4 - protein, 204 aa, synthetic construct
RFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKST

SEQ ID NO: 5 - protein, 204 aa, synthetic construct
RFPNITNLCPFDEVFNATRFASVYAWNRKRISNCVADFSVLYNFAPFFAFKCYGVSPTKLNDLCWTNIYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKST

SEQ ID NO: 6 - protein, 204 aa, synthetic construct
RFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKST

SEQ ID NO: 7 - protein, 204 aa, synthetic construct
RFPNITNLCPFDEVFNATRFASVYAWNRKRISNCVADFSVLYNLAPFFTFKCYGVSPTKLNDLCWTNIYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKST

SEQ ID NO: 8 - protein, 204 aa, synthetic construct
RFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKST

SEQ ID NO: 9 - protein, 204 aa, synthetic construct
RFPNITNLCPFDEVFNATRFASVYAWNRKRISNCVADFSVLYNFAPFFAFKCYGVSPTKLNDLCWTNIYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKST

SEQ ID NO: 10 - protein, 204 aa, synthetic construct
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKST

SEQ ID NO: 11 - protein, 204 aa, synthetic construct
RFPNITNLCPFGEVFNATTFASVYAWNRKRISNCVADYSVLYNSTSFSTFKCYGVSPTKLNDLCFTNVYADSFVITGDEVRQIAPGQTGKIADYNYKLPDDFTGCVIAWNSKHIDAKEGGNFNYLYRLFRKANLKPFERDISTEIYQAGSKPCNGQTGLNCYYPLYRYGFYPTDGVGHQPYRVVVLSFELLNAPATVCGPKKST

SEQ ID NO: 12 - protein, 202 aa, synthetic construct
RFPNITNLCPFGEVFNASKFASVYAWNRKRISNCVADYSVLYNSTSFSTFKCYGVSPTKLNDLCFTNVYADSFVVKGDEVRQIAPGQTGVIADYNYKLPDDFTGCVIAWNSVKQDALTGGNYLYRLFRKSKLKPFERDISTEIYQAGSTPCNGQVGLNCYYPLERYGFHPTTGVNYQPFRVVVLSFELLNGPATVCGPKLST

SEQ ID NO: 13 - protein, 203 aa, synthetic construct
RFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLST

SEQ ID NO: 14 - protein, 203 aa, synthetic construct
RFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPKLST

SEQ ID NO: 15 - protein, 203 aa, synthetic construct
RFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFTGCVLAWNTRNIDATQTGNYNYKYRSLRHGKLRPFERDISNVPFSPDGKPCTPPAFNCYWPLNDYGFYITNGIGYQPYRVVVLSFELLNAPATVCGPKLST

SEQ ID NO: 16 - protein, 203 aa, synthetic construct
RFPNITNLCPFGQVFNASNFPSVYAWERLRISDCVADYAVLYNSSSSFSTFKCYGVSPTKLNDLCFSSVYADYFVVKGDDVRQIAPAQTGVIADYNYKLPDDFTGCVLAWNTNSVDSKSGNNFYYRLFRHGKIKPYERDISNVLYNSAGGTCSSISQLGCYEPLKSYGFTPTVGVGYQPYRVVVLSFELLNAPATVCGPKKST

SEQ ID NO: 17 - protein, 203 aa, synthetic construct
RFPNITNLCPFGQVFNASNFPSVYAWERLRISDCVADFAVLYNSSSSFSTFKCYGVSPTKLNDLCWSSIYADYFVVKGDDVRQIAPAQTGVIADYNYKLPDDFTGCVLAWNTNSVDSKSGNNFYYRLFRHGKIKPYERDISNVLYNSAGGTCSSISQLGCYEPLKSYGFTPTVGVGYQPYRVVVLSFELLNAPATVCGPKKST

SEQ ID NO: 18 - protein, 203 aa, synthetic construct
RFPNITNLCPFGQVFNASKFPSVYAWERLRISDCVADYSVLYNSSSSFSTFKCYGVSPTKLNDLCFSSVYADYFVVKGDDVRQIAPAQTGVIADYNYKLPDDFTGCVLAWNTNSVDSKQGNNFYYRLFRHGKIKPYERDISNVLYNSAGGTCSSTSQLGCYEPLKSYGFTPTVGVGYQPYRVVVLSFELLNAPATVCGPKKST

SEQ ID NO: 19 - protein, 200 aa, synthetic construct
RFPNITQLCPFNEVFNITSFPSVYAWERMRITNCVADYSVLYNSSASFSTFQCYGVSPTKLNDLCFSSVYADYFVVKGDDVRQIAPAQTGVIADYNYKLPDDFTGCVIAWNTNSLDSSNEFFYRRFRHGKIKPYGRDLSNVLFNPSGGTCSAEGLNCYKPLASYGFTQSSGIGFQPYRVVVLSFELLNAPATVCGPKQST

SEQ ID NO: 20 - protein, 436 aa, synthetic construct
  VARIANT 205..232: note = optionally absent
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEC

SEQ ID NO: 21 - protein, 435 aa, synthetic construct
  VARIANT 205..232: note = optionally absent
RFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 22 - protein, 435 aa, synthetic construct
  VARIANT 205..232: note = optionally absent
RFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 23 - protein, 435 aa, synthetic construct
  VARIANT 205..232: note = optionally absent
RFPNITNLCPFDEVFNATRFASVYAWNRKRISNCVADFSVLYNFAPFFAFKCYGVSPTKLNDLCWTNIYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 24 - protein, 435 aa, synthetic construct
  VARIANT 205..232: note = optionally absent
RFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 25 - protein, 435 aa, synthetic construct
  VARIANT 205..232: note = optionally absent
RFPNITNLCPFDEVFNATRFASVYAWNRKRISNCVADFSVLYNLAPFFTFKCYGVSPTKLNDLCWTNIYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 26 - protein, 435 aa, synthetic construct
  VARIANT 205..232: note = optionally absent
RFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 27 - protein, 435 aa, synthetic construct
  VARIANT 205..232: note = optionally absent
RFPNITNLCPFDEVFNATRFASVYAWNRKRISNCVADFSVLYNFAPFFAFKCYGVSPTKLNDLCWTNIYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 28 - protein, 435 aa, synthetic construct
  VARIANT 205..232: note = optionally absent
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 29 - protein, 435 aa, synthetic construct
  VARIANT 205..232: note = optionally absent
RFPNITNLCPFGEVFNATTFASVYAWNRKRISNCVADYSVLYNSTSFSTFKCYGVSPTKLNDLCFTNVYADSFVITGDEVRQIAPGQTGKIADYNYKLPDDFTGCVIAWNSKHIDAKEGGNFNYLYRLFRKANLKPFERDISTEIYQAGSKPCNGQTGLNCYYPLYRYGFYPTDGVGHQPYRVVVLSFELLNAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 30 - protein, 433 aa, synthetic construct
  VARIANT 203..230: note = optionally absent
RFPNITNLCPFGEVFNASKFASVYAWNRKRISNCVADYSVLYNSTSFSTFKCYGVSPTKLNDLCFTNVYADSFVVKGDEVRQIAPGQTGVIADYNYKLPDDFTGCVIAWNSVKQDALTGGNYLYRLFRKSKLKPFERDISTEIYQAGSTPCNGQVGLNCYYPLERYGFHPTTGVNYQPFRVVVLSFELLNGPATVCGPKLSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 31 - protein, 434 aa, synthetic construct
  VARIANT 204..231: note = optionally absent
RFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 32 - protein, 434 aa, synthetic construct
  VARIANT 204..231: note = optionally absent
RFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPKLSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 33 - protein, 434 aa, synthetic construct
  VARIANT 204..231: note = optionally absent
RFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFTGCVLAWNTRNIDATQTGNYNYKYRSLRHGKLRPFERDISNVPFSPDGKPCTPPAFNCYWPLNDYGFYITNGIGYQPYRVVVLSFELLNAPATVCGPKLSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 34 - protein, 434 aa, synthetic construct
  VARIANT 204..231: note = optionally absent
RFPNITNLCPFGQVFNASNFPSVYAWERLRISDCVADYAVLYNSSSSFSTFKCYGVSPTKLNDLCFSSVYADYFVVKGDDVRQIAPAQTGVIADYNYKLPDDFTGCVLAWNTNSVDSKSGNNFYYRLFRHGKIKPYERDISNVLYNSAGGTCSSISQLGCYEPLKSYGFTPTVGVGYQPYRVVVLSFELLNAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 35 - protein, 434 aa, synthetic construct
  VARIANT 204..231: note = optionally absent
RFPNITNLCPFGQVFNASNFPSVYAWERLRISDCVADFAVLYNSSSSFSTFKCYGVSPTKLNDLCWSSIYADYFVVKGDDVRQIAPAQTGVIADYNYKLPDDFTGCVLAWNTNSVDSKSGNNFYYRLFRHGKIKPYERDISNVLYNSAGGTCSSISQLGCYEPLKSYGFTPTVGVGYQPYRVVVLSFELLNAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 36 - protein, 434 aa, synthetic construct
  VARIANT 204..231: note = optionally absent
RFPNITNLCPFGQVFNASKFPSVYAWERLRISDCVADYSVLYNSSSSFSTFKCYGVSPTKLNDLCFSSVYADYFVVKGDDVRQIAPAQTGVIADYNYKLPDDFTGCVLAWNTNSVDSKQGNNFYYRLFRHGKIKPYERDISNVLYNSAGGTCSSTSQLGCYEPLKSYGFTPTVGVGYQPYRVVVLSFELLNAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 37 - protein, 431 aa, synthetic construct
  VARIANT 201..228: note = optionally absent
RFPNITQLCPFNEVFNITSFPSVYAWERMRITNCVADYSVLYNSSASFSTFQCYGVSPTKLNDLCFSSVYADYFVVKGDDVRQIAPAQTGVIADYNYKLPDDFTGCVIAWNTNSLDSSNEFFYRRFRHGKIKPYGRDLSNVLFNPSGGTCSAEGLNCYKPLASYGFTQSSGIGFQPYRVVVLSFELLNAPATVCGPKQSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 38 - protein, 416 aa, synthetic construct
  VARIANT 186..213: note = optionally absent
RFPNITNFCPFDKVFNATRFPNVYAWQRTKISDCIADYTVLYNSTSFSTFKCYGVSPSKLIDLCFTSVYADTFLIRFSEVRQIAPGETGVIADYNYKLPDDFTGCVLAWNTAQQDIGSYFYRSHRAVKLKPFERDLSSDENGVRTLSTYDFNPNVPLDYQATRVVVLSFELLNAPATVCGPKLSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 39 - protein, 399 aa, synthetic construct
  VARIANT 169..196: note = optionally absent
RFPNVYAWERTKISDCVADYTVLYNSTSFSTFKCYGVSPSKLIDLCFTSVYADTFLIRSSEVRQVAPGETGVIADYNYKLPDDFTGCVIAWNTAKHDTGNYYYRSHRKTKLKPFERDLSSDDGNGVYTLSTYDFNPNVPVAYQATRVVVLSFELLNAPATVCGPKLSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 40 - protein, 413 aa, synthetic construct
  VARIANT 183..210: note = optionally absent
RFPSVYAWTRERISNCIADYSVLYNSTSFSTFRCYGVSPTKLNDLCFSNVYADSMVVRGDEVRQIAPSQTGVIADYNYKLPDDFTGCVIAWNSKAKDENGQYFYRLFRKSKLLPFQRDVSNVTYGSGKNDGCNPSEADCYWPLLKYGFTSSVSQDYQPYRVVVLSFELLNAPATVCGPKRSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 41 - protein, 467 aa, synthetic construct
  VARIANT 237..264: note = optionally absent
MGILPSPGMPALLSLVSLLSVLLMGCVAETGTRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 42 - protein, 467 aa, synthetic construct
  VARIANT 237..264: note = optionally absent
MGILPSPGMPALLSLVSLLSVLLMGCVAETGTRFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 43 - protein, 467 aa, synthetic construct
  VARIANT 237..264: note = optionally absent
MGILPSPGMPALLSLVSLLSVLLMGCVAETGTRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 44 - protein, 467 aa, synthetic construct
  VARIANT 237..264: note = optionally absent
MGILPSPGMPALLSLVSLLSVLLMGCVAETGTRFPNITNLCPFDEVFNATRFASVYAWNRKRISNCVADFSVLYNFAPFFAFKCYGVSPTKLNDLCWTNIYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 45 - protein, 467 aa, synthetic construct
  VARIANT 237..264: note = optionally absent
MGILPSPGMPALLSLVSLLSVLLMGCVAETGTRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 46 - protein, 467 aa, synthetic construct
  VARIANT 237..264: note = optionally absent
MGILPSPGMPALLSLVSLLSVLLMGCVAETGTRFPNITNLCPFDEVFNATRFASVYAWNRKRISNCVADFSVLYNLAPFFTFKCYGVSPTKLNDLCWTNIYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 47 - protein, 467 aa, synthetic construct
  VARIANT 237..264: note = optionally absent
MGILPSPGMPALLSLVSLLSVLLMGCVAETGTRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 48 - protein, 467 aa, synthetic construct
  VARIANT 237..264: note = optionally absent
MGILPSPGMPALLSLVSLLSVLLMGCVAETGTRFPNITNLCPFDEVFNATRFASVYAWNRKRISNCVADFSVLYNFAPFFAFKCYGVSPTKLNDLCWTNIYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 49 - protein, 467 aa, synthetic construct
  VARIANT 237..264: note = optionally absent
MGILPSPGMPALLSLVSLLSVLLMGCVAETGTRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 50 - protein, 467 aa, synthetic construct
  VARIANT 237..264: note = optionally absent
MGILPSPGMPALLSLVSLLSVLLMGCVAETGTRFPNITNLCPFGEVFNATTFASVYAWNRKRISNCVADYSVLYNSTSFSTFKCYGVSPTKLNDLCFTNVYADSFVITGDEVRQIAPGQTGKIADYNYKLPDDFTGCVIAWNSKHIDAKEGGNFNYLYRLFRKANLKPFERDISTEIYQAGSKPCNGQTGLNCYYPLYRYGFYPTDGVGHQPYRVVVLSFELLNAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 51 - protein, 465 aa, synthetic construct
  VARIANT 235..262: note = optionally absent
MGILPSPGMPALLSLVSLLSVLLMGCVAETGTRFPNITNLCPFGEVFNASKFASVYAWNRKRISNCVADYSVLYNSTSFSTFKCYGVSPTKLNDLCFTNVYADSFVVKGDEVRQIAPGQTGVIADYNYKLPDDFTGCVIAWNSVKQDALTGGNYLYRLFRKSKLKPFERDISTEIYQAGSTPCNGQVGLNCYYPLERYGFHPTTGVNYQPFRVVVLSFELLNGPATVCGPKLSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 52 - protein, 466 aa, synthetic construct
  VARIANT 236..263: note = optionally absent
MGILPSPGMPALLSLVSLLSVLLMGCVAETGTRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 53 - protein, 466 aa, synthetic construct
  VARIANT 236..263: note = optionally absent
MGILPSPGMPALLSLVSLLSVLLMGCVAETGTRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPKLSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 54 - protein, 466 aa, synthetic construct
  VARIANT 236..263: note = optionally absent
MGILPSPGMPALLSLVSLLSVLLMGCVAETGTRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFTGCVLAWNTRNIDATQTGNYNYKYRSLRHGKLRPFERDISNVPFSPDGKPCTPPAFNCYWPLNDYGFYITNGIGYQPYRVVVLSFELLNAPATVCGPKLSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 55 - protein, 466 aa, synthetic construct
  VARIANT 236..263: note = optionally absent
MGILPSPGMPALLSLVSLLSVLLMGCVAETGTRFPNITNLCPFGQVFNASNFPSVYAWERLRISDCVADYAVLYNSSSSFSTFKCYGVSPTKLNDLCFSSVYADYFVVKGDDVRQIAPAQTGVIADYNYKLPDDFTGCVLAWNTNSVDSKSGNNFYYRLFRHGKIKPYERDISNVLYNSAGGTCSSISQLGCYEPLKSYGFTPTVGVGYQPYRVVVLSFELLNAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 56 - protein, 466 aa, synthetic construct
  VARIANT 236..263: note = optionally absent
MGILPSPGMPALLSLVSLLSVLLMGCVAETGTRFPNITNLCPFGQVFNASNFPSVYAWERLRISDCVADFAVLYNSSSSFSTFKCYGVSPTKLNDLCWSSIYADYFVVKGDDVRQIAPAQTGVIADYNYKLPDDFTGCVLAWNTNSVDSKSGNNFYYRLFRHGKIKPYERDISNVLYNSAGGTCSSISQLGCYEPLKSYGFTPTVGVGYQPYRVVVLSFELLNAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 57 - protein, 466 aa, synthetic construct
  VARIANT 236..263: note = optionally absent
MGILPSPGMPALLSLVSLLSVLLMGCVAETGTRFPNITNLCPFGQVFNASKFPSVYAWERLRISDCVADYSVLYNSSSSFSTFKCYGVSPTKLNDLCFSSVYADYFVVKGDDVRQIAPAQTGVIADYNYKLPDDFTGCVLAWNTNSVDSKQGNNFYYRLFRHGKIKPYERDISNVLYNSAGGTCSSTSQLGCYEPLKSYGFTPTVGVGYQPYRVVVLSFELLNAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 58 - protein, 463 aa, synthetic construct
  VARIANT 233..260: note = optionally absent
MGILPSPGMPALLSLVSLLSVLLMGCVAETGTRFPNITQLCPFNEVFNITSFPSVYAWERMRITNCVADYSVLYNSSASFSTFQCYGVSPTKLNDLCFSSVYADYFVVKGDDVRQIAPAQTGVIADYNYKLPDDFTGCVIAWNTNSLDSSNEFFYRRFRHGKIKPYGRDLSNVLFNPSGGTCSAEGLNCYKPLASYGFTQSSGIGFQPYRVVVLSFELLNAPATVCGPKQSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 59 - protein, 448 aa, synthetic construct
  VARIANT 218..245: note = optionally absent
MGILPSPGMPALLSLVSLLSVLLMGCVAETGTRFPNITNFCPFDKVFNATRFPNVYAWQRTKISDCIADYTVLYNSTSFSTFKCYGVSPSKLIDLCFTSVYADTFLIRFSEVRQIAPGETGVIADYNYKLPDDFTGCVLAWNTAQQDIGSYFYRSHRAVKLKPFERDLSSDENGVRTLSTYDFNPNVPLDYQATRVVVLSFELLNAPATVCGPKLSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 60 - protein, 431 aa, synthetic construct
  VARIANT 201..228: note = optionally absent
MGILPSPGMPALLSLVSLLSVLLMGCVAETGTRFPNVYAWERTKISDCVADYTVLYNSTSFSTFKCYGVSPSKLIDLCFTSVYADTFLIRSSEVRQVAPGETGVIADYNYKLPDDFTGCVIAWNTAKHDTGNYYYRSHRKTKLKPFERDLSSDDGNGVYTLSTYDFNPNVPVAYQATRVVVLSFELLNAPATVCGPKLSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 61 - protein, 445 aa, synthetic construct
  VARIANT 215..242: note = optionally absent
MGILPSPGMPALLSLVSLLSVLLMGCVAETGTRFPSVYAWTRERISNCIADYSVLYNSTSFSTFRCYGVSPTKLNDLCFSNVYADSMVVRGDEVRQIAPSQTGVIADYNYKLPDDFTGCVIAWNSKAKDENGQYFYRLFRKSKLLPFQRDVSNVTYGSGKNDGCNPSEADCYWPLLKYGFTSSVSQDYQPYRVVVLSFELLNAPATVCGPKRSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATE

SEQ ID NO: 62 - protein, 478 aa, synthetic construct
MGILPSPGMPALLSLVSLLSVLLMGCVAETGTRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 63 - protein, 478 aa, synthetic construct
MGILPSPGMPALLSLVSLLSVLLMGCVAETGTRFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 64 - protein, 478 aa, synthetic construct
MGILPSPGMPALLSLVSLLSVLLMGCVAETGTRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 65 - protein, 478 aa, synthetic construct
MGILPSPGMPALLSLVSLLSVLLMGCVAETGTRFPNITNLCPFDEVFNATRFASVYAWNRKRISNCVADFSVLYNFAPFFAFKCYGVSPTKLNDLCWTNIYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 66 - protein, 478 aa, synthetic construct
MGILPSPGMPALLSLVSLLSVLLMGCVAETGTRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 67 - protein, 478 aa, synthetic construct
MGILPSPGMPALLSLVSLLSVLLMGCVAETGTRFPNITNLCPFDEVFNATRFASVYAWNRKRISNCVADFSVLYNLAPFFTFKCYGVSPTKLNDLCWTNIYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 68 - protein, 478 aa, synthetic construct
MGILPSPGMPALLSLVSLLSVLLMGCVAETGTRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 69 - protein, 478 aa, synthetic construct
MGILPSPGMPALLSLVSLLSVLLMGCVAETGTRFPNITNLCPFDEVFNATRFASVYAWNRKRISNCVADFSVLYNFAPFFAFKCYGVSPTKLNDLCWTNIYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 70 - protein, 478 aa, synthetic construct
MGILPSPGMPALLSLVSLLSVLLMGCVAETGTRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 71 - protein, 478 aa, synthetic construct
MGILPSPGMPALLSLVSLLSVLLMGCVAETGTRFPNITNLCPFGEVFNATTFASVYAWNRKRISNCVADYSVLYNSTSFSTFKCYGVSPTKLNDLCFTNVYADSFVITGDEVRQIAPGQTGKIADYNYKLPDDFTGCVIAWNSKHIDAKEGGNFNYLYRLFRKANLKPFERDISTEIYQAGSKPCNGQTGLNCYYPLYRYGFYPTDGVGHQPYRVVVLSFELLNAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 72 - protein, 476 aa, synthetic construct
MGILPSPGMPALLSLVSLLSVLLMGCVAETGTRFPNITNLCPFGEVFNASKFASVYAWNRKRISNCVADYSVLYNSTSFSTFKCYGVSPTKLNDLCFTNVYADSFVVKGDEVRQIAPGQTGVIADYNYKLPDDFTGCVIAWNSVKQDALTGGNYLYRLFRKSKLKPFERDISTEIYQAGSTPCNGQVGLNCYYPLERYGFHPTTGVNYQPFRVVVLSFELLNGPATVCGPKLSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 73 - protein, 477 aa, synthetic construct
MGILPSPGMPALLSLVSLLSVLLMGCVAETGTRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 74 - protein, 477 aa, synthetic construct
MGILPSPGMPALLSLVSLLSVLLMGCVAETGTRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFLGCVLAWNTNSKDSSTSGNYNYLYRWVRRSKLNPYERDLSNDIYSPGGQSCSAVGPNCYNPLRPYGFFTTAGVGHQPYRVVVLSFELLNAPATVCGPKLSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 75 - protein, 477 aa, synthetic construct
MGILPSPGMPALLSLVSLLSVLLMGCVAETGTRFPNITNLCPFGEVFNATTFPSVYAWERKRISNCVADYSVLYNSTSFSTFKCYGVSATKLNDLCFSNVYADSFVVKGDDVRQIAPGQTGVIADYNYKLPDDFTGCVLAWNTRNIDATQTGNYNYKYRSLRHGKLRPFERDISNVPFSPDGKPCTPPAFNCYWPLNDYGFYITNGIGYQPYRVVVLSFELLNAPATVCGPKLSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 76 - protein, 477 aa, synthetic construct
MGILPSPGMPALLSLVSLLSVLLMGCVAETGTRFPNITNLCPFGQVFNASNFPSVYAWERLRISDCVADYAVLYNSSSSFSTFKCYGVSPTKLNDLCFSSVYADYFVVKGDDVRQIAPAQTGVIADYNYKLPDDFTGCVLAWNTNSVDSKSGNNFYYRLFRHGKIKPYERDISNVLYNSAGGTCSSISQLGCYEPLKSYGFTPTVGVGYQPYRVVVLSFELLNAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 77 - protein, 477 aa, synthetic construct
MGILPSPGMPALLSLVSLLSVLLMGCVAETGTRFPNITNLCPFGQVFNASNFPSVYAWERLRISDCVADFAVLYNSSSSFSTFKCYGVSPTKLNDLCWSSIYADYFVVKGDDVRQIAPAQTGVIADYNYKLPDDFTGCVLAWNTNSVDSKSGNNFYYRLFRHGKIKPYERDISNVLYNSAGGTCSSISQLGCYEPLKSYGFTPTVGVGYQPYRVVVLSFELLNAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 78 - protein, 477 aa, synthetic construct
MGILPSPGMPALLSLVSLLSVLLMGCVAETGTRFPNITNLCPFGQVFNASKFPSVYAWERLRISDCVADYSVLYNSSSSFSTFKCYGVSPTKLNDLCFSSVYADYFVVKGDDVRQIAPAQTGVIADYNYKLPDDFTGCVLAWNTNSVDSKQGNNFYYRLFRHGKIKPYERDISNVLYNSAGGTCSSTSQLGCYEPLKSYGFTPTVGVGYQPYRVVVLSFELLNAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 79 - protein, 474 aa, synthetic construct
MGILPSPGMPALLSLVSLLSVLLMGCVAETGTRFPNITQLCPFNEVFNITSFPSVYAWERMRITNCVADYSVLYNSSASFSTFQCYGVSPTKLNDLCFSSVYADYFVVKGDDVRQIAPAQTGVIADYNYKLPDDFTGCVIAWNTNSLDSSNEFFYRRFRHGKIKPYGRDLSNVLFNPSGGTCSAEGLNCYKPLASYGFTQSSGIGFQPYRVVVLSFELLNAPATVCGPKQSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 80 - protein, 459 aa, synthetic construct
MGILPSPGMPALLSLVSLLSVLLMGCVAETGTRFPNITNFCPFDKVFNATRFPNVYAWQRTKISDCIADYTVLYNSTSFSTFKCYGVSPSKLIDLCFTSVYADTFLIRFSEVRQIAPGETGVIADYNYKLPDDFTGCVLAWNTAQQDIGSYFYRSHRAVKLKPFERDLSSDENGVRTLSTYDFNPNVPLDYQATRVVVLSFELLNAPATVCGPKLSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 81 - protein, 442 aa, synthetic construct
MGILPSPGMPALLSLVSLLSVLLMGCVAETGTRFPNVYAWERTKISDCVADYTVLYNSTSFSTFKCYGVSPSKLIDLCFTSVYADTFLIRSSEVRQVAPGETGVIADYNYKLPDDFTGCVIAWNTAKHDTGNYYYRSHRKTKLKPFERDLSSDDGNGVYTLSTYDFNPNVPVAYQATRVVVLSFELLNAPATVCGPKLSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 82 - protein, 456 aa, synthetic construct
MGILPSPGMPALLSLVSLLSVLLMGCVAETGTRFPSVYAWTRERISNCIADYSVLYNSTSFSTFRCYGVSPTKLNDLCFSNVYADSMVVRGDEVRQIAPSQTGVIADYNYKLPDDFTGCVIAWNSKAKDENGQYFYRLFRKSKLLPFQRDVSNVTYGSGKNDGCNPSEADCYWPLLKYGFTSSVSQDYQPYRVVVLSFELLNAPATVCGPKRSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 83 - protein, 157 aa, synthetic construct
  VARIANT 1: note = optionally absent
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 84 - protein, 156 aa, synthetic construct
NQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAA

SEQ ID NO: 85 - protein, 157 aa, synthetic construct
  VARIANT 1: note = optionally absent
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVIDGMMNVQLDTGVPVLSAVLTPHRYRDSDAHTLLFLALFAVKGMEAARACVEILAAREKIAA

SEQ ID NO: 86 - protein, 157 aa, synthetic construct
  VARIANT 1: note = optionally absent
MNQHSHKDHETVRIAVVRARWHAEIVDACVSAFEAAMRDIGGDRFAVDVFDVPGAYEIPLHARTLAETGRYGAVLGTAFVVDGGIYDHEFVASAVIDGMMNVQLDTGVPVLSAVLTPHEYEDSDADTLLFLALFAVKGMEAARACVEILAAREKIAA

SEQ ID NO: 87 - protein, 32 aa, synthetic construct
MGILPSPGMPALLSLVSLLSVLLMGCVAETGT

SEQ ID NO: 88 - protein, 185 aa, synthetic construct
RFPNITNFCPFDKVFNATRFPNVYAWQRTKISDCIADYTVLYNSTSFSTFKCYGVSPSKLIDLCFTSVYADTFLIRFSEVRQIAPGETGVIADYNYKLPDDFTGCVLAWNTAQQDIGSYFYRSHRAVKLKPFERDLSSDENGVRTLSTYDFNPNVPLDYQATRVVVLSFELLNAPATVCGPKLST

SEQ ID NO: 89 - protein, 168 aa, synthetic construct
RFPNVYAWERTKISDCVADYTVLYNSTSFSTFKCYGVSPSKLIDLCFTSVYADTFLIRSSEVRQVAPGETGVIADYNYKLPDDFTGCVIAWNTAKHDTGNYYYRSHRKTKLKPFERDLSSDDGNGVYTLSTYDFNPNVPVAYQATRVVVLSFELLNAPATVCGPKLST

(19) United States
(12) Patent Application Publication
(10) Pub. No.: US 2026/0130986 A1
(43) Pub. Date: May 14, 2026

(54) PAN-SARBECOVIRUS NANOPARTICLE VACCINES

(71) Applicant: University of Washington, Seattle, WA (US)

(72) Inventors: Neil P. KING, Seattle, WA (US); David VEESLER, Seattle, WA (US); Alexandra C. WALLS, Seattle, WA (US); Marcos MIRANDA, Seattle, WA (US)

(21) Appl. No.: 19/118,077

(22) PCT Filed: Oct. 3, 2023
(86) PCT No.: PCT/US2023/075824

§ 371 Date: Apr. 3, 2025

Related U.S. Application Data
(60) Provisional application No. 63/378,410, filed on Oct. 5, 2022.

(51) Int. Cl. A61K 39/215; A61K 39/00; A61P 31/14; C12N 15/62

(52) U.S. Cl. CPC A61K 39/215; A61P 31/14; C12N 15/62; A61K 2039/55555; A61K 2039/70; C12N 2770/20034; C12N 2770/20071

(57) ABSTRACT

Manoparticies that display clade 1a, clade 1b, and clade 3 sarbecovirus receptor-binding domains and compositions thereof are provided, together with pharmaceutical compositions thereof and methods for using the nanoparticles and compositions to treat or limit Sarbecovirus infection.

### CROSS REFERENCE

This application claims priority to U.S. Provisional Patent Application Ser. No. 63/378,410 filed Oct. 5, 2022, incorporated by reference herein in its entirety.

### FEDERAL FUNDING STATEMENT

This invention was made with government support under Grant No. R01GM120553, awarded by the National Institutes of Health (NIH). The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Sep. 20, 2023 having the file name “22-1315-WO.xml” and is 14128.518 bytes in size

### BACKGROUND

The zoonotic emergence of severe acute respiratory syndrome coronavirus (SARS-CoV-1) in 2002 and SARS-CoV-2 in 2019 underscores that sarbecoviruses have and will continue to be a threat to global public health. SARS-CoV caused ˜8,000 infections worldwide and ˜800 deaths between 2002 and 2004. The emergence of SARS-CoV-2 in late 2019 resulted in the COVID-19 pandemic that has led to more than 500 million infected individuals and caused over 6 million deaths worldwide. The continued emergence of SARS-CoV-2 variants eroding infection- or vaccine-elicited antibody responses has thus far undermined efforts to end the pandemic. Given the high likelihood of future sarbecovirus spillover events and the limited protection against divergent sarbecoviruses or SARS-CoV-2 variants elicited by current SARS-CoV-2 vaccines, there is an urgent need for innovation and development of broad sarbecovirus countermeasures.

### SUMMARY

In one aspect, the disclosure comprises nanoparticles, wherein the nanoparticles displays on their surface an immunogenic portion of clade 1a, clade 1b, and clade 3 sarbecovirus receptor-binding domains (RBD), or variants thereof. In one embodiment, the nanoparticles comprise a plurality of fusion proteins, wherein the plurality of fusion proteins comprise a first domain comprising an amino acid sequence at least 80%, 85%, 90%, 91%, 92%, 93%, 94%, 95%, 96%, 97%, 98%, 99%, or 100% identical to the amino acid sequence of SEQ ID NO:1, fused to an RBD domain comprising:
(a) a clade 1a sarbecovirus RBD,
(ii) a clade 1b sarbecovirus RBD, and
(iii) a clade 3 sarbecovirus RBD;
wherein the N-terminal methionine residue of SEQ ID NO:1 is optional and may be present or absent.

In one embodiment, the RBD domains comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO:2-17. In another embodiment, the nanoparticles comprise a plurality of fusion proteins, wherein the plurality of fusion proteins comprise a first domain comprising an amino acid sequence at least 80%, 85%, 90%, 91%, 92%, 93%, 94%, 95%, 96%, 97%, 98%, 99%, or 100% identical to the amino acid sequence of SEQ ID NO:1, fused to:
(a) an amino acid at least 80%, 85%, 90%, 91%, 92%, 93%, 94%, 95%, 96%, 97%, 98%, 99%, or 100% identical to the amino acid sequence of SEQ ID NO:2 or 3 (clade 1b);
(b) an amino acid at least 80%, 85%, 90%, 91%, 92%, 93%, 94%, 95%, 96%, 97%, 98%, 99%, or 100% identical to the amino acid sequence of SEQ ID NO 13 (clade 1a); and
(c) an amino acid at least 80%, 85%, 90%, 91%, 92%, 93%, 94%, 95%, 96%, 97%, 98%, 99%, or 100% identical to the amino acid sequence of SEQ ID NO:16 or 17 (Clade 3);
wherein the N-terminal methionine residue of SEQ ID NO:1 is optional and may be present or absent.

In another embodiment, the nanoparticles display on their surface an immunogenic portion of at least a second clade 1b sarbecovirus RBD and/or a clade 2 sarbecovirus RBD, or variants thereof. In one embodiment, the plurality of fusion proteins comprises a first domain comprising an amino acid sequence at least 80%, 85%, 90%, 91%, 92%, 93%, 94%, 95%, 96%, 97%, 98%, 99%, or 100% identical to the amino acid sequence of SEQ ID NO:1, fused to an RBD domain comprising a second clade 1b sarbecovirus RBD or a clade 2 sarbecovirus RBD, wherein the N-terminal methionine residue of SEQ ID NO: 1 is optional and may be present or absent.

In another embodiment, the plurality of fusion proteins comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO:20-40, wherein optional linker residues may be present or absent, and if present may be substituted with any other linker. In a further embodiment, the plurality of fusion proteins comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO:41-61, wherein optional linker residues may be present or absent, and if present may be substituted with any other linker. In a still further embodiment, the plurality of fusion proteins comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO: 62-82.

In one embodiment, the nanoparticle comprises a plurality of fusion proteins, wherein the plurality of fusion proteins comprise a first domain comprising an amino acid sequence at least 95%, 96%, 97%, 98%, 99%, or 100% identical to the amino acid sequence of SEQ ID NO:1, fused to:
(a) an amino acid sequence at least 90%, 91%, 92%, 93%, 94%, 95%, 96%, 97%, 98%, 99%, or 100% identical to the amino acid sequence of SEQ ID NO:5 (clade 1b);
(b) an amino acid sequence at least 90%, 91%, 92%, 93%, 94%, 95%, 96%, 97%, 98%, 99%, or 100% identical to the amino acid sequence of SEQ ID NO:13 (clade 1a); and
(c) an amino acid sequence at least 90%, 91%, 92%, 93%, 94%, 95%, 96%, 97%, 98%, 99%, or 100% identical to the amino acid sequence of SEQ ID NO:16 or 17 (Clade 3);
wherein the N-terminal methionine residue of SEQ ID NO:1 is optional and may be present or absent. In another embodiment, the plurality of fusion proteins further comprise a first domain comprising an amino acid sequence at least 80%, 85%, 90%, 91%, 92%, 93%, 94%, 95%, 96%, 97%, 98%, 99%, or 100% identical to the amino acid sequence of SEQ ID NO:1, fused to:
(d) an amino acid sequence at least 80%, 85%, 90%, 91%, 92%, 93%, 94%, 95%, 96%, 97%, 98%, 99%, or 100% identical to the amino acid sequence of SEQ ID NO:2 (clade 1b);
wherein the N-terminal methionine residue of SEQ ID NO:1 is optional and may be present or absent.

In one embodiment, the nanoparticle comprises a plurality of fusion proteins, wherein the plurality of fusion proteins comprise a first domain comprising the amino acid sequence of SEQ ID NO:1, fused to:
(a) an amino acid sequence at least 80%, 85%, 90%, 91%, 92%, 93%, 94%, 95%, 96%, 97%, 98%, 99%, or 100% identical to the amino acid sequence of SEQ ID NO:5 (clade 1b);
(b) an amino acid sequence at least 80%, 85%, 90%, 91%, 92%, 93%, 94%, 95%, 96%, 97%, 98%, 99%, or 100% identical to the amino acid sequence of SEQ ID NO:13 (clade 1a);
(c) an amino acid sequence at least 80%, 85%, 90%, 91%, 92%, 93%, 94%, 95%, 96%, 97%, 98%, 99%, or 100% identical to the amino acid sequence of SEQ ID NO:16 or 17 (Clade 3); and
(d) an amino acid sequence at least 80%, 85%, 90%, 91%, 92%, 93%, 94%, 95%, 96%, 97%, 98%, 99%, or 100% identical to the amino acid sequence of SEQ ID NO:2 (clade 1b);
wherein the N-terminal methionine residue of SEQ ID NO:1 is optional and may be present or absent.

In another embodiment, the nanoparticles comprise
(a) a plurality of first assemblies, each first assembly comprising a plurality of identical first proteins comprising an amino acid sequence at least 80%, 85%, 90%, 91%, 92%, 93%, 94%, 95%, 96%, 97%, 98%, 99%, or 100% identical to the amino acid sequence of SEQ ID NO:83-86, or a plurality of first assemblies, each first assembly comprising a plurality of identical first proteins comprising an amino acid sequence at least 80%, 85%, 90%, 91%, 92%, 93%, 94%, 95%, 96%, 97%, 98%, 99%, or 100% identical to the amino acid sequence of SEQ ID NO: 83 or 84; and,
(b) a plurality of second assemblies, each second assembly comprising the plurality of fusion proteins of any embodiment or combination of embodiments herein;
wherein the plurality of first assemblies non-covalently interact with the plurality of second assemblies to form the nanoparticle; and
wherein the nanoparticle displays on its surface an immunogenic portion of clade 1a, clade 1b, and clade 3 sarbecovirus RBDs.

The disclosure also provides compositions comprising a plurality of nanoparticles of any embodiment herein.

In another aspect, the disclosure provides compositions comprising a plurality of nanoparticles, wherein the plurality of nanoparticles comprise:
(a) a first nanoparticle that comprises a fusion protein comprising a first domain comprising an amino acid sequence at least 95%, 96%, 97%, 98%, 99%, or 100% identical to the amino acid sequence of SEQ ID NO:1, fused to an amino acid sequence at least 90%, 91%, 92%, 93%, 94%, 95%, 96%, 97%, 98%, 99%, or 100% identical to the amino acid sequence of SEQ ID NO:5 (clade 1 b);
(b) a second nanoparticle that comprises a fusion protein comprising a first domain comprising an amino acid sequence at least 95%, 96%, 97%, 98%, 99%, or 100% identical to the amino acid sequence of SEQ ID NO:1, fused to an amino acid sequence at least 90%, 91%, 92%, 93%, 94%, 95%, 96%, 97%, 98%, 99%, or 100% identical to the amino acid sequence of SEQ ID NO: 13 (clade 1a); and
(c) a third nanoparticle that comprises a fusion protein comprising a first domain comprising an amino acid sequence at least 95%, 96%, 97%, 98%, 99%, or 100% identical to the amino acid sequence of SEQ ID NO:1, fused to an amino acid sequence at least 90%, 91%, 92%, 93%, 94%, 95%, 96%, 97%, 98%, 99%, or 100% identical to the amino acid sequence of SEQ ID NO:16 or 17 (Clade 3);
wherein the N-terminal methionine residue of SEQ ID NO:1 is optional and may be present or absent. In one embodiment, the composition further comprises a fourth nanoparticle that comprises a fusion protein comprising a first domain comprising an amino acid sequence at least 80%, 85%, 90%, 91%, 92%, 93%, 94%, 95%, 96%, 97%, 98%, 99%, or 100% identical to the amino acid sequence of SEQ ID NO:1, fused to an amino acid sequence at least 80%, 85%, 90%, 91%, 92%, 93%, 94%, 95%, 96%, 97%, 98%, 99%, or 100% identical to the amino acid sequence of SEQ ID NO:2 (clade 1b);
wherein the N-terminal methionine residue of SEQ ID NO:1 is optional and may be present or absent.

The disclosure also provides compositions comprising:
(a) a first nanoparticle that comprises a fusion protein comprising a first domain comprising an the amino acid sequence of SEQ ID NO:1, fused to an amino acid sequence at least 80%, 85%, 90%, 91%, 92%, 93%, 94%, 95%, 96%, 97%, 98%, 99%, or 100% identical to the amino acid sequence of SEQ ID NO:5 (clade 1b);
(b) a second nanoparticle that comprises a fusion protein comprising a first domain comprising the amino acid sequence of SEQ ID NO:1, fused to an amino acid sequence at least 80%, 85%, 90%, 91%, 92%, 93%, 94%, 950, 96%, 97%, 980, 99%, or 100% identical to the amino acid sequence of SEQ ID NO: 13 (clade 1a);
(c) q third nanoparticle that comprises a fusion protein comprising a first domain comprising the amino acid sequence of SEQ ID NO:1, fused to an amino acid sequence at least 80%, 85%, 90%, 91%, 92%, 93%, 94%, 95%, 96%, 97%, 98%, 99%, or 100% identical to the amino acid sequence of SEQ ID NO:16 or 17 (Clade 3); and
(d) a fourth nanoparticle that comprises a fusion protein comprising a first domain comprising the amino acid sequence of SEQ ID NO:1, fused to an amino acid sequence at least 80%, 85%, 90%, 91%, 92%, 93%, 94%, 95%, 96%, 97%, 98%, 99%, or 100% identical to the amino acid sequence of SEQ ID NO:2 (clade 1b)
wherein the N-terminal methionine residue of SEQ ID NO:1 is optional and may be present or absent.

The disclosure also provides nucleic acids encoding the fusion protein of any embodiment disclosed herein, or a plurality of nucleic acids molecules encoding the plurality of fusion proteins of any embodiment disclosed herein. In one embodiment, the nucleic acids comprise mRNAs. The disclosure further provides one or more expression vectors comprising the nucleic acid or the plurality of nucleic acid molecules of any embodiment disclosed herein operatively linked to a suitable control sequence. The disclosure also provides recombinant cells comprising the fusion proteins, nanoparticles, composition, nucleic acid, plurality of nucleic acid molecules, and/or one or more expression vector of any embodiment disclosed herein.

In a further embodiment, the disclosure provides pharmaceutical compositions or vaccines comprising
(a) the fusion proteins, nanoparticles, composition, plurality of nucleic acid molecules, one or more expression vector, and/or cell of any embodiment disclosed herein; and
(b) a pharmaceutically acceptable carrier.

The disclosure further provides methods to treat or limit development of a sarbecovirus infection, comprising administering to a subject in need thereof an amount effective to treat or limit development of the infection of the fusion proteins, nanoparticles, composition, plurality of nucleic acids, one or more expression vector, cell composition, vaccine, and or pharmaceutical composition of any embodiment disclosed herein.

In one embodiment, the disclosure provides kits, comprising
(a) the plurality of fusion proteins of any embodiment disclosed herein; and
(b) a protein comprising an amino acid sequence at least 80%, 85%, 90%, 91%, 92%, 93%, 94%, 95%, 96%, 97%, 98%, 99%, or 100% identical to the amino acid sequence of SEQ ID NO:83 or 84, wherein residues in parentheses are optional and may be present or absent;
(a1) a plurality of nucleic acid molecules encoding the fusion proteins of any embodiment disclosed herein operatively linked to a suitable control sequence; and
(b1) a nucleic acid encoding a protein comprising an amino acid sequence at least 80%, 85%, 90%, 91%, 92%, 93%, 94%, 95%, 96%, 97%, 98%, 99%, or 100% identical to the amino acid sequence of SEQ ID NO:83 or 84, wherein residues in parentheses are optional and may be present or absent, operatively linked to a suitable control sequence;
(a2) one or more expression vector comprising a plurality of nucleic acid molecules encoding the fusion proteins of any embodiment disclosed herein operatively linked to a suitable control sequence; and
(b2) an expression vector comprising a nucleic acid encoding a protein comprising an amino acid sequence at least 80%, 85%, 90%, 91%, 92%, 93%, 94%, 95%, 96%, 97%, 98%, 99%, or 100% identical to the amino acid sequence of SEQ ID NO:83 or 84, wherein residues in parentheses are optional and may be present or absent, operatively linked to a suitable control sequence; and/or
(a3) a cell comprising one or more expression vector, wherein the one or more expression vector comprises a plurality of nucleic acid molecules encoding the fusion proteins of any embodiment disclosed herein operatively linked to a suitable control sequence; and
(3b) a cell comprising an expression vector, wherein the expression vector a nucleic acid encoding a protein comprising an amino acid sequence at least 80%, 85%, 90%, 91%, 92%, 93%, 94%, 95%, 96%, 97%, 98%, 99%, or 100% identical to the amino acid sequence of SEQ ID NO:83 or 84, wherein residues in parentheses are optional and may be present or absent, operatively linked to a suitable control sequence.

### DESCRIPTION OF THE FIGURES

FIG. 1: Characterization of mosaic subunit vaccine candidates c/mRBD-Tri-NP and c/mRBD-Tetra-NP. (A) Sarbecovirus RBD amino acid sequence cladogram. RBDs present in the vaccine are SARS-CoV, SARS-CoV-2 (Wu-1), Omicron BA4/5, and BtKY72, and mismatched pseudoviruses used to evaluate the potency and breadth of vaccine-elicited antibodies are all others. (B) Schematic of in vitro assembly of mosaic (m) and cocktail (c) RBD-Tetra-NP and RBD-Tri-NP. RBD-Tri-NP contains RBDs from SARS-CoV, Omicron BA4/5, and BtKY72. RBD-Tetra-NP includes the same RBDs as RBD-Tri-NP in addition to SARS-CoV-2 Wu-1. (C) Evaluation of the hydrodynamic diameter of c/mRBD-Tetra-NP and c/mRBD-Tri-NP using dynamic light scattering. (D) Electron micrographs of negatively stained c/mRBD-Tetra-NP and c/mRBD-Tri-NP. Scale bar, 50 nim. (E) Biolayer interferometry. All receptors fused to human IgG Fc and monoclonal antibodies were immobilized on protein A biosensors and used to verify antigenicity of the immunogens. Human (h) ACE2 receptor and CR3022 mAb verified antigenicity of SARS-CoV, SARS-CoV-2 Wu-1, and Omicron BA4/5 RBDs. Mouse (m) ACE2 confirms antigenicity of Omicron BA4/5 RBD. LY-CoV555 mAb specifically binds SARS-CoV-2 Wu-1 RBD.

FIG. 2: Cocktail and mosaic RBD nanoparticle vaccines elicit broadly neutralizing sarbecovirus antibodies. (A & C) Representative study schematics of (B) & (D), respectively. (B & D) Serum neutralizing activity determined using a VSV pseudotyping system with VeroE6-TMPRSS2 cells (SARS-CoV-2 variants and SARS-CoV) or HEK293/ACE2 cells (BtKY72 DM, PRD-0038 DM, and Khosta1). VSV was pseudotyped with SARS-CoV-2 Wu-1 G614 S (Wu-1 G614), SARS-CoV-2 Omicron BA4/5 S, SARS-CoV-2 Omicron XBB1.5 S, SARS-CoV-2 Omicron BQ1.1 S, SARS-CoV S, BtKY72 DM S, PRD-0038 DM S, or Khosta1 S. Limit of detection (LOD).

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2nd Ed. (R. I. Freshney. 1987. Liss, Inc. New York, NY), Gene Transfer and Expression Protocols, pp. 109-128, ed. E. J. Murray, The Humana Press Inc., Clifton, N.J.), and the Ambion 1998 Catalog (Ambion, Austin, TX).

As used herein, the singular forms “a”, “an” and “the” include plural referents unless the context clearly dictates otherwise.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gin; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’. ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Any N-terminal methionine residues in polypeptides or polypeptide domains disclosed herein are optional and may be present or absent.

As used throughout the present application, the term “polypeptide” is used in its broadest sense to refer to a sequence of subunit D- or L-amino acids, including canonical and non-canonical amino acids. The polypeptides described herein may be chemically synthesized or recombinantly expressed. The polypeptides may be linked to other compounds to promote an increased half-life in vivo, such as by PEGylation, HESylation, PASylation, glycosylation, or may be produced as an Fc-fusion or in deimmunized variants. Such linkage can be covalent or non-covalent as is understood by those of skill in the art.

In a first aspect, the disclosure provides nanoparticles, wherein the nanoparticle displays on its surface an immunogenic portion of clade 1a, clade 1b, and clade 3 sarbecovirus receptor-binding domains (RBD), or variants thereof.

As is known by those of skill in the art, Sarbecovirus is a subgenus within the family Coronaviridae, and includes clades 1b, 1a, 3, and 2. Clade 1a and 1b sarbecoviruses caused two of the most recent spillovers of coronaviruses to humans, the SARS-CoV (Clade 1a) epidemic and SARS-CoV-2 (Clade 1b) pandemic. Some Clade 3 sarbecoviruses have recently been described to bind bat and human acetylcholinesterase 2 (ACE2) orthologs, similar to that of SARS-CoV and SARS-CoV-2. Clade 2 sarbecoviruses are mainly isolated from bat populations, which have been major reservoirs of zoonotic spillover. As discussed in the examples, recurrent zoonotic sarbecovirus spillovers along with the continued emergence of SARS-CoV-2 variants underscore the need to develop vaccines that elicit broad protection against these pathogens. Using a phylogeny-driven approach, the inventors designed mosaic nanoparticle receptor-binding domain vaccines that elicit potent and broad serum neutralizing antibody responses against vaccine-matched strains, including ancestral SARS-CoV-2, as well as genetically divergent sarbecoviruses that are not part of the vaccine formulation, including SARS-CoV-2 variants and other clade 1a, 1b, and 3 sarbecoviruses. As will be understood, the nanoparticle may further comprise RBDs (or variants thereof) from more than 1 clade 1a, clade 1b, and/or clade 3 sarbecovirus, and may comprise RBDs (or variants thereof) from other sarbecoviruses clades.

The nanoparticles can be used, for example, as mosaic vaccines to induce an immune response in individuals at risk of sarbecovirus infection.

The RBDs may be displayed on the surface of any nanoparticle appropriate for an intended use. In a specific embodiment, the nanoparticle comprises a plurality of fusion proteins, wherein the plurality of fusion proteins comprise a first domain comprising an amino acid sequence at least 80%, 85%, 90%, 91%, 92%, 93%, 94%, 95%, 96%, 97%, 98%, 99%, or 100% identical to the amino acid sequence of SEQ ID NO:1, fused to an RBD domain comprising:
(a) a clade 1a sarbecovirus RBD,
(ii) a clade 1b sarbecovirus RBD, and
(iii) a clade 3 sarbecovirus RBD;
wherein the N-terminal methionine residue of SEQ ID NO:1 is optional and may be present or absent.

(SEQ ID NO: 1; 153-50A)
(M)EELFKKHKIVAVLRANSVEEAIEKAVAVFAGGVHLIEITFTVPDADT
VIKALSVLKEKGAIIGAGTVTSVEQARKAVESGAEFIVSPHLDEEISQFA
KEKGVFYMPGVMTPTELVKAMKLGHTILKLFPGEVVGPQFVKAMKGPFPN
VKFVPTGGVNLDNVAEWFKAGVLAVGVGSALVKGTPDEVREKAKAFVEKI
RGATE

The polypeptide of SEQ ID NO:1 (also referred to as I53-50A), and variants thereof, can form two-component nanoparticles. In one embodiment, the nanoparticles comprise a
(a) a plurality of first assemblies, each first assembly comprising a plurality of identical first proteins comprising an amino acid sequence at least 80%, 85%, 90%, 91%, 92%, 93%, 94%, 95%, 96%, 97%, 98%, 99%, or 100% identical to the amino acid sequence of SEQ ID NO:83-86, or a plurality of first assemblies, each first assembly comprising a plurality of identical first proteins comprising an amino acid sequence at least 80%, 85%, 90%, 91%, 92%, 93%, 94%, 95%, 96%, 97%, 98%, 99%, or 100% identical to the amino acid sequence of SEQ ID NO: 83 or 84; and,
(b) a plurality of second assemblies, each second assembly comprising a plurality of identical fusion proteins according to any embodiment disclosed herein;
wherein the plurality of first assemblies non-covalently interact with the plurality of second assemblies to form the nanoparticle; and
wherein the nanoparticle displays on its surface an immunogenic portion of clade 1a, clade 1b, and clade 3 sarbecovirus RBDs.

The polypeptides of SEQ ID NO:83-86, and variants thereof, are known to form two-component nanoparticles when combined with as I53-50A or variants thereof. See, for example, U.S. Pat. No. 9,630,994 and WO/2019/094669, incorporated by reference herein in their entirety.

TABLE 1
(M)NQHSHKDHETVRIAVVRARWHAEIVDACVSAFEAAMRDIGGDRFAVD
VFDVPGAYEIPLHARTLAETGRYGAVLGTAFVVNGGIYRHEFVASAVING
MMNVQLNTGVPVLSAVLTPHNYDKSKAHTLLFLALFAVKGMEAARACVEI
LAAREKIAA (SEQ ID NO: 83); N-terminal Methionine
residue optional
NQHSHKDHETVRIAVVRARWHAEIVDACVSAFEAAMRDIGGDRFAVDVFD
VPGAYEIPLHARTLAETGRYGAVLGTAFVVNGGIYRHEFVASAVINGMMN
VQLNTGVPVLSAVLTPHNYDKSKAHTLLFLALFAVKGMEAARACVEILAA
REKIAA (SEQ ID NO: 84)
I53-50B.1
SEQ ID NO: 85
(M)NQHSHKDHETVRIAVVRARWHARIVDACVSAFEAAMRDIGGDRFAVD
VFDVPGAYEIPLHARTLAETGRYGAVLGTAFVVNGGIYRHEFVASAVIDG
MMNVQLDTGVPVLSAVLTPHRYRDSDAHTLLFLALFAVKGMEAARACVEI
LAAREKIAA N-terminal Methionine residue optional
I53-50B.1NegT2
SEQ ID NO: 86
(M)NQHSHKDHETVRIAVVRARWHAEIVDACVSAFEAAMRDIGGDRFAVD
VFDVPGAYEIPLHARTLAETGRYGAVLGTAFVVDGGIYDHEFVASAVIDG
MMNVQLDTGVPVLSAVLTPHEYEDSDADTLLFLALFAVKGMEAARACVEI
LAAREKIAA N-terminal Methionine residue optional

In this embodiment, the nanoparticle forms a three-dimensional structure formed by the non-covalent interaction of the first and second assemblies. A plurality (2, 3, 4, 5, 6, or more) of first polypeptides self-assemble to form a first assembly, and a plurality (2, 3, 4, 5, 6, or more) of second polypeptides self-assemble to form a second assembly. Non-covalent interaction of the individual self-assembling proteins results in self-assembly of the first protein into first assemblies, and self-assembly of the second proteins into second assemblies. A plurality of these first and second assemblies then self-assemble non-covalently via interfaces to produce the nanoparticles. The number of first polypeptides in the first assemblies may be the same or different than the number of second polypeptides in the second assemblies.

Assembly of the first and second assemblies into nanoparticles is not random, but is dictated by non-covalent interactions (e.g., hydrogen bonds, electrostatic, Van der Waals, hydrophobic, etc.) between the various assemblies (i.e., the cumulative effect of interactions between first assemblies, interactions between second assemblies, and interactions between first and second assemblies). Consequently, nanoparticles of this disclosure comprise symmetrically repeated, non-natural, non-covalent, protein-protein interfaces that orient the first and second assemblies into a nanoparticle having a highly ordered structure. While the formation of nanoparticles is due to non-covalent interactions of the first and second assemblies, in some embodiments, once formed, nanoparticles may be stabilized by covalent linking between proteins in the first assemblies and the second assemblies. Any suitable covalent linkage may be used, including but not limited to disulfide bonds and isopeptide linkages.

In one embodiment, the RBD domains comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO:2-19.

TABLE 2
Clade 1b
Wu1-RBD
RFPNITNLCPFGEVFNATRFASVYAWNRKRISNCVADYSVLYNSA
SFSTFKCYGVSPTKLNDLCFTNVYADSFVIRGDEVRQIAPGQTGK
IADYNYKLPDDFTGCVIAWNSNNLDSKVGGNYNYLYRLFRKSNLK
PFERDISTEIYQAGSTPCNGVEGFNCYFPLQSYGFQPTNGVGYQP
YRVVVLSFELLHAPATVCGPKKST (SEQ ID NO: 2)
Wu1Rpk9-RBD
RFPNITNLCPFGEVFNATRFASVYAWNRKRISNCVADFSVLYNSA
SFSTFKCYGVSPTKLNDLCWTNIYADSFVIRGDEVRQIAPGQTGK
IADYNYKLPDDFTGCVIAWNSNNLDSKVGGNYNYLYRLFRKSNLK
PFERDISTEIYQAGSTPCNGVEGFNCYFPLQSYGFQPTNGVGYQP
YRVVVLSFELLHAPATVCGPKKST (SEQ ID NO: 3)
OmicronBA.4/5-RBD
RFPNITNLCPFDEVFNATRFASVYAWNRKRISNCVADYSVLYNFA
PFFAFKCYGVSPTKLNDLCFTNVYADSFVIRGNEVSQIAPGQTGN
IADYNYKLPDDFTGCVIAWNSNKLDSKVGGNYNYRYRLFRKSNLK
PFERDISTEIYQAGNKPCNGVAGVNCYFPLQSYGFRPTYGVGHQP
YRVVVLSFELLHAPATVCGPKKST (SEQ ID NO: 4)
OmicronBA.4/5Rpk9-RBD
RFPNITNLCPFDEVFNATRFASVYAWNRKRISNCVADFSVLYNFA
PFFAFKCYGVSPTKLNDLCWTNIYADSFVIRGNEVSQIAPGQTGN
IADYNYKLPDDFTGCVIAWNSNKLDSKVGGNYNYRYRLFRKSNLK
PFERDISTEIYQAGNKPCNGVAGVNCYFPLQSYGFRPTYGVGHQP
YRVVVLSFELLHAPATVCGPKKST (SEQ ID NO: 5)
OmicronBA.1-RBD
RFPNITNLCPFDEVFNATRFASVYAWNRKRISNCVADYSVLYNLA
PFFTFKCYGVSPTKLNDLCFINVYADSFVIRGDEVRQIAPGQTGN
IADYNYKLPDDFTGCVIAWNSNKLDSKVSGNYNYLYRLFRKSNLK
PFERDISTEIYQAGNKPCNGVAGFNCYFPLRSYSFRPTYGVGHQP
YRVVVLSFELLHAPATVCGPKKST (SEQ ID NO: 6)
OmicronBA.1Rpk9-RBD
RFPNITNLCPFDEVFNATRFASVYAWNRKRISNCVADFSVLYNLA
PFFTFKCYGVSPTKLNDLCWTNIYADSFVIRGDEVRQIAPGQTGN
IADYNYKLPDDFTGCVIAWNSNKLDSKVSGNYNYLYRLFRKSNLK
PFERDISTEIYQAGNKPCNGVAGFNCYFPLRSYSFRPTYGVGHQP
YRVVVLSFELLHAPATVCGPKKST (SEQ ID NO: 7)
OmicronBA.2-RBD
RFPNITNLCPFDEVFNATRFASVYAWNRKRISNCVADYSVLYNFA
PFFAFKCYGVSPTKLNDLCFTNVYADSFVIRGNEVSQIAPGQTGN
IADYNYKLPDDFTGCVIAWNSNKLDSKVGGNYNYLYRLFRKSNLK
PFERDISTEIYQAGNKPCNGVAGFNCYFPLRSYGFRPTYGVGHQP
YRVVVLSFELLHAPATVCGPKKST (SEQ ID NO: 8)
OmicronBA.2Rpk9-RBD
RFPNITNLCPFDEVFNATRFASVYAWNRKRISNCVADFSVLYNFA
PFFAFKCYGVSPTKLNDLCWTNIYADSFVIRGNEVSQIAPGQTGN
IADYNYKLPDDFTGCVIAWNSNKLDSKVGGNYNYLYRLFRKSNLK
PFERDISTEIYQAGNKPCNGVAGFNCYFPLRSYGFRPTYGVGHQP
YRVVVLSFELLHAPATVCGPKKST (SEQ ID NO: 9)
Delta-RBD
REPNITNLCPFGEVFNATRFASVYAWNRKRISNCVADYSVLYNSA
SFSTFKCYGVSPTKLNDLCFTNVYADSFVIRGDEVRQIAPGQTGK
IADYNYKLPDDFTGCVIAWNSNNLDSKVGGNYNYRYRLFRKSNLK
PFERDISTEIYQAGSKPCNGVEGFNCYFPLQSYGFQPINGVGYQP
YRVVVLSFELLHAPATVCGPKKST (SEQ ID NO: 10)
RaTG13-RBD
RFPNITNLCPFGEVFNATTFASVYAWNRKRISNCVADYSVLYNST
SFSTEKCYGVSPTKLNDLCFTNVYADSFVITGDEVRQIAPGQTGK
IADYNYKLPDDFTGCVIAWNSKHIDAKEGGNFNYLYRLFRKANLK
PFERDISTEIYQAGSKPCNGQTGLNCYYPLYRYGFYPTDGVGHQP
YRVVVLSFELLNAPATVCGPKKST (SEQ ID NO: 11)
PangolinGX-RBD
RFPNITNLCPFGEVFNASKFASVYAWNRKRISNCVADYSVLYNST
SFSTFKCYGVSPTKLNDLCFTNVYADSFVVKGDEVRQIAPGQTGV
IADYNYKLPDDFTGCVIAWNSVKQDALTGGNYLYRLFRKSKLKPF
ERDISTEIYQAGSTPCNGQVGLNCYYPLERYGFHPTTGVNYQPFR
VVVLSFELLNGPATVCGPKLST (SEQ ID NO: 12)
Clade 1a
SARS1-RBD
RFPNITNLCPFGEVFNATKFPSVYAWERKKISNCVADYSVLYNST
FFSTFKCYGVSATKLNDLCFSNVYADSFVVKGDDVRQIAPGQTGV
IADYNYKLPDDFMGCVLAWNTRNIDATSTGNYNYKYRYLRHGKLR
PFERDISNVPFSPDGKPCTPPALNCYWPLNDYGFYTTTGIGYQPY
RVVVLSFELLNAPATVCGPKLST (SEQ ID NO: 13)
SHC014-RBD
RFPNITNLCPFGEVFNATTFPSVYAWERKRISNCVADYSVLYNST
SFSTFKCYGVSATKLNDLCFSNVYADSFVVKGDDVRQIAPGQTGV
IADYNYKLPDDFLGCVLAWNTNSKDSSTSGNYNYLYRWVRRSKIN
PYERDLSNDIYSPGGQSCSAVGPNCYNPLRPYGFFTTAGVGHQPY
RVVVLSFELLNAPATVCGPKLST (SEQ ID NO: 14)
WIV1/16-RBD
RFPNITNLCPFGEVFNATTEPSVYAWERKRISNCVADYSVLYNST
SFSTFKCYGVSATKINDLCESNVYADSFVVKGDDVRQIAPGQTGV
IADYNYKLPDDFTGCVLAWNTRNIDATQTGNYNYKYRSLRHGKLR
PFERDISNVPFSPDGKPCTPPAFNCYWPLNDYGFYITNGIGYQPY
RVVVLSFELLNAPATVCGPKLST (SEQ ID NO: 15)
Clade 3
BtKY72-RBD
RFPNITNLCPFGQVFNASNFPSVYAWERLRISDCVADYAVLYNSS
SSFSTFKCYGVSPTKLNDLCFSSVYADYFVVKGDDVRQIAPAQTG
VIADYNYKLPDDFTGCVLAWNTNSVDSKSGNNFYYRLFRAGKIKP
YERDISNVLYNSAGGTCSSISQLGCYEPLKSYGFTPTVGVGYQPY
RVVVLSFELLNAPATVCGPKKST (SEQ ID NO: 16)
BtKY72Rpk9-RBD
RFPNITNLCPFGQVFNASNFPSVYAWERLRISDCVADFAVLYNSS
SSFSTFKCYGVSPTKLNDLCWSSIYADYFVVKGDDVRQIAPAQTG
VIADYNYKLPDDFTGCVLAWNTNSVDSKSGNNFYYRLFRHGKIKP
YERDISNVLYNSAGGTCSSISQLGCYEPLKSYGFTPTVGVGYQPY
RVVVLSFELLNAPATVCGPKKST (SEQ ID NO: 17)
PRD0038-RBD
RFPNITNLCPFGQVFNASKFPSVYAWERLRISDCVADYSVLYNSS
SSFSTFKCYGVSPTKLNDLCFSSVYADYFVVKGDDVRQIAPAQTG
VIADYNYKLPDDFTGCVLAWNTNSVDSKQGNNFYYRLFRHGKIKP
YERDISNVLYNSAGGTCSSTSQLGCYEPLKSYGFTPTVGVGYQPY
RVVVLSFELLNAPATVCGPKKST (SEQ ID NO: 18)
BM48-31-RBD
RFPNITQLCPFNEVFNITSFPSVYAWERMRITNCVADYSVLYNSS
ASFSTFQCYGVSPTKLNDLCFSSVYADYFVVKGDDVRQIAPAQTG
VIADYNYKLPDDFTGCVIAWNTNSLDSSNEFFYRRFRHGKIKPYG
RDLSNVLFNPSGGTCSAEGLNCYKPLASYGFTQSSGIGFQPYRVV
VLSFELLNAPATVCGPKQST (SEQ ID NO: 19)

In another embodiment, the nanoparticle comprises a plurality of fusion proteins, wherein the plurality of fusion proteins comprise a first domain comprising an amino acid sequence at least 80%, 85%, 90%, 91%, 92%, 93%, 94%, 95%, 96%, 97%, 98%, 99%, or 100% identical to the amino acid sequence of SEQ ID NO:1, fused to:
(a) an amino acid at least 80%, 85%, 90%, 91%, 92%, 93%, 94%, 95%, 96%, 97%, 98%, 99%, or 100% identical to the amino acid sequence of SEQ ID NO:5 (clade 1b);
(b) an amino acid at least 80%, 85%, 90%, 91%, 92%, 93%, 94%, 95%, 96%, 97%, 98%, 99%, or 100% identical to the amino acid sequence of SEQ ID NO:13 (clade 1a); and
(c) an amino acid at least 80%, 85%, 90%, 91%, 92%, 93%, 94%, 95%, 96%, 97%, 98%, 99%, or 100% identical to the amino acid sequence of SEQ ID NO:16 or 17 (Clade 3);
wherein the N-terminal methionine residue of SEQ ID NO:1 is optional and may be present or absent.

In one embodiment, the nanoparticle comprises a plurality of fusion proteins, wherein the plurality of fusion proteins comprise a first domain comprising an amino acid sequence at least 80%, 85%, 90%, 91%, 92%, 93%, 94%, 95%, 96%, 97%, 98%, 99%, or 100% identical to the amino acid sequence of SEQ ID NO:1, fused to:
(a) an amino acid at least 80%, 85%, 90%, 91%, 92%, 93%, 94%, 95%, 96%, 97%, 98%, 99%, or 100% identical to the amino acid sequence of SEQ ID NO:2 or 3 (clade 1b);
(b) an amino acid at least 80%, 85%, 90%, 91%, 92%, 93%, 94%, 95%, 96%, 97%, 98%, 99%, or 100% identical to the amino acid sequence of SEQ ID NO 13 (clade 1a); and
(c) an amino acid at least 80%, 85%, 90%, 91%, 92%, 93%, 94%, 95%, 96%, 97%, 98%, 99%, or 100% identical to the amino acid sequence of SEQ ID NO:16 or 17 (Clade 3);
wherein the N-terminal methionine residue of SEQ ID NO:1 is optional and may be present or absent.

The nanoparticles may display additional sarbecovirus RBDs or immunogenic portions thereof. In one embodiment, the nanoparticle displays on its surface an immunogenic portion of at least a second clade 1b sarbecovirus RBD and/or a clade 2 sarbecovirus RBD, or variants thereof. In another embodiment, the plurality of fusion proteins comprises a first domain comprising an amino acid sequence at least 80%, 85%, 90%, 91%, 92%, 93%, 94%, 95%, 96%, 97%, 98%, 99%, or 100% identical to the amino acid sequence of SEQ ID NO:1, fused to an RBD domain comprising a second clade 1b sarbecovirus RBD or a clade 2 sarbecovirus RBD, wherein the N-terminal methionine residue of SEQ ID NO:1 is optional and may be present or absent. In other embodiments, a second clade 1b RBD, or the clade 2 RBD comprises an amino acid at least 80%, 85%, 90%, 91%, 92%, 93%, 94%, 95%, 96%, 97%, 98%, 99%, or 100% identical to the amino acid sequence selected from the group consisting of SEQ ID NO:4-9 and 88-89.

TABLE 3

| | |
|---|---|
| RmYN02- | RFPNITNFCPFDKVFNATRFPNVYAWQRTKISD |
| RBD-I53- | CIADYTVLYNSTSFSTFKCYGVSPSKLIDLCFT |
| 50A | SVYADTFLIRFSEVRQIAPGETGVIADYNYKLP |
| SEQ ID: | DDFTGCVLAWNTAQQDIGSYFYRSHRAVKLKPF |
| 88 | ERDLSSDENGVRTLSTYDFNPNVPLDYQATRVV |
| | VLSFELLNAPATVCGPKLST |
| HKU3-RBD- | RFPNVYAWERTKISDCVADYTVLYNSTSFSTFK |
| I53-50A | CYGVSPSKLIDLCFTSVYADTFLIRSSEVRQVA |
| SEQ ID: | PGETGVIADYNYKLPDDFTGCVIAWNTAKHDTG |
| 89 | NYYYRSHRKTKLKPFERDLSSDDGNGVYTLSTY |
| | DFNPNVPVAYQATRVVVLSFELLNAPATVCGPK |
| | LST |

In another embodiment, a second clade 1b sarbecovirus RBD or the clade 2 sarbecovirus RBD comprises an amino acid at least 80%, 85%, 90%, 91%, 92%, 93%, 94%, 95%, 96%, 97%, 98%, 99%, or 100% identical to the amino acid sequence of SEQ ID NO:4-5 and 88.

The fusion proteins of any aspect or embodiment disclosed herein may comprises an amino acid linker between the first domain and the RBD domain. As used throughout this disclosure, a linker is a short (e.g., 2-30) amino acid sequence used to covalently join two polypeptides. Any suitable linker sequence may be used, including but not limited to those disclosed herein.

In a further embodiment, the plurality of fusion proteins comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO:20-40, wherein underlined residues are linkers and may be present or absent, and if present may be substituted with any other linker.

TABLE 4

| | |
|---|---|
| Wu1-RBD- | RFPNITNLCPFGEVFNATRFASVYAWNRKRISNCVADYSVLYNSASFSTFKCYGVSPTKLN |
| I53-50A | DLCFTNVYADSFVIRGDEVRQIAPGQTGKIADYNYKLPDDFTGCVIAWNSNNLDSKVGGNY |
| SEQ ID | NYLYRLFRKSNLKPFERDISTEIYQAGSTPCNGVEGFNCYFPLQSYGFQPTNGVGYQPYRV |
| NO: 20 | VVLSFELLHAPATVCGPKKSTGGSGGSGSGGSGGSGSEKAAKAEEAARKMEELFKKHKIVA |
| | VLRANSVEEAIEKAVAVFAGGVHLIEITFTVPDADTVIKALSVLKEKGAIIGAGTVTSVEQ |
| | ARKAVESGAEFIVSPHLDEEISQFAKEKGVFYMPGVMTPTELVKAMKLGHTILKLFPGEVV |
| | GPQFVKAMKGPFPNVKFVPTGGVNLDNVAEWFKAGVLAVGVGSALVKGTPDEVREKAKAFV |
| | EKIRGATEc |
| Wu1Rpk9- | RFPNITNLCPFGEVFNATRFASVYAWNRKRISNCVADFSVLYNSASFSTFKCYGVSPTKLN |
| RBD-I53- | DLCWTNIYADSFVIRGDEVRQIAPGQTGKIADYNYKLPDDFTGCVIAWNSNNLDSKVGGNY |
| 50A | NYLYRLFRKSNLKPFERDISTEIYQAGSTPCNGVEGFNCYFPLQSYGFQPTNGVGYQPYRV |
| SEQ ID | VVLSFELLHAPATVCGPKKSTGGSGGSGSGGSGGSGSEKAAKAEEAARKMEELFKKHKIVA |
| NO: 21 | VLRANSVEEAIEKAVAVFAGGVHLIEITFTVPDADTVIKALSVLKEKGAIIGAGTVTSVEQ |
| | ARKAVESGAEFIVSPHLDEEISQFAKEKGVFYMPGVMTPTELVKAMKLGHTILKLFPGEVV |
| | GPQFVKAMKGPFPNVKFVPTGGVNLDNVAEWEKAGVLAVGVGSALVKGTPDEVREKAKAFV |
| | EKIRGATE |
| OmicronB | RFPNITNLCPFDEVFNATRFASVYAWNRKRISNCVADYSVLYNFAPFFAFKCYGVSPTKLN |
| A.4/5- | DLCFTNVYADSFVIRGNEVSQIAPGQTGNIADYNYKLPDDFTGCVIAWNSNKLDSKVGGNY |
| RBD-I53- | NYRYRLFRKSNLKPFERDISTEIYQAGNKPCNGVAGVNCYFPLQSYGFRPTYGVGHQPYRV |
| 50A | VVLSFELLHAPATVCGPKKSTGGSGGSGSGGSGGSGSEKAAKAEEAARKMEELFKKHKIVA |
| SEQ ID | VLRANSVEEAIEKAVAVFAGGVHLIEITFTVPDADTVIKALSVLKEKGAIIGAGTVTSVEQ |
| NO: 22 | ARKAVESGAEFIVSPHLDEEISQFAKEKGVFYMPGVMTPTELVKAMKLGHTILKLFPGEVV |
| | GPQFVKAMKGPFPNVKFVPTGGVNLDNVAEWFKAGVLAVGVGSALVKGTPDEVREKAKAFV |
| | EKIRGATE |
| OmicronB | RFPNITNLCPFDEVFNATRFASVYAWNRKRISNCVADFSVLYNFAPFFAFKCYGVSPTKLN |
| A.4/5Rpk | DLCWTNIYADSFVIRGNEVSQIAPGQTGNIADYNYKLPDDFTGCVIAWNSNKLDSKVGGNY |
| 9-RBD- | NYRYRLFRKSNLKPFERDISTEIYQAGNKPCNGVAGVNCYFPLQSYGFRPTYGVGHQPYRV |
| I53-50A | VVLSFELLHAPATVCGPKKSTGGSGGSGSGGSGGSGSEKAAKAEEAARKMEELFKKHKIVA |
| SEQ ID | VLRANSVEEAIEKAVAVFAGGVHLIEITFTVPDADTVIKALSVLKEKGAIIGAGTVTSVEQ |
| NO: 23 | ARKAVESGAEFIVSPHLDEEISQFAKEKGVFYMPGVMTPTELVKAMKLGHTILKLFPGEVV |
| | GPQFVKAMKGPFPNVKFVPTGGVNLDNVAEWFKAGVLAVGVGSALVKGTPDEVREKAKAFV |
| | EKIRGATE |
| OmicronB | RFPNITNLCPFDEVFNATRFASVYAWNRKRISNCVADYSVLYNLAPFFTFKCYGVSPTKLN |
| A.1-RBD- | DLCFTNVYADSFVIRGDEVRQIAPGQTGNIADYNYKLPDDFTGCVIAWNSNKLDSKVSGNY |
| I53-50A | NYLYRLFRKSNLKPFERDISTEIYQAGNKPCNGVAGFNCYFPLRSYSFRPTYGVGHQPYRV |
| SEQ ID | VVLSFELLHAPATVCGPKKSTGGSGGSGSGGSGGSGSEKAAKAEEAARKMEELFKKAKIVA |
| NO: 24 | VLRANSVEEAIEKAVAVFAGGVHLIEITFTVPDADTVIKALSVLKEKGAIIGAGTVTSVEQ |
| | ARKAVESGAEFIVSPHLDEEISQFAKEKGVFYMPGVMTPTELVKAMKLGHTILKLFPGEVV |
| | GPQFVKAMKGPFPNVKFVPTGGVNLDNVAEWFKAGVLAVGVGSALVKGTPDEVREKAKAFV |
| | EKIRGATE |
| OmicronB | RFPNITNLCPFDEVFNATRFASVYAWNRKRISNCVADFSVLYNLAPFFTFKCYGVSPTKLN |
| A.1Rpk9- | DLCWTNIYADSFVIRGDEVRQIAPGQTGNIADYNYKLPDDFTGCVIAWNSNKLDSKVSGNY |
| RBD-I53- | NYLYRLFRKSNLKPFERDISTEIYQAGNKPCNGVAGFNCYFPLRSYSFRPTYGVGHQPYRV |
| 50A | VVLSFELLHAPATVCGPKKSTGGSGGSGSGGSGGSGSEKAAKAREAARKMEELFKKHKIVA |
| SEQ ID | VLRANSVEEAIEKAVAVFAGGVHLIEITFTVPDADTVIKALSVLKEKGAIIGAGTVTSVEQ |
| NO: 25 | ARKAVESGAEFIVSPHLDEEISQFAKEKGVFYMPGVMTPTELVKAMKLGHTILKLFPGEVV |
| | GPQFVKAMKGPFPNVKFVPTGGVNLDNVAEWFKAGVLAVGVGSALVKGTPDEVREKAKAFV |
| | EKIRGATE |
| OmicronB | RFPNITNLCPFDEVFNATRFASVYAWNRKRISNCVADYSVLYNFAPFFAFKCYGVSPTKLN |
| A.2-RBD- | DLCFTNVYADSFVIRGNEVSQIAPGQTGNIADYNYKLPDDFTGCVIAWNSNKLDSKVGGNY |
| I53-50A | NYLYRLFRKSNLKPFERDISTEIYQAGNKPCNGVAGFNCYFPLRSYGFRPTYGVGHQPYRV |
| SEQ ID | VVLSFELLHAPATVCGPKKSTGGSGGSGSGGSGGSGSEKAAKAEEAARKMEELFKKHKIVA |
| NO: 26 | VLRANSVEEAIEKAVAVFAGGVHLIEITFTVPDADTVIKALSVLKEKGAIIGAGTVTSVEQ |
| | ARKAVESGAEFIVSPHLDEEISQFAKEKGVFYMPGVMTPTELVKAMKLGHTILKLFPGEVV |
| | GPQFVKAMKGPFPNVKFVPTGGVNLDNVAEWFKAGVLAVGVGSALVKGTPDEVREKAKAFV |
| | EKIRGATE |
| OmicronB | RFPNITNLCPFDEVFNATREASVYAWNRKRISNCVADFSVLYNFAPFFAFKCYGVSPTKLN |
| A.Rpk9- | DLCWTNIYADSFVIRGNEVSQIAPGQTGNIADYNYKLPDDFTGCVIAWNSNKLDSKVGGNY |
| RBD-I53- | NYLYRLERKSNLKPFERDISTEIYQAGNKPCNGVAGFNCYFPLRSYGFRPTYGVGHQPYRV |
| 50A | VVLSFELLHAPATVCGPKKSTGGSGGSGSGGSGGSGSEKAAKAEEAARKMEELFKKHKIVA |
| SEQ ID | VLRANSVEEAIEKAVAVFAGGVHLIEITFTVPDADTVIKALSVLKEKGAIIGAGTVTSVEQ |
| NO: 27 | ARKAVESGAEFIVSPHLDEEISQFAKEKGVFYMPGVMTPTELVKAMKLGHTILKLFPGEVV |
| | GPQFVKAMKGPFPNVKFVPTGGVNLDNVAEWFKAGVLAVGVGSALVKGTPDEVREKAKAFV |
| | EKIRGATE |
| Delta- | RFPNITNLCPFGEVFNATRFASVYAWNRKRISNCVADYSVLYNSASFSTFKCYGVSPTKLN |
| RBD-I53- | DLCFTNVYADSFVIRGDEVRQIAPGQTGKIADYNYKLPDDFTGCVIAWNSNNLDSKVGGNY |
| 50A | NYRYRLFRKSNLKPFERDISTEIYQAGSKPCNGVEGFNCYFPLQSYGFQPTNGVGYQPYRV |
| SEQ ID | VVLSFELLHAPATVCGPKKSTGGSGGSGSGGSGGSGSEKAAKAEEAARKMEELFKKHKIVA |
| NO: 28 | VLRANSVEEAIEKAVAVFAGGVHLIEITFTVPDADTVIKALSVLKEKGAIIGAGTVTSVEQ |
| | ARKAVESGAEFIVSPHLDEEISQFAKEKGVFYMPGVMTPTELVKAMKLGHTILKLFPGEVV |
| | GPQFVKAMKGPFPNVKFVPTGGVNLDNVAEWFKAGVLAVGVGSALVKGTPDEVREKAKAFV |
| | EKIRGATE |
| RaTG13- | RFPNITNLCPFGEVFNATTFASVYAWNRKRISNCVADYSVLYNSTSFSTFKCYGVSPTKLN |
| RED-I53- | DLCFTNVYADSFVITGDEVRQIAPGQTGKIADYNYKLPDDFTGCVIAWNSKHIDAKEGGNF |
| 50A | NYLYRLFRKANLKPFERDISTEIYQAGSKPCNGQTGLNCYYPLYRYGFYPTDGVGHQPYRV |
| SEQ ID | VVLSFELLNAPATVCGPKKSTGGSGGSGSGGSGGSGSEKAAKAEEAARKMEELFKKHKIVA |
| NO: 29 | VLRANSVEEATEKAVAVFAGGVHLIEITFTVPDADTVIKALSVLKEKGAIIGAGTVTSVEQ |
| | ARKAVESGAEFIVSPHLDEEISQFAKEKGVFYMPGVMTPTELVKAMKLGHTILKLFPGEVV |
| | GPQFVKAMKGPFPNVKFVPTGGVNLDNVAEWFKAGVLAVGVGSALVKGTPDEVREKAKAFV |
| | EKIRGATE |
| Pangolin | RFPNITNLCPFGEVFNASKFASVYAWNRKRISNCVADYSVLYNSTSFSTFKCYGVSPTKLN |
| GX-RBD- | DLCFTNVYADSFVVKGDEVRQIAPGQTGVIADYNYKLPDDFTGCVIAWNSVKQDALTGGNY |
| I53-50A | LYRLFRKSKLKPFERDISTEIYQAGSTPCNGQVGLNCYYPLERYGFHPTTGVNYQPFRVVV |
| SEQ ID | LSFELLNGPATVCGPKLSTGGSGGSGSGGSGGSGSEKAAKAEEAARKMEELFKKHKIVAVL |
| NO: 30 | RANSVEEAIEKAVAVFAGGVHLIEITFTVPDADTVIKALSVLKEKGAIIGAGTVTSVEQAR |
| | KAVESGAEFIVSPHLDEEISQFAKEKGVFYMPGVMTPTELVKAMKLGHTILKLFPGEVVGP |
| | QFVKAMKGPFPNVKFVPTGGVNLDNVAEWFKAGVLAVGVGSALVKGTPDEVREKAKAFVEK |
| | IRGATE |
| SARS1- | RFPNITNLCPFGEVFNATKFPSVYAWERKKISNCVADYSVLYNSTFFSTEKCYGVSATKLN |
| RBD-I53- | DLCFSNVYADSFVVKGDDVRQIAPGQTGVIADYNYKLPDDFMGCVLAWNTRNIDATSTGNY |
| 50A | NYKYRYLRHGKLRPFERDISNVPFSPDGKPCTPPALNCYWPINDYGFYTTTGIGYQPYRVV |
| SEQ ID | VLSFELLNAPATVCGPKLSTGGSGGSGSGGSGGSGSEKAAKAEEAARKMEELFKKHKIVAV |
| NO: 31 | LRANSVEEAIEKAVAVFAGGVHLIEITFTVPDADTVIKALSVLKEKGAIIGAGTVTSVEQA |
| | RKAVESGAEFIVSPHLDEEISQFAKEKGVFYMPGVMTPTELVKAMKLGHTILKLEPGEVVG |
| | PQFVKAMKGPFPNVKFVPTGGVNLDNVAEWFKAGVLAVGVGSALVKGTPDEVREKAKAFVE |
| | KIRGATE |
| SHC014- | RFPNITNLCPFGEVFNATTFPSVYAWERKRISNCVADYSVLYNSTSFSTFKCYGVSATKLN |
| RBD-I53- | DLCFSNVYADSFVVKGDDVRQIAPGQTGVIADYNYKLPDDFLGCVLAWNTNSKDSSTSGNY |
| 50A | NYLYRWVRRSKLNPYERDLSNDIYSPGGQSCSAVGPNCYNPLRPYGFFTTAGVGHQPYRVV |
| SEQ ID | VLSFELLNAPATVCGPKLSTGGSGGSGSGGSGGSGSEKAAKAEEAARKMEELFKKHKIVAV |
| NO: 32 | LRANSVEEAIEKAVAVFAGGVHLIEITFTVPDADTVIKALSVLKEKGAIIGAGTVTSVEQA |
| | RKAVESGAEFIVSPHLDEEISQFAKEKGVFYMPGVMTPTELVKAMKLGHTILKLEPGEVVG |
| | PQFVKAMKGPFPNVKFVPTGGVNLDNVAEWFKAGVLAVGVGSALVKGTPDEVREKAKAFVE |
| | KIRGATE |
| WIV1/16- | RFPNITNLCPFGEVFNATTFPSVYAWERKRISNCVADYSVLYNSTSFSTFKCYGVSATKLN |
| RBD-I53- | DICFSNVYADSFVVKGDDVRQIAPGQTGVIADYNYKLPDDETGCVLAWNTRNIDATQTGNY |
| 50.A | NYKYRSLRHGKLRPFERDISNVPFSPDGKPCTPPAFNCYWPLNDYGFYITNGIGYQPYRVV |
| SEQ ID | VLSFELLNAPATVCGPKLSTGGSGGSGSGGSGGSGSEKAAKAEEAARKMEELFKKHKIVAV |
| NO: 33 | LRANSVEEAIEKAVAVFAGGVHLIEITFTVPDADTVIKALSVLKEKGAIIGAGTVTSVEQA |
| | RKAVESGAEFIVSPHLDEEISQFAKEKGVFYMPGVMTPTELVKAMKLGHTILKLFPGEVVG |
| | PQFVKAMKGPFPNVKFVPTGGVNLDNVAEWFKAGVLAVGVGSALVKGTPDEVREKAKAFVE |
| | KIRGATE |
| BtKY72- | RFPNITNLCPFGQVFNASNFPSVYAWERLRISDCVADYAVLYNSSSSFSTFKCYGVSPTKL |
| RBD-I53- | NDLCFSSVYADYFVVKGDDVRQIAPAQTGVIADYNYKLPDDFTGCVLAWNTNSVDSKSGNN |
| 50A | FYYRLFRHGKIKPYERDISNVLYNSAGGTCSSISQLGCYEPLKSYGFTPTVGVGYQPYRVV |
| SEQ ID | VLSFELLNAPATVCGPKKSTGGSGGSGSGGSGGSGSEKAAKAEEAARKMEELFKKHKIVAV |
| NO: 34 | LRANSVEEAIEKAVAVFAGGVHLIEITFTVPDADTVIKALSVLKEKGAIIGAGTVTSVEQA |
| | RKAVESGAEFIVSPHLDEEISQFAKEKGVFYMPGVMTPTELVKAMKLGHTILKLFPGEVVG |
| | PQFVKAMKGPFPNVKFVPTGGVNLDNVAEWFKAGVLAVGVGSALVKGTPDEVREKAKAFVE |
| | KIRGATE |
| BtKY72Rp | RFPNITNLCPFGQVFNASNFPSVYAWERLRISDCVADFAVLYNSSSSFSTFKCYGVSPTKL |
| k9-RBD- | NDLCWSSIYADYFVVKGDDVRQIAPAQTGVIADYNYKLPDDFTGCVLAWNTNSVDSKSGNN |
| I53-50A | FYYRLFRHGKIKPYERDISNVLYNSAGGTCSSISQLGCYEPLKSYGFTPTVGVGYQPYRVV |
| SEQ ID | VLSFELLNAPATVCGPKKSTGGSGGSGSGGSGGSGSEKAAKAEEAARKMEELFKKHKIVAV |
| NO: 35 | LRANSVEEAIEKAVAVFAGGVHLIEITFTVPDADTVIKALSVLKEKGAIIGAGTVTSVEQA |
| | RKAVESGAEFIVSPHLDEEISQFAKEKGVFYMPGVMTPTELVKAMKLGHTILKLFPGEVVG |
| | PQFVKAMKGPFPNVKFVPTGGVNLDNVAEWFKAGVLAVGVGSALVKGTPDEVREKAKAFVE |
| | KIRGATE |
| PRD0038- | RFPNITNLCPFGQVFNASKEPSVYAWERLRISDCVADYSVLYNSSSSFSTFKCYGVSPTKL |
| RBD-I53- | NDLCFSSVYADYFVVKGDDVRQIAPAQTGVIADYNYKLPDDFTGCVLAWNTNSVDSKQGNN |
| 50A | FYYRLFRHGKIKPYERDISNVLYNSAGGTCSSTSQLGCYEPLKSYGFTPTVGVGYQPYRVV |
| SEQ ID | VLSFELLNAPATVCGPKKSTGGSGGSGSGGSGGSGSEKAAKAEEAARKMEELFKKHKIVAV |
| NO: 36 | LRANSVEEAIEKAVAVFAGGVHLIEITFTVPDADTVIKALSVLKEKGAIIGAGTVTSVEQA |
| | RKAVESGAEFIVSPHLDEEISQFAKEKGVFYMPGVMTPTELVKAMKLGHTILKLFPGEVVG |
| | PQFVKAMKGPFPNVKFVPTGGVNLDNVAEWFKAGVLAVGVGSALVKGTPDEVREKAKAFVE |
| | KIRGATE |
| BM48-31- | RFPNITQLCPFNEVFNITSFPSVYAWERMRITNCVADYSVLYNSSASFSTFQCYGVSPTKL |
| RBD-I53- | NDLCFSSVYADYFVVKGDDVRQIAPAQTGVIADYNYKLPDDFTGCVIAWNTNSLDSSNEFF |
| 50A | YRRFRHGKIKPYGRDLSNVLFNPSGGTCSAEGLNCYKPLASYGFTQSSGIGFQPYRVVVLS |
| SEQ ID | FELLNAPATVCGPKQSTGGSGGSGSGGSGGSGSEKAAKAEEAARKMEELFKKHKIVAVLRA |
| NO: 37 | NSVEEAIEKAVAVFAGGVHLIEITFTVPDADTVIKALSVLKEKGAIIGAGTVTSVEQARKA |
| | VESGAEFIVSPHLDEEISQFAKEKGVFYMPGVMTPTELVKAMKLGHTILKLEPGEVVGPQF |
| | VKAMKGPFPNVKFVPTGGVNLDNVAEWFKAGVLAVGVGSALVKGTPDEVREKAKAFVEKIR |
| | GATE |
| RmYN02- | RFPNITNFCPFDKVFNATRFPNVYAWQRTKISDCIADYTVLYNSTSFSTFKCYGVSPSKLI |
| RBD-I53- | DLCFTSVYADTFLIRFSEVRQIAPGETGVIADYNYKLPDDETGCVLAWNTAQQDIGSYFYR |
| 50A | SHRAVKLKPFERDLSSDENGVRTLSTYDFNPNVPLDYQATRVVVLSFELLNAPATVCGPKL |
| SEQ ID | STGGSGGSGSGGSGGSGSEKAAKAEEAARKMEELFKKHKIVAVLRANSVEEAIEKAVAVFA |
| NO: 38 | GGVHLIEITFTVPDADTVIKALSVLKEKGAIIGAGTVTSVEQARKAVESGAEFIVSPHLDE |
| | EISQFAKEKGVFYMPGVMTPTELVKAMKLGHTILKLFPGEVVGPQFVKAMKGPFPNVKFVP |
| | TGGVNLDNVAEWFKAGVLAVGVGSALVKGTPDEVREKAKAFVEKIRGATE |
| HKU3- | RFPNVYAWERTKISDCVADYTVLYNSTSFSTFKCYGVSPSKLIDLCFTSVYADTFLIRSSE |
| RBD-I53- | VRQVAPGETGVIADYNYKLPDDFTGCVIAWNTAKHDTGNYYYRSHRKTKLKPFERDLSSDD |
| 50A | GNGVYTLSTYDFNPNVPVAYQATRVVVLSFELLNAPATVCGPKLSTGGSGGSGSGGSGGSG |
| SEQ ID | SEKAAKAEEAARKMEELFKKHKIVAVLRANSVEEAIEKAVAVFAGGVHLIEITFTVPDADT |
| NO: 39 | VIKALSVLKEKGAIIGAGTVTSVEQARKAVESGAEFIVSPHLDEEISQFAKEKGVFYMPGV |
| | MTPTELVKAMKLGHTILKLFPGEVVGPQFVKAMKGPFPNVKFVPTGGVNLDNVAEWFKAGV |
| | LAVGVGSALVKGTPDEVREKAKAFVEKIRGATE |
| RsYN04- | RFPSVYAWTRERISNCIADYSVLYNSTSFSTFRCYGVSPTKINDLCFSNVYADSMVVRGDE |
| RBD-I53- | VRQIAPSQTGVIADYNYKLPDDFTGCVIAWNSKAKDENGQYFYRLFRKSKLLPFQRDVSNV |
| 50A | TYGSGKNDGCNPSEADCYWPLLKYGFTSSVSQDYQPYRVVVLSFELLNAPATVCGPKRSTG |
| SEQ ID | GSGGSGSGGSGGSGSEKAAKAEEAARKMEELFKKHKIVAVLRANSVEEAIEKAVAVFAGGV |
| NO: 40 | HLIEITFTVPDADTVIKALSVLKEKGAIIGAGTVTSVEQARKAVESGAEFIVSPHLDEEIS |
| | QFAKEKGVFYMPGVMTPTELVKAMKLGHTILKLFPGEVVGPQFVKAMKGPFPNVKFVPTGG |
| | VNLDNVAEWFKAGVLAVGVGSALVKGTPDEVREKAKAFVEKIRGATE |

In some embodiments, some or all of the fusion proteins comprise a signal peptide at the amino terminus. In this embodiment, any signal peptide may be used as suitable for an intended use. In one non-limiting embodiment the signal peptide comprises the amino acid sequence MGILPSPGMPALLSLVSLLSVLLMGCVAETGT (SEQ ID NO: 87). In various such embodiments, the plurality of fusion proteins comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO:41-61, wherein underlined residues are linkers and may be present or absent, and if present may be substituted with any other linker.

TABLE 5

| | |
|---|---|
| Wu1-RBD- | MGILPSPGMPALLSLVSLLSVLLMGCVAETGT |
| I5350a | RFPNITNLCPFGEVFNATRFASVYAWNRKRISNCVADYSVLYNSASFSTFKCYGVSPTKLNDL |
| SEQ ID | CFTNVYADSFVIRGDEVRQIAPGQTGKIADYNYKLPDDFTGCVIAWNSNNLDSKVGGNYNYLY |
| NO: 41 | RLFRKSNLKPFERDISTEIYQAGSTPCNGVEGFNCYFPLQSYGFQPTNGVGYQPYRVVVLSFE |
| | LLHAPATVCGPKKSTGGSGGSGSGGSGGSGSEKAAKAEEAARKMEELFKKHKIVAVERANSVE |
| | EAIEKAVAVFAGGVHLIEITFTVPDADTVIKALSVLKEKGAIIGAGTVTSVEQARKAVESGAE |
| | FIVSPHLDEEISQFAKEKGVFYMPGVMTPTELVKAMKLGHTILKLFPGEVVGPQFVKAMKGPF |
| | PNVKFVPTGGVNLDNVAEWFKAGVLAVGVGSALVKGTPDEVREKAKAFVEKIRGATE |
| Wu1Rpk9- | MGILPSPGMPALLSLVSLLSVLLMGCVAETGT |
| RBD- | RFPNITNLCPFGEVFNATRFASVYAWNRKRISNCVADFSVLYNSASFSTFKCYGVSPTKLNDL |
| I5350a | CWTNIYADSFVIRGDEVRQIAPGQTGKIADYNYKLPDDFTGCVIAWNSNNLDSKVGGNYNYLY |
| SEQ ID | RLFRKSNLKPFERDISTEIYQAGSTPCNGVEGFNCYFPLQSYGFQPTNGVGYQPYRVVVLSFE |
| NO: 42 | LLHAPATVCGPKKSTGGSGGSGSGGSGGSGSEKAAKAEEAARKMEELFKKHKIVAVLRANSVE |
| | EAIEKAVAVFAGGVHLIEITFTVPDADTVIKALSVLKEKGAIIGAGTVTSVEQARKAVESGAE |
| | FIVSPHLDEEISQFAKEKGVFYMPGVMTPTELVKAMKLGHTILKLEPGEVVGPQFVKAMKGPF |
| | PNVKFVPTGGVNLDNVAEWFKAGVLAVGVGSALVKGTPDEVREKAKAFVEKIRGATE |
| OmicronBA.4/ | MGILPSPGMPALLSLVSLLSVLLMGCVAETGT |
| 5-RBD- | RFPNITNICPFDEVFNATRFASVYAWNRKRISNCVADYSVLYNFAPFFAFKCYGVSPTKLNDL |
| I5350a | CFTNVYADSFVIRGNFVSQIAPGQTGNIADYNYKLPDDFTGCVIAWNSNKLDSKVGGNYNYRY |
| SEQ ID | RLFRKSNLKPFERDISTEIYQAGNKPCNGVAGVNCYFPLQSYGFRPTYGVGHQPYRVVVLSFE |
| NO: 43 | LLHAPATVCGPKKSTGGSGGSGSGGSGGSGSEKAAKAEEAARKMEELFKKHKIVAVLRANSVE |
| | EAIEKAVAVFAGGVHLIEITFTVPDADTVIKALSVLKEKGAIIGAGTVTSVEQARKAVESGAE |
| | FIVSPHLDEEISQFAKEKGVFYMPGVMTPTELVKAMKLGHTILKLFPGEVVGPQFVKAMKGPF |
| | PNVKFVPTGGVNLDNVAEWFKAGVLAVGVGSALVKGTPDEVREKAKAFVEKIRGATE |
| OmicronBA.4/ | MGILPSPGMPALLSLVSLLSVLLMGCVAETGT |
| 5Rpk9- | RFPNITNLCPFDEVFNATRFASVYAWNRKRISNCVADFSVLYNFAPFFAFKCYGVSPTKLNDL |
| RBD- | CWTNIYADSFVIRGNEVSQIAPGQTGNIADYNYKLPDDFTGCVIAWNSNKLDSKVGGNYNYRY |
| I5350a | RLFRKSNLKPFERDISTEIYQAGNKPCNGVAGVNCYFPLQSYGFRPTYGVGHQPYRVVVLSFE |
| SEQ ID | LLHAPATVCGPKKSTGGSGGSGSGGSGGSGSEKAAKAEEAARKMEELFKKHKIVAVLRANSVE |
| NO: 44 | EAIEKAVAVFAGGVHLIEITFTVPDADTVIKALSVLKEKGAIIGAGTVTSVEQARKAVESGAE |
| | FIVSPHLDEEISQFAKEKGVFYMPGVMTPTELVKAMKLGHTILKLFPGEVVGPQFVKAMKGPF |
| | PNVKFVPTGGVNLDNVAEWFKAGVLAVGVGSALVKGTPDEVREKAKAFVEKIRGATE |
| OmicronBA.1- | MGILPSPGMPALLSLVSLLSVLLMGCVAETGT |
| RBD- | RFPNITNLCPFDEVFNATRFASVYAWNRKRISNCVADYSVLYNLAPFFTFKCYGVSPTKLNDL |
| I5350a | CFTNVYADSFVIRGDEVRQIAPGQTGNIADYNYKLPDDFTGCVIAWNSNKLDSKVSGNYNYLY |
| SEQ ID | RLFRKSNLKPFERDISTEIYQAGNKPCNGVAGFNCYFPLRSYSFRPTYGVGHQPYRVVVLSFE |
| NO: 45 | LLHAPATVCGPKKSTGGSGGSGSGGSGGSGSEKAAKAEEAARKMEELFKKHKIVAVLRANSVE |
| | EAIEKAVAVFAGGVHLIEITFTVPDADTVIKALSVLKEKGAIIGAGTVTSVEQARKAVESGAE |
| | FIVSPHLDEEISQFAKEKGVFYMPGVMTPTELVKAMKLGHTILKLFPGEVVGPQFVKAMKGPF |
| | PNVKFVPTGGVNLDNVAEWFKAGVLAVGVGSALVKGTPDEVREKAKAFVEKIRGATE |
| OmicronBA.1 | MGILPSPGMPALLSLVSLLSVLLMGCVAETGT |
| Rpk9- | RFPNITNLCPFDEVFNATRFASVYAWNRKRISNCVADFSVLYNLAPFFTFKCYGVSPTKLNDL |
| RBD- | CWTNIYADSFVIRGDEVRQIAPGQTGNIADYNYKLPDDFTGCVIAWNSNKLDSKVSGNYNYLY |
| I5350a | RLFRKSNLKPFERDISTEIYQAGNKPCNGVAGFNCYFPLRSYSFRPTYGVGHQPYRVVVLSFE |
| SEQ ID | LLHAPATVCGPKKSTGGSGGSGSGGSGGSGSEKAAKAREAARKMEELFKKHKIVAVERANSVE |
| NO: 46 | EAIEKAVAVFAGGVHLIEITFTVPDADTVIKALSVLKEKGAIIGAGTVTSVEQARKAVESGAE |
| | FIVSPHLDEEISQFAKEKGVFYMPGVMTPTELVKAMKLGHTILKLFPGEVVGPQFVKAMKGPF |
| | PNVKFVPTGGVNLDNVAEWFKAGVLAVGVGSALVKGTPDEVREKAKAFVEKIRGATE |
| OmicronBA.2- | MGILPSPGMPALLSLVSLLSVLLMGCVAETGT |
| RBD- | RFPNITNLCPFDEVFNATRFASVYAWNRKRISNCVADYSVLYNFAPFFAFKCYGVSPTKLNDL |
| I5350a | CFTNVYADSFVIRGNEVSQIAPGQTGNIADYNYKLPDDFTGCVIAWNSNKLDSKVGGNYNYLY |
| SEQ ID | RLFRKSNLKPFERDISTEIYQAGNKPCNGVAGFNCYFPLRSYGFRPTYGVGHQPYRVVVLSFE |
| NO: 47 | LLHAPATVCGPKKSTGGSGGSGSGGSGGSGSEKAAKAEEAARKMEELFKKHKIVAVLRANSVE |
| | EAIEKAVAVFAGGVHLIEITFTVPDADTVIKALSVLKEKGAIIGAGTVTSVEQARKAVESGAE |
| | FIVSPHLDEEISQFAKEKGVFYMPGVMTPTELVKAMKLGHTILKLFPGEVVGPQFVKAMKGPF |
| | PNVKFVPTGGVNLDNVAEWFKAGVLAVGVGSALVKGTPDEVREKAKAFVEKIRGATE |
| OmicronBA.2 | MGILPSPGMPALLSLVSLLSVLLMGCVAETGT |
| Rpk9- | RFPNITNLCPFDEVFNATRFASVYAWNRKRISNCVADFSVLYNFAPFFAFKCYGVSPTKLNDL |
| RBD- | CWTNIYADSFVIRGNEVSQIAPGQTGNIADYNYKLPDDFTGCVIAWNSNKLDSKVGGNYNYLY |
| I5350a | RLFRKSNLKPFERDISTEIYQAGNKPCNGVAGFNCYFPLRSYGFRPTYGVGHQPYRVVVLSFE |
| SEQ ID | LLHAPATVCGPKKSTGGSGGSGSGGSGGSGSEKAAKAEEAARKMEELFKKHKIVAVLRANSVE |
| NO: 48 | EAIEKAVAVFAGGVHLIEITFTVPDADTVIKALSVLKEKGAIIGAGTVTSVEQARKAVESGAE |
| | FIVSPHLDEEISQFAKEKGVFYMPGVMTPTELVKAMKLGHTILKLFPGEVVGPQFVKAMKGPF |
| | PNVKFVPTGGVNLDNVAEWFKAGVLAVGVGSALVKGTPDEVREKAKAFVEKIRGATE |
| Delta- | MGILPSPGMPALLSLVSLLSVLLMGCVAETGT |
| RBD- | RFPNITNLCPFGEVFNATRFASVYAWNRKRISNCVADYSVLYNSASFSTFKCYGVSPTKLNDL |
| I5350a | CFTNVYADSFVIRGDEVRQIAPGQTGKIADYNYKLPDDFTGCVIAWNSNNLDSKVGGNYNYRY |
| SEQ ID | RLFRKSNLKPFERDISTEIYQAGSKPCNGVEGFNCYFPLQSYGFQPTNGVGYQPYRVVVLSFE |
| NO: 49 | LLHAPATVCGPKKSTGGSGGSGSGGSGGSGSEKAAKAEEAARKMEELFKKHKIVAVLRANSVE |
| | EAIEKAVAVFAGGVHLIEITFTVPDADTVIKALSVLKEKGAIIGAGTVTSVEQARKAVESGAE |
| | FIVSPHLDEEISQFAKEKGVFYMPGVMTPTELVKAMKLGHTILKLFPGEVVGPQFVKAMKGPF |
| | PNVKFVPTGGVNLDNVAEWEKAGVLAVGVGSALVKGTPDEVREKAKAFVEKIRGATE |
| RaTG13- | MGILPSPGMPALLSLVSLLSVLLMGCVAETGT |
| RBD- | RFPNITNLCPFGEVFNATTFASVYAWNRKRISNCVADYSVLYNSTSFSTFKCYGVSPTKLNDL |
| I5350a | CFTNVYADSEVITGDEVRQIAPGQTGKIADYNYKLPDDFTGCVIAWNSKHIDAKEGGNFNYLY |
| SEQ ID | RLFRKANLKPFERDISTEIYQAGSKPCNGQTGINCYYPLYRYGFYPTDGVGHQPYRVVVLSFE |
| NO: 50 | LLNAPATVCGPKKSTGGSGGSGSGGSGGSGSEKAAKAREAARKMEELFKKHKIVAVLRANSVE |
| | EAIEKAVAVFAGGVHLIEITFTVPDADTVIKALSVLKEKGAIIGAGTVTSVEQARKAVESGAE |
| | FIVSPHLDEEISQFAKEKGVFYMPGVMTPTELVKAMKLGHTILKLEPGEVVGPQFVKAMKGPF |
| | PNVKFVPTGGVNLDNVAEWFKAGVLAVGVGSALVKGTPDEVREKAKAFVEKIRGATE |
| PangolinG | MGILPSPGMPALLSLVSLLSVLLMGCVAETGT |
| X-RBD- | RFPNITNLCPFGEVFNASKFASVYAWNRKRISNCVADYSVLYNSTSFSTFKCYGVSPTKLNDL |
| I5350a | CFTNVYADSFVVKGDEVRQIAPGQTGVIADYNYKLPDDFTGCVIAWNSVKQDALTGGNYLYRL |
| SEQ ID | FRKSKLKPFERDISTETYQAGSTPQNGQVGLNCYYPLERYGFHPTTGVNYQPFRVVVLSFELL |
| NO: 51 | NGPATVCGPKLSTGGSGGSGSGGSGGSGSEKAAKAEEAARKMEELFKKHKIVAVLRANSVEEA |
| | IEKAVAVFAGGVHLIEITFTVPDADTVIKALSVLKEKGAIIGAGTVTSVEQARKAVESGAEFI |
| | VSPHLDEEISQFAKEKGVFYMPGVMTPTELVKAMKLGHTILKLFPGEVVGPQFVKAMKGPFPN |
| | VKFVPTGGVNLDNVAEWFKAGVLAVGVGSALVKGTPDEVREKAKAFVEKIRGATE |
| SARS1- | MGILPSPGMPALLSLVSLLSVLLMGCVAETGT |
| RBD- | RFPNITNLCPFGEVFNATKEPSVYAWERKKISNCVADYSVLYNSTFFSTFKCYGVSATKLNDL |
| I5350a | CFSNVYADSFVVKGDDVRQIAPGQTGVIADYNYKLPDDFMGCVLAWNTRNIDATSTGNYNYKY |
| SEQ ID | RYLRHGKLRPFERDISNVPFSPDGKPCTPPALNCYWPLNDYGFYTTTGIGYQPYRVVVLSFEL |
| NO: 52 | LNAPATVCGPKLSTGGSGGSGSGGSGGSGSEKAAKAEEAARKMEELFKKHKIVAVLRANSVEE |
| | AIEKAVAVFAGGVHLIEITFTVPDADTVIKALSVLKEKGAIIGAGTVTSVEQARKAVESGAEF |
| | IVSPHLDEEISQFAKEKGVFYMPGVMTPTELVKAMKLGHTILKLFPGEVVGPQFVKAMKGPFP |
| | NVKFVPTGGVNLDNVAEWFKAGVLAVGVGSALVKGTPDEVREKAKAFVEKIRGATE |
| SHC014- | MGILPSPGMPALLSLVSLLSVLLMGCVAETGT |
| RBD- | RFPNITNLCPFGEVFNATTFPSVYAWERKRISNCVADYSVLYNSTSFSTFKCYGVSATKLNDL |
| I5350a | CFSNVYADSFVVKGDDVRQIAPGQTGVIADYNYKLPDDFLGCVLAWNTNSKDSSTSGNYNYLY |
| SEQ ID | RWVRRSKLNPYERDLSNDIYSPGGQSCSAVGPNCYNPLRPYGFFTTAGVGHQPYRVVVLSFEL |
| NO: 53 | LNAPATVCGPKLSTGGSGGSGSGGSGGSGSEKAAKAEEAARKMEELFKKHKIVAVLRANSVEE |
| | AIEKAVAVFAGGVHLIEITFTVPDADTVIKALSVLKEKGAIIGAGTVTSVEQARKAVESGAEF |
| | IVSPHLDEEISQFAKEKGVFYMPGVMTPTELVKAMKLGHTILKLFPGEVVGPQFVKAMKGPFP |
| | NVKFVPTGGVNLDNVAEWEKAGVLAVGVGSALVKGTPDEVREKAKAFVEKIRGATE |
| WIV1/16- | MGILPSPGMPALLSLVSLLSVLLMGCVAETGT |
| RBD- | RFPNITNLCPFGEVFNATTFPSVYAWERKRISNCVADYSVLYNSTSFSTFKCYGVSATKLNDL |
| I5350a | CFSNVYADSFVVKGDDVRQIAPGQTGVIADYNYKLPDDFTGCVLAWNTRNIDATQTGNYNYKY |
| SEQ ID | RSLRHGKLRPFERDISNVPFSPDGKPCTPPAFNCYWPLNDYGFYITNGIGYQPYRVVVLSFEL |
| NO: 54 | LNAPATVCGPKLSTGGSGGSGSGGSGGSGSEKAAKAEEAARKMEELFKKHKIVAVLRANSVEE |
| | AIEKAVAVFAGGVHLIEITFTVPDADTVIKALSVLKEKGAIIGAGTVTSVEQARKAVESGAEF |
| | IVSPHLDEEISQFAKEKGVFYMPGVMTPTELVKAMKLGHTILKLFPGEVVGPQFVKAMKGPFP |
| | NVKFVPTGGVNLDNVAEWFKAGVLAVGVGSALVKGTPDEVREKAKAFVEKIRGATE |
| BtKY71- | MGILPSPGMPALLSLVSLLSVLLMGCVAETGT |
| RBD- | RFPNITNLCPFGQVFNASNFPSVYAWERLRISDCVADYAVLYNSSSSFSTFKCYGVSPTKLND |
| I5350a | LCFSSVYADYFVVKGDDVRQIAPAQTGVIADYNYKLPDDFTGCVLAWNTNSVDSKSGNNFYYR |
| SEQ ID | LFRHGKIKPYERDISNVLYNSAGGTCSSISQLGCYEPLKSYGFTPTVGVGYQPYRVVVLSFEL |
| NO: 55 | LNAPATVCGPKKSTGGSGGSGSGGSGGSGSEKAAKAEEAARKMEELFKKHKIVAVLRANSVEE |
| | AIEKAVAVFAGGVHLIEITFTVPDADTVIKALSVLKEKGAIIGAGTVTSVEQARKAVESGAEF |
| | IVSPHLDEEISQFAKEKGVFYMPGVMTPTELVKAMKLGHTILKLFPGEVVGPQFVKAMKGPFP |
| | NVKFVPTGGVNLDNVAEWFKAGVLAVGVGSALVKGTPDEVREKAKAFVEKIRGATE |
| BtKY72Rpk | MGILPSPGMPALLSLVSLLSVLLMGCVAETGT |
| 9-RBD- | RFPNITNLCPFGQVFNASNFPSVYAWERLRISDCVADFAVLYNSSSSFSTFKCYGVSPTKLND |
| I5350a | LCWSSIYADYFVVKGDDVRQIAPAQTGVIADYNYKLPDDFTGCVLAWNTNSVDSKSGNNFYYR |
| SEQ ID | LFRHGKIKPYERDISNVLYNSAGGTCSSISQLGCYEPLKSYGFTPTVGVGYQPYRVVVLSFEL |
| NO: 56 | LNAPATVCGPKKSTGGSGGSGSGGSGGSGSEKAAKAEEAARKMEELEKKHKIVAVLRANSVEE |
| | AIEKAVAVFAGGVHLIEITFTVPDADTVIKALSVLKEKGAIIGAGTVTSVEQARKAVESGAEF |
| | IVSPHLDEEISQFAKEKGVFYMPGVMTPTELVKAMKLGHTILKLFPGEVVGPQFVKAMKGPFP |
| | NVKFVPTGGVNLDNVAEWFKAGVLAVGVGSALVKGTPDEVREKAKAFVEKIRGATE |
| PRD0038- | MGILPSPGMPALLSLVSLLSVLLMGCVAETGT |
| RBD- | RFPNITNLCPFGQVFNASKFPSVYAWERLRISDCVADYSVLYNSSSSFSTFKCYGVSPTKLND |
| I5350a | LCFSSVYADYFVVKGDDVRQIAPAQTGVIADYNYKLPDDFTGCVLAWNTNSVDSKQGNNFYYR |
| SEQ ID | LFRHGKIKPYERDISNVLYNSAGGTCSSTSQLGCYEPLKSYGFTPTVGVGYQPYRVVVLSFEL |
| NO: 57 | LNAPATVCGPKKSTGGSGGSGSGGSGGSGSEKAAKAEEAARKMEELFKKHKIVAVLRANSVEE |
| | AIEKAVAVFAGGVHLIEITFTVPDADTVIKALSVLKEKGAIIGAGTVTSVEQARKAVESGAEF |
| | IVSPHLDEEISQFAKEKGVFYMPGVMTPTELVKAMKLGHTILKLFPGEVVGPQFVKAMKGPFP |
| | NVKFVPTGGVNLDNVAEWFKAGVLAVGVGSALVKGTPDEVREKAKAFVEKIRGATE |
| BM48-31- | MGILPSPGMPALLSLVSLLSVLLMGCVAETGT |
| RBD- | RFPNITQLCPFNEVFNITSFPSVYAWERMRITNCVADYSVLYNSSASFSTFQCYGVSPTKLND |
| I5350a | LCFSSVYADYFVVKGDDVRQIAPAQTGVIADYNYKLPDDFTGCVIAWNTNSLDSSNEFFYRRF |
| SEQ ID | RHGKIKPYGRDLSNVLFNPSGGTCSAEGLNCYKPLASYGFTQSSGIGFQPYRVVVLSFELLNA |
| NO: 58 | PATVCGPKQSTGGSGGSGSGGSGGSGSEKAAKAEEAARKMEELFKKHKIVAVLRANSVEEAIE |
| | KAVAVFAGGVHLIEITFTVPDADTVIKALSVLKEKGAIIGAGTVTSVEQARKAVESGAEFIVS |
| | PHLDEEISQFAKEKGVFYMPGVMTPTELVKAMKLGHTILKLFPGEVVGPQFVKAMKGPFPNVK |
| | FVPTGGVNLDNVAEWFKAGVLAVGVGSALVKGTPDEVREKAKAFVEKIRGATE |
| RmYN02- | MGILPSPGMPALLSLVSLLSVLLMGCVAETGT |
| RBD- | RFPNITNFCPFDKVFNATRFPNVYAWQRTKISDCIADYTVLYNSTSFSTFKCYGVSPSKLIDL |
| I5350a | CFTSVYADTFLIRFSEVRQIAPGETGVIADYNYKLPDDFTGCVLAWNTAQQDIGSYFYRSHRA |
| SEQ ID | VKLKPFERDLSSDENGVRTLSTYDFNPNVPLDYQATRVVVLSFELLNAPATVCGPKLSTGGSG |
| NO: 59 | GSGSGGSGGSGSEKAAKAEEAARKMEELFKKHKIVAVLRANSVEEAIEKAVAVFAGGVHLIEI |
| | TFTVPDADTVIKALSVLKEKGAIIGAGTVTSVEQARKAVESGAEFIVSPHLDEEISQFAKEKG |
| | VFYMPGVMTPTELVKAMKLGHTILKLFPGEVVGPQFVKAMKGPFPNVKFVPTGGVNLDNVAEW |
| | FKAGVLAVGVGSALVKGTPDEVREKAKAFVEKIRGATE |
| HKU3-RBD- | MGILPSPGMPALLSLVSLLSVLLMGCVAETGT |
| I5350a | RFPNVYAWERTKISDCVADYTVLYNSTSFSTFKCYGVSPSKLIDLCFTSVYADTFLIRSSEVR |
| SEQ ID | QVAPGETGVIADYNYKLPDDFTGCVIAWNTAKHDTGNYYYRSHRKTKLKPFERDLSSDDGNGV |
| NO: 60 | YTLSTYDFNPNVPVAYQATRVVVLSFELLNAPATVCGPKLSTGGSGGSGSGGSGGSGSEKAAK |
| | AEEAARKMEELFKKHKIVAVLRANSVEEAIEKAVAVFAGGVHLIEITFTVPDADTVIKALSVL |
| | KEKGAIIGAGTVTSVEQARKAVESGAEFIVSPHLDEEISQFAKEKGVFYMPGVMTPTELVKAM |
| | KLGHTILKLFPGEVVGPQFVKAMKGPFPNVKFVPTGGVNLDNVAEWFKAGVLAVGVGSALVKG |
| | TPDEVREKAKAFVEKIRGATE |
| RsYN04- | MGILPSPGMPALLSEVSLLSVLLMGCVAETGT |
| RBD- | RFPSVYAWTRERISNCIADYSVLYNSTSFSTFRCYGVSPTKINDLCFSNVYADSMVVRGDEVR |
| I5350a | QIAPSQTGVIADYNYKLPDDFTGCVIAWNSKAKDENGQYFYRLFRKSKLLPFQRDVSNVTYGS |
| SEQ ID | GKNDGCNPSEADCYWPLLKYGFTSSVSQDYQPYRVVVLSFELLNAPATVCGPKRSTGGSGGSG |
| NO: 61 | SGGSGGSGSEKAAKAEEAARKMEELFKKHKIVAVLRANSVEEAIEKAVAVFAGGVHLIEITFT |
| | VPDADTVIKALSVLKEKGAIIGAGTVTSVEQARKAVESGAEFIVSPHLDEEISQFAKEKGVFY |
| | MPGVMTPTELVKAMKLGHTILKLFPGEVVGPQFVKAMKGPFPNVKFVPTGGVNLDNVAEWFKA |
| | GVLAVGVGSALVKGTPDEVREKAKAFVEKIRGATE |

The fusion proteins may further comprise any other domains as appropriate for an intended use, including purification tags. In various such embodiments, the plurality of fusion proteins comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO: 62-82.

TABLE 6

| | |
|---|---|
| Wu1-RBD- | MGILPSPGMPALLSLVSLLSVLLMGCVAETGTRFPNITNLCPFGEVFNATRFASVYAWNRKRIS |
| I5350a | NCVADYSVLYNSASFSTFKCYGVSPTKLNDLCFTNVYADSFVIRGDEVRQIAPGQTGKIADYNY |
| SEQ ID | KLPDDFTGCVIAWNSNNLDSKVGGNYNYLYRLFRKSNLKPFERDISTEIYQAGSTPCNGVEGFN |
| NO: 62 | CYFPLQSYGFQPTNGVGYQPYRVVVLSFELLHAPATVCGPKKSTGGSGGSGSGGSGGSGSEKAA |
| | KAEEAARKMEELFKKHKIVAVLRANSVEEAIEKAVAVFAGGVHLIEITFTVPDADTVIKALSVL |
| | KEKGAIIGAGTVTSVEQARKAVESGAEFIVSPHLDEEISQFAKEKGVFYMPGVMTPTELVKAMK |
| | LGHTILKLFPGEVVGPQFVKAMKGPFPNVKFVPTGGVNLDNVAEWFKAGVLAVGVGSALVKGTP |
| | DEVREKAKAFVEKIRGATEGGSHHHHHHHH |
| Wu1Rpk9- | MGILPSPGMPALLSLVSLLSVLLMGCVAETGTRFPNITNLCPFGEVFNATRFASVYAWNRKRIS |
| RBD- | NCVADFSVLYNSASFSTFKCYGVSPTKLNDLCWTNIYADSFVIRGDEVRQIAPGQTGKIADYNY |
| I5350a | KLPDDFTGCVIAWNSNNLDSKVGGNYNYLYRLFRKSNLKPFERDISTEIYQAGSTPCNGVEGFN |
| SEQ ID | CYFPLQSYGFQPTNGVGYQPYRVVVLSFELLHAPATVCGPKKSTGGSGGSGSGGSGGSGSEKAA |
| NO: 63 | KAEEAARKMEELFKKHKIVAVLRANSVEEAIEKAVAVFAGGVHLIEITFTVPDADTVIKALSVL |
| | KEKGAIIGAGTVTSVEQARKAVESGAEFIVSPHLDEEISQFAKEKGVFYMPGVMTPTELVKAMK |
| | LGHTILKLFPGEVVGPQFVKAMKGPFPNVKFVPTGGVNLDNVAEWFKAGVLAVGVGSALVKGTP |
| | DEVREKAKAFVEKIRGATEGGSHHHHHHHH |
| OmicronBA.4/ | MGILPSPGMPALLSLVSLLSVLLMGCVAETGTRFPNITNLCPFDEVFNATRFASVYAWNRKRIS |
| 5-RBD- | NCVADYSVLYNFAPFFAFKCYGVSPTKLNDLCFTNVYADSFVIRGNEVSQIAPGQTGNIADYNY |
| I5350a | KLPDDFTGCVIAWNSNKLDSKVGGNYNYRYRLFRKSNLKPFERDISTEIYQAGNKPCNGVAGVN |
| SEQ ID | CYFPLQSYGFRPTYGVGHQPYRVVVLSFELLHAPATVCGPKKSTGGSGGSGSGGSGGSGSEKAA |
| NO: 64 | KAEEAARKMEELFKKHKIVAVLRANSVEEAIEKAVAVFAGGVHLIEITFTVPDADTVIKALSVL |
| | KEKGAIIGAGTVTSVEQARKAVESGAEFIVSPHLDEEISQFAKEKGVFYMPGVMTPTELVKAMK |
| | LGHTILKLFPGEVVGPQFVKAMKGPFPNVKFVPTGGVNLDNVAEWFKAGVLAVGVGSALVKGTP |
| | DEVREKAKAFVEKIRGATEGGSHHHHHHHH |
| OmicronBA.4/ | MGILPSPGMPALLSLVSLLSVLLMGCVAETGTRFPNITNLCPFDEVFNATRFASVYAWNRKRIS |
| 5Rpk9- | NCVADFSVLYNFAPFFAFKCYGVSPTKLNDLCWTNIYADSFVIRGNEVSQIAPGQTGNIADYNY |
| RBD- | KLPDDFTGCVIAWNSNKLDSKVGGNYNYRYRLFRKSNLKPFERDISTEIYQAGNKPCNGVAGVN |
| I5350a | CYFPLQSYGFRPTYGVGHQPYRVVVLSFELLHAPATVCGPKKSTGGSGGSGSGGSGGSGSEKAA |
| SEQ ID | KAEEAARKMEELFKKHKIVAVLRANSVEEAIEKAVAVFAGGVHLIEITFTVPDADTVIKALSVL |
| NO: 65 | KEKGAIIGAGTVTSVEQARKAVESGAEFIVSPHLDEEISQFAKEKGVFYMPGVMTPTELVKAMK |
| | LGHTILKLFPGEVVGPQFVKAMKGPFPNVKFVPTGGVNLDNVAEWFKAGVLAVGVGSALVKGTP |
| | DEVREKAKAFVEKIRGATEGGSHHHHHHHH |
| OmicronBA.1- | MGILPSPGMPALLSLVSLLSVLLMGCVAETGTRFPNITNLCPFDEVFNATREASVYAWNRKRIS |
| RBD- | NCVADYSVLYNLAPFFTFKCYGVSPTKLNDLCFTNVYADSFVIRGDEVRQIAPGQTGNIADYNY |
| I5350a | KLPDDFTGCVIAWNSNKLDSKVSGNYNYLYRLFRKSNLKPFERDISTEIYQAGNKPCNGVAGFN |
| SEQ ID | CYFPLRSYSFRPTYGVGHQPYRVVVLSFELLHAPATVCGPKKSTGGSGGSGSGGSGGSGSEKAA |
| NO: 66 | KAEEAARKMEELFKKHKIVAVERANSVEEAIEKAVAVFAGGVHLIEITFTVPDADTVIKALSVL |
| | KEKGAIIGAGTVTSVEQARKAVESGAEFIVSPHLDEEISQFAKEKGVFYMPGVMTPTELVKAMK |
| | LGHTILKLFPGEVVGPQFVKAMKGPFPNVKFVPTGGVNLDNVAEWFKAGVLAVGVGSALVKGTP |
| | DEVREKAKAFVEKIRGATEGGSHHHHHHHH |
| OmicronBA.1 | MGILPSPGMPALLSLVSLLSVLLMGCVAETGTREPNITNLCPFDEVFNATRFASVYAWNRKRIS |
| Rpk9- | NCVADFSVLYNLAPFFTFKCYGVSPTKLNDLCWTNIYADSFVIRGDEVRQIAPGQTGNIADYNY |
| RBD- | KLPDDFTGCVIAWNSNKLDSKVSGNYNYLYRLFRKSNLKPFERDISTEIYQAGNKPCNGVAGFN |
| I5350a | CYFPLRSYSFRPTYGVGHQPYRVVVLSFELLHAPATVCGPKKSTGGSGGSGSGGSGGSGSEKAA |
| SEQ ID | KAEEAARKMEELFKKHKIVAVLRANSVEEAIEKAVAVFAGGVHLIEITFTVPDADTVIKALSVL |
| NO: 67 | KEKGAIIGAGTVTSVEQARKAVESGAEFIVSPHLDEEISQFAKEKGVFYMPGVMTPTELVKAMK |
| | LGHTILKLFPGEVVGPQFVKAMKGPFPNVKFVPTGGVNLDNVAEWFKAGVLAVGVGSALVKGTP |
| | DEVREKAKAFVEKIRGATEGGSHHHHHHHH |
| OmicronBA.2- | MGILPSPGMPALLSLVSLLSVLLMGCVAETGTRFPNITNLCPFDEVFNATRFASVYAWNRKRIS |
| RBD- | NCVADYSVLYNFAPFFAFKCYGVSPTKLNDLCFTNVYADSFVIRGNEVSQIAPGQTGNIADYNY |
| I5350a | KLPDDFTGCVIAWNSNKLDSKVGGNYNYLYRLFRKSNLKPFERDISTEIYQAGNKPCNGVAGFN |
| SEQ ID | CYFPLRSYGFRPTYGVGHQPYRVVVLSFELLHAPATVCGPKKSTGGSGGSGSGGSGGSGSEKAA |
| NO: 68 | KAEEAARKMEELFKKHKIVAVLRANSVEEAIEKAVAVFAGGVHLIEITFTVPDADTVIKALSVL |
| | KEKGAIIGAGTVTSVEQARKAVESGAEFIVSPHLDEEISQFAKEKGVFYMPGVMTPTELVKAMK |
| | LGHTILKLFPGEVVGPQFVKAMKGPFPNVKFVPTGGVNLDNVAEWFKAGVLAVGVGSALVKGTP |
| | DEVREKAKAFVEKIRGATEGGSHHHHHHHH |
| OmicronBA.2 | MGILPSPGMPALLSLVSLLSVLLMGCVAETGTRFPNITNLCPFDEVFNATRFASVYAWNRKRIS |
| Rpk9- | NCVADFSVLYNFAPFFAFKCYGVSPTKLNDLCWTNIYADSFVIRGNEVSQIAPGQTGNIADYNY |
| RBD- | KLPDDFTGCVIAWNSNKLDSKVGGNYNYLYRLFRKSNLKPFERDISTEIYQAGNKPCNGVAGFN |
| I5350a | CYFPLRSYGFRPTYGVGHQPYRVVVLSFELLHAPATVCGPKKSTGGSGGSGSGGSGGSGSEKAA |
| SEQ ID | KAEEAARKMEELFKKHKIVAVLRANSVEEAIEKAVAVFAGGVHLIEITFTVPDADTVIKALSVL |
| NO: 69 | KEKGAIIGAGTVTSVEQARKAVESGAEFIVSPHLDEEISQFAKEKGVFYMPGVMTPTELVKAMK |
| | LGHTILKLFPGEVVGPQFVKAMKGPFPNVKFVPTGGVNLDNVAEWFKAGVLAVGVGSALVKGTP |
| | DEVREKAKAFVEKIRGATEGGSHHHHHHHH |
| Delta- | MGILPSPGMPALLSLVSLLSVLLMGCVAETGTRFPNITNLCPFGEVFNATRFASVYAWNRKRIS |
| RBD- | NCVADYSVLYNSASFSTFKCYGVSPTKLNDLCFTNVYADSFVIRGDEVRQIAPGQTGKIADYNY |
| I5350a | KLPDDFTGCVIAWNSNNLDSKVGGNYNYRYRLFRKSNLKPFERDISTEIYQAGSKPCNGVEGFN |
| SEQ ID | CYFPLQSYGFQPTNGVGYQPYRVVVLSFELLHAPATVCGPKKSTGGSGGSGSGGSGGSGSEKAA |
| NO: 70 | KAEEAARKMEELFKKHKIVAVLRANSVEEAIEKAVAVFAGGVHLIEITFTVPDADTVIKALSVL |
| | KEKGAIIGAGTVTSVEQARKAVESGAEFIVSPHLDEEISQFAKEKGVFYMPGVMTPTELVKAMK |
| | LGHTILKLFPGEVVGPQFVKAMKGPFPNVKFVPTGGVNLDNVAEWFKAGVLAVGVGSALVKGTP |
| | DEVREKAKAFVEKIRGATEGGSHHHHHHHH |
| RaTG13- | MGILPSPGMPALLSLVSLLSVLLMGCVAETGTRFPNITNLCPFGEVFNATTFASVYAWNRKRIS |
| RBD- | NCVADYSVLYNSTSFSTFKCYGVSPTKLNDLCFTNVYADSFVITGDEVRQIAPGQTGKIADYNY |
| I5350a | KLPDDFTGCVIAWNSKHIDAKEGGNFNYLYRLFRKANLKPFERDISTEIYQAGSKPCNGQTGLN |
| SEQ ID | CYYPLYRYGFYPTDGVGHQPYRVVVLSFELLNAPATVCGPKKSTGGSGGSGSGGSGGSGSEKAA |
| NO: 71 | KAEEAARKMEELFKKHKIVAVERANSVEEAIEKAVAVFAGGVHLIEITFTVPDADTVIKALSVL |
| | KEKGAIIGAGTVTSVEQARKAVESGAEFIVSPHLDEEISQFAKEKGVFYMPGVMTPTELVKAMK |
| | LGHTILKLFPGEVVGPQFVKAMKGPFPNVKFVPTGGVNLDNVAEWFKAGVLAVGVGSALVKGTP |
| | DEVREKAKAFVEKIRGATEGGSHHHHHHHH |
| PangolinG | MGILPSPGMPALLSLVSLLSVLLMGCVAETGTRFPNITNLCPFGEVFNASKFASVYAWNRKRIS |
| X-RBD- | NCVADYSVLYNSTSFSTFKCYGVSPTKLNDLCFTNVYADSFVVKGDEVRQIAPGQTGVIADYNY |
| I5350a | KLPDDFTGCVIAWNSVKQDALTGGNYLYRLFRKSKLKPFERDISTEIYQAGSTPCNGQVGLNCY |
| SEQ ID | YPLERYGFHPTTGVNYQPFRVVVLSFELLNGPATVCGPKLSTGGSGGSGSGGSGGSGSEKAAKA |
| NO: 72 | EEAARKMEELFKKHKIVAVLRANSVEEAIEKAVAVFAGGVHLIEITETVPDADTVIKALSVLKE |
| | KGAIIGAGTVTSVEQARKAVESGAEFIVSPHLDEEISQFAKEKGVFYMPGVMTPTELVKAMKLG |
| | HTILKLFPGEVVGPQFVKAMKGPFPNVKFVPTGGVNLDNVAEWFKAGVLAVGVGSALVKGTPDE |
| | VREKAKAFVEKIRGATEGGSHHHHHHHH |
| SARS1- | MGILPSPGMPALLSLVSLLSVLLMGCVAETGTRFPNITNLCPFGEVFNATKEPSVYAWERKKIS |
| RBD- | NCVADYSVLYNSTFFSTFKCYGVSATKLNDLCFSNVYADSFVVKGDDVRQIAPGQTGVIADYNY |
| I5350a | KLPDDFMGCVLAWNTRNIDATSTGNYNYKYRYLRHGKLRPFERDISNVPFSPDGKPCTPPALNC |
| SEQ ID | YWPLNDYGFYTTTGIGYQPYRVVVLSFELLNAPATVCGPKLSTGGSGGSGSGGSGGSGSEKAAK |
| NO: 73 | AEEAARKMEELFKKHKIVAVLRANSVEEAIEKAVAVFAGGVHLIEITFTVPDADTVIKALSVLK |
| | EKGAIIGAGTVTSVEQARKAVESGAEFIVSPHLDEEISQFAKEKGVFYMPGVMTPTELVKAMKL |
| | GHTILKLFPGEVVGPQFVKAMKGPFPNVKFVPTGGVNLDNVAEWFKAGVLAVGVGSALVKGTPD |
| | EVREKAKAFVEKIRGATEGGSHHHHHHHH |
| SHC014- | MGILPSPGMPALLSLVSLLSVLLMGCVAETGTRFPNITNLCPFGEVFNATTFPSVYAWERKRIS |
| RBD- | NCVADYSVLYNSTSFSTFKCYGVSATKLNDLCFSNVYADSFVVKGDDVRQIAPGQTGVIADYNY |
| I5350a | KLPDDFLGCVLAWNTNSKDSSTSGNYNYLYRWVRRSKINPYERDLSNDIYSPGGQSCSAVGPNC |
| SEQ ID | YNPLRPYGFFTTAGVGHQPYRVVVLSFELLNAPATVCGPKLSTGGSGGSGSGGSGGSGSEKAAK |
| NO: 74 | AEEAARKMEELFKKHKIVAVLRANSVEEAIEKAVAVFAGGVHLIEITFTVPDADTVIKALSVLK |
| | EKGAIIGAGTVTSVEQARKAVESGAEFIVSPHLDEEISQFAKEKGVFYMPGVMTPTELVKAMKL |
| | GHTILKLFPGEVVGPQFVKAMKGPFPNVKFVPTGGVNLDNVAEWFKAGVLAVGVGSALVKGTPD |
| | EVREKAKAFVEKIRGATEGGSHHHHHHHH |
| WIV1/16- | MGILPSPGMPALLSLVSLLSVLLMGCVAETGTRFPNITNLCPFGEVFNATTFPSVYAWERKRIS |
| RBD- | NCVADYSVLYNSTSFSTFKCYGVSATKLNDLCFSNVYADSFVVKGDDVRQIAPGQTGVIADYNY |
| I5350a | KLPDDFTGCVLAWNTRNIDATQTGNYNYKYRSLRHGKLRPFERDISNVPFSPDGKPCTPPAFNC |
| SEQ ID | YWPLNDYGFYITNGIGYQPYRVVVLSFELLNAPATVCGPKLSTGGSGGSGSGGSGGSGSEKAAK |
| NO: 75 | AEEAARKMEELFKKHKIVAVERANSVEEAIEKAVAVFAGGVHLIEITFTVPDADTVIKALSVLK |
| | EKGAIIGAGTVTSVEQARKAVESGAEFIVSPHLDEEISQFAKEKGVFYMPGVMTPTELVKAMKL |
| | GHTILKLFPGEVVGPQFVKAMKGPFPNVKFVPTGGVNLDNVAEWFKAGVLAVGVGSALVKGTPD |
| | EVREKAKAFVEKIRGATEGGSHHHHHHHH |
| BtKY72- | MGILPSPGMPALLSLVSLLSVLLMGCVAETGTRFPNITNLCPFGQVFNASNFPSVYAWERLRIS |
| RBD- | DCVADYAVLYNSSSSFSTFKCYGVSPTKLNDLCFSSVYADYFVVKGDDVRQIAPAQTGVIADYN |
| I5350a | YKLPDDFTGCVLAWNTNSVDSKSGNNFYYRLFRHGKIKPYERDISNVLYNSAGGTCSSISQLGC |
| SEQ ID | YEPLKSYGFTPTVGVGYQPYRVVVLSFELLNAPATVCGPKKSTGGSGGSGSGGSGGSGSEKAAK |
| NO: 76 | AEEAARKMEELFKKHKIVAVLRANSVEEAIEKAVAVFAGGVHLIEITFTVPDADTVIKALSVLK |
| | EKGAIIGAGTVTSVEQARKAVESGAEFIVSPHLDEEISQFAKEKGVFYMPGVMTPTELVKAMKL |
| | GHTILKLFPGEVVGPQFVKAMKGPFPNVKFVPTGGVNLDNVAEWFKAGVLAVGVGSALVKGTPD |
| | EVREKAKAFVEKIRGATEGGSHHHHHHHH |
| BtKY72Rpk | MGILPSPGMPALLSEVSLLSVLLMGCVAETGTREPNITNLCPEGQVFNASNFPSVYAWERLRIS |
| 9-RBD- | DCVADFAVLYNSSSSFSTFKCYGVSPTKLNDLCWSSIYADYFVVKGDDVRQIAPAQTGVIADYN |
| I5350a | YKLPDDFTGCVLAWNTNSVDSKSGNNFYYRLFRHGKIKPYERDISNVLYNSAGGTCSSISQLGC |
| SEQ ID | YEPLKSYGFTPTVGVGYQPYRVVVLSFELLNAPATVCGPKKSTGGSGGSGSGGSGGSGSEKAAK |
| NO: 77 | AREAARKMEELFKKAKIVAVERANSVEEAIEKAVAVFAGGVHLIEITFTVPDADTVIKALSVLK |
| | EKGAIIGAGTVTSVEQARKAVESGAEFIVSPHLDEEISQFAKEKGVFYMPGVMTPTELVKAMKL |
| | GHTILKLFPGEVVGPQFVKAMKGPFPNVKFVPTGGVNLDNVAEWFKAGVLAVGVGSALVKGTPD |
| | EVREKAKAFVEKIRGATEGGSHHHHHHHH |
| PRD0038- | MGILPSPGMPALLSLVSLLSVLLMGCVAETGTRFPNITNLCPFGQVFNASKFPSVYAWERLRIS |
| RBD- | DCVADYSVLYNSSSSFSTFKCYGVSPTKLNDLCFSSVYADYFVVKGDDVRQIAPAQTGVIADYN |
| I5350a | YKLPDDFTGCVLAWNTNSVDSKQGNNFYYRLFRHGKIKPYERDISNVLYNSAGGTCSSTSQLGC |
| SEQ ID | YEPLKSYGFTPTVGVGYQPYRVVVLSFELLNAPATVCGPKKSTGGSGGSGSGGSGGSGSEKAAK |
| NO: 78 | AEEAARKMEELFKKHKIVAVLRANSVEEAIEKAVAVFAGGVHLIEITFTVPDADTVIKALSVLK |
| | EKGAIIGAGTVTSVEQARKAVESGAEFIVSPHLDEEISQFAKEKGVFYMPGVMTPTELVKAMKL |
| | GHTILKLFPGEVVGPQFVKAMKGPFPNVKFVPTGGVNLDNVAEWFKAGVLAVGVGSALVKGTPD |
| | EVREKAKAFVEKIRGATEGGSHHHHHHHH |
| BM48-31- | MGILPSPGMPALLSLVSLLSVLLMGCVAETGTRFPNITQLCPFNEVFNITSFPSVYAWERMRIT |
| RBD- | NCVADYSVLYNSSASFSTFQCYGVSPTKLNDLCFSSVYADYFVVKGDDVRQIAPAQTGVIADYN |
| I5350a | YKLPDDFTGCVIAWNTNSLDSSNEFFYRRFRHGKIKPYGRDLSNVLFNPSGGTCSAEGLNCYKP |
| SEQ ID | LASYGFTQSSGIGFQPYRVVVLSFELLNAPATVCGPKQSTGGSGGSGSGGSGGSGSEKAAKAEE |
| NO: 79 | AARKMEELFKKHKIVAVERANSVEEAIEKAVAVFAGGVHLIEITFTVPDADTVIKALSVLKEKG |
| | AIIGAGTVTSVEQARKAVESGAEFIVSPHLDEEISQFAKEKGVFYMPGVMTPTELVKAMKLGHT |
| | ILKLFPGEVVGPQFVKAMKGPFPNVKFVPTGGVNLDNVAEWFKAGVLAVGVGSALVKGTPDEVR |
| | EKAKAFVEKIRGATEGGSHHHHHHHH |
| RmYN02- | MGILPSPGMPALLSLVSLLSVLLMGCVAETGTRFPNITNFCPFDKVFNATRFPNVYAWQRTKIS |
| RBD- | DCIADYTVLYNSTSFSTFKCYGVSPSKLIDLCFTSVYADTFLIRFSEVRQIAPGETGVIADYNY |
| I5350a | KLPDDFTGCVLAWNTAQQDIGSYFYRSHRAVKLKPFERDLSSDENGVRILSTYDFNPNVPLDYQ |
| SEQ ID | ATRVVVLSFELLNAPATVCGPKLSTGGSGGSGSGGSGGSGSEKAAKAEEAARKMEELFKKHKIV |
| NO: 80 | AVLRANSVEEAIEKAVAVFAGGVHLIEITFTVPDADTVIKALSVLKEKGAIIGAGTVTSVEQAR |
| | KAVESGAEFIVSPHLDEEISQFAKEKGVFYMPGVMTPTELVKAMKLGHTILKLFPGEVVGPQFV |
| | KAMKGPFPNVKFVPTGGVNLDNVAEWFKAGVLAVGVGSALVKGTPDEVREKAKAFVEKIRGATE |
| | GGSHHHHHHHH |
| HKU3-RBD- | MGILPSPGMPALLSLVSLLSVLLMGCVAETGTRFPNVYAWERTKISDCVADYTVLYNSTSFSTF |
| I5350a | KCYGVSPSKLIDLCFTSVYADTFLIRSSEVRQVAPGETGVIADYNYKLPDDFTGCVIAWNTAKH |
| SEQ ID | DTGNYYYRSHRKTKLKPFERDLSSDDGNGVYTLSTYDFNPNVPVAYQATRVVVLSFELLNAPAT |
| NO: 81 | VCGPKLSTGGSGGSGSGGSGGSGSEKAAKAEEAARKMEELFKKHKIVAVERANSVEEAIEKAVA |
| | VFAGGVHLIEITFTVPDADTVIKALSVLKEKGAIIGAGTVTSVEQARKAVESGAEFIVSPHLDE |
| | EISQFAKEKGVFYMPGVMTPTELVKAMKLGHTILKLFPGEVVGPQFVKAMKGPFPNVKFVPTGG |
| | VNLDNVAEWFKAGVLAVGVGSALVKGTPDEVREKAKAFVEKIRGATEGGSHHHHHHHH |
| RSYN04- | MGILPSPGMPALLSLVSLLSVLLMGCVAETGTRFPSVYAWTRERISNCIADYSVLYNSTSFSTF |
| RBD- | RCYGVSPTKINDLCFSNVYADSMVVRGDEVRQIAPSQTGVIADYNYKLPDDFTGCVIAWNSKAK |
| I5350a | DENGQYFYRLFRKSKLLPFQRDVSNVTYGSGKNDGCNPSEADCYWPLLKYGFTSSVSQDYQPYR |
| SEQ ID | VVVLSFELLNAPATVCGPKRSTGGSGGSGSGGSGGSGSEKAAKAEEAARKMEELFKKHKIVAVL |
| NO: 82 | RANSVEEAIEKAVAVFAGGVHLIEITFTVPDADTVIKALSVLKEKGAIIGAGTVTSVEQARKAV |
| | ESGAEFIVSPHLDEEISQFAKEKGVFYMPGVMTPTELVKAMKLGHTILKLFPGEVVGPQFVKAM |
| | KGPFPNVKFVPTGGVNLDNVAEWFKAGVLAVGVGSALVKGTPDEVREKAKAFVEKIRGATEGGS |
| | HHHHHHHH |

In one embodiment, the plurality of fusion proteins comprise fusion proteins comprising the amino acid sequence:
(i) selected from the group consisting of SEQ ID NO:20-21, 41-42, and 62-63;
(ii) selected from the group consisting of SEQ ID NO: 31, 52, and 73; and
(iii) selected from the group consisting of SEQ ID NO:35-36, 55-56, and 76-77.

In a further embodiment, the plurality of fusion proteins further comprises fusion proteins comprising the amino acid sequence selected from the group consisting of SEQ ID NO:38, 59, and 80.

In one embodiment, the plurality of fusion proteins comprise fusion proteins having an amino acid sequence at least 80%, 85%, 90%, 91%, 92%, 93%, 94%, 95%, 96%, 97%, 98%, 99%, or 100% identical the amino acid sequence:
(i) selected from the group consisting of SEQ ID NO:20-21, 41-42, and 62-63;
(ii) selected from the group consisting of SEQ ID NO: 31, 52, and 73; and
(iii) selected from the group consisting of SEQ ID NO:35-36, 55-56, and 76-77.

In a further embodiment, the plurality of fusion proteins further comprises fusion proteins comprising the amino acid sequence selected from the group consisting of SEQ ID NO:22-27, 4348, and 64-69. In another embodiment, the plurality of fusion proteins further comprises fusion proteins comprising the amino acid sequence selected from the group consisting of SEQ ID NO:22-23, 43-44, and 64-65.

In other embodiments, the plurality of fusion proteins further comprises fusion proteins comprising the amino acid sequence selected from the group consisting of SEQ ID NO: 38, 59, and 80.

In one embodiment, the nanoparticle comprises a plurality of fusion proteins, wherein the plurality of fusion proteins comprise a first domain comprising an amino acid sequence at least 80%, 85%, 90%, 91%, 92%, 93%, 94%, 95%, 96%, 97%, 98%, 99%, or 100% identical to the amino acid sequence of SEQ ID NO:1, fused to:
(a) an amino acid sequence at least 90%, 91%, 92%, 93%, 94%, 95%, 96%, 97%, 98%, 99%, or 100% identical to the amino acid sequence of SEQ ID NO:5 (clade 1b);
(b) an amino acid sequence at least 90%, 91%, 92%, 93%, 94%, 95%, 96%, 97%, 98%, 99%, or 100% identical to the amino acid sequence of SEQ ID NO:13 (clade 1a); and
(c) an amino acid sequence at least 90%, 91%, 92%, 93%, 94%, 95%, 96%, 97%, 98%, 99%, or 100% identical to the amino acid sequence of SEQ ID NO:16 or 17 (Clade 3);
wherein the N-terminal methionine residue of SEQ ID NO:1 is optional and may be present or absent. In another embodiment, the plurality of fusion proteins further comprise a first domain comprising an amino acid sequence at least 80%, 85%, 90%, 91%, 92%, 93%, 94%, 95%, 96%, 97%, 98%, 99%, or 100% identical to the amino acid sequence of SEQ ID NO:1, fused to:
(d) an amino acid sequence at least 80%, 85%, 90%, 91%, 92%, 93%, 94%, 95%, 96%, 97%, 98%, 99%, or 100% identical to the amino acid sequence of SEQ ID NO:2 (clade 1b);
wherein the N-terminal methionine residue of SEQ ID NO:1 is optional and may be present or absent.

In one embodiment, the nanoparticle comprises a plurality of fusion proteins, wherein the plurality of fusion proteins comprise a first domain comprising the amino acid sequence of SEQ ID NO:1, fused to:
(a) an amino acid sequence at least 80%, 85%, 90%, 91%, 92%, 93%, 94%, 95%, 96%, 97%, 98%, 99%, or 100% identical to the amino acid sequence of SEQ ID NO:5 (clade 1b);
(b) an amino acid sequence at least 80%, 85%, 90%, 91%, 92%, 93%, 94%, 95%, 96%, 97%, 98%, 99%, or 100% identical to the amino acid sequence of SEQ ID NO:13 (clade 1a);
(c) an amino acid sequence at least 80%, 85%, 90%, 91%, 92%, 93%, 94%, 95%, 96%, 97%, 98%, 99%, or 100% identical to the amino acid sequence of SEQ ID NO:16 or 17 (Clade 3); and
(d) an amino acid sequence at least 80%, 85%, 90%, 91%, 92%, 93%, 94%, 95%, 96%, 97%, 98%, 99%, or 100% identical to the amino acid sequence of SEQ ID NO:2 (clade 1b);
wherein the N-terminal methionine residue of SEQ ID NO:1 is optional and may be present or absent. In another embodiment, the plurality of fusion proteins further comprise a fusion protein comprising an amino acid sequence at least 80%, 85%, 90%, 91%, 92%, 93%, 94%, 95%, 96%, 97%, 98%, 99%, or 100% to the amino acid sequence of SEQ ID NO:20 (clade 1b).

In one embodiment, the plurality of fusion proteins comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO:44, 52, 55, and 56. In a further embodiment, the plurality of fusion proteins further comprise a fusion protein comprising an amino acid sequence at least 80%, 85%, 90%, 91%, 92%, 93%, 94%, 95%, 96%, 97%, 98%, 99%, or 100% to the amino acid sequence of SEQ ID NO:41 (clade 1b).

In another embodiment, the plurality of fusion proteins comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO:65, 73, 76, and 77. In a further embodiment, the plurality of fusion proteins further comprise a fusion protein comprising an amino acid sequence at least 80%, 85%, 90%, 91%, 92%, 93%, 94%, 95%, 96%, 97%, 98%, 99%, or 100% to the amino acid sequence of SEQ ID NO:62 (clade 1b).

In a further embodiment, the disclosure provides compositions, comprising a plurality of the nanoparticles of any embodiment or combination of embodiments disclosed above.

The compositions may be used, for example, as mosaic vaccines or therapeutics for use in the methods of the disclosure.

The disclosure also provides compositions that may be used as “cocktail” vaccines or therapeutics, such as in the methods for the disclosure. In one such embodiment, the composition comprises a plurality of nanoparticles, wherein the plurality of nanoparticles comprise:
(a) a first nanoparticle that comprises a fusion protein comprising a first domain comprising an amino acid sequence at least 95%, 96%, 97%, 98%, 99%, or 100% identical to the amino acid sequence of SEQ ID NO:1, fused to an amino acid sequence at least 80%, 85%, 90%, 91%, 92%, 93%, 94%, 95%, 96%, 97%, 98%, 99%, or 100% identical to the amino acid sequence of SEQ ID NO:5 (clade 1b);
(b) a second nanoparticle that comprises a fusion protein comprising a first domain comprising an amino acid sequence at least 95%, 96%, 97%, 98%, 99%, or 100% identical to the amino acid sequence of SEQ ID NO:1, fused to an amino acid sequence at least 80%, 85%, 90%, 91%, 92%, 93%, 94%, 95%, 96%, 97%, 98%, 99%, or 100% identical to the amino acid sequence of SEQ ID NO: 13 (clade 1a); and
(c) a third nanoparticle that comprises a fusion protein comprising a first domain comprising an amino acid sequence at least 95%, 96%, 97%, 98%, 99%, or 100% identical to the amino acid sequence of SEQ ID NO:1, fused to an amino acid sequence at least 80%, 85%, 90%, 91%, 92%, 93%, 94%, 95%, 96%, 97%, 98%, 99%, or 100% identical to the amino acid sequence of SEQ ID NO:16 or 17 (Clade 3);
wherein the N-terminal methionine residue of SEQ ID NO:1 is optional and may be present or absent.

In one embodiment, the composition further comprises a fourth nanoparticle that comprises a fusion protein comprising a first domain comprising an amino acid sequence at least 80%, 85%, 90%, 91%, 92%, 93%, 94%, 95%, 96%, 97%, 98%, 99%, or 100% identical to the amino acid sequence of SEQ ID NO:1, fused to an amino acid sequence at least 80%, 85%, 90%, 91%, 92%, 93%, 94%, 95%, 96%, 97%, 98%, 99%, or 100% identical to the amino acid sequence of SEQ ID NO:2 (clade 1b);
wherein the N-terminal methionine residue of SEQ ID NO:1 is optional and may be present or absent.

In another embodiment, (a) the first nanoparticle comprises a fusion protein comprising an amino acid sequence at least 80%, 85%, 90%, 91%, 92%, 93%, 94%, 95%, 96%, 97%, 98%, 99%, or 100% identical to the amino acid sequence of SEQ ID NO:23;
(b) the second nanoparticle comprises a fusion protein comprising an amino acid sequence at least 80%, 85%, 90%, 91%, 92%, 93%, 94%, 95%, 96%, 97%, 98%, 99%, or 100% identical to the amino acid sequence of SEQ ID NO:31; and
(c) the third nanoparticle comprises a fusion protein comprising an amino acid sequence at least 80%, 85%, 90%, 91%, 92%, 93%, 94%, 95%, 96%, 97%, 98%, 99%, or 100% identical to the amino acid sequence of SEQ ID NO:34 or 35. In one such embodiment, the composition further comprises a fourth nanoparticle comprising a fusion protein comprising an amino acid sequence at least 80%, 85%, 90%, 910, 92%, 93%, 940, 95%, 96%, 97%, 98%, 99%, or 100% to the amino acid sequence of SEQ ID NO:20 (clade 1b).

In one embodiment (a) the first nanoparticle comprises a fusion protein comprising an amino acid sequence at least 80%, 85%, 90%, 91%, 92%, 93%, 94%, 95%, 96%, 97%, 98%, 99%, or 100% identical to the amino acid sequence of SEQ ID NO:44;
(b) the second nanoparticle comprises a fusion protein comprising an amino acid sequence at least 80%, 85%, 90%, 91%, 92%, 93%, 94%, 95%, 96%, 97%, 98%, 99%, or 100% identical to the amino acid sequence of SEQ ID NO:52; and
(c) the third nanoparticle comprises a fusion protein comprising an amino acid sequence at least 80%, 85%, 90%, 91%, 92%, 93%, 94%, 95%, 96%, 97%, 98%, 99%, or 100% identical to the amino acid sequence of SEQ ID NO:55 or 56. In one such embodiment, the composition further comprises a fourth nanoparticle comprising a fusion protein comprising an amino acid sequence at least 80%, 85%, 90%, 91%, 92%, 93%, 94%, 95%, 96%, 97%, 98%, 99%, or 100% to the amino acid sequence of SEQ ID NO:41 (clade 1b).

In another embodiment. (a) the first nanoparticle comprises a fusion protein comprising an amino acid sequence at least 80%, 85%, 90%, 91%, 92%, 93%, 94%, 95%, 96%, 97%, 98%, 99%, or 100% identical to the amino acid sequence of SEQ ID NO:65;
(b) the second nanoparticle comprises a fusion protein comprising an amino acid sequence at least 80%, 85%, 90%, 91%, 92%, 93%, 94%, 950, 96%, 97%, 980, 99%, or 100% identical to the amino acid sequence of SEQ ID NO:73; and
(c) the third nanoparticle comprises a fusion protein comprising an amino acid sequence at least 80%, 85%, 90%, 91%, 92%, 93%, 94%, 95%, 96%, 97%, 98%, 99%, or 100% identical to the amino acid sequence of SEQ ID NO:76 or 77. In one such embodiment, the composition further comprises a fourth nanoparticle comprising a fusion protein comprising an amino acid sequence at least 80%, 85%, 90%, 91%, 92%, 93%, 94%, 95%, 96%, 97%, 98%, 99%, or 100% to the amino acid sequence of SEQ ID NO:62 (clade 1b).

In one embodiment of all these embodiments of the cocktail vaccines or therapeutics of the disclosure, each nanoparticle comprises
(a) a plurality of first assemblies, each first assembly comprising a plurality of identical first proteins comprising an amino acid sequence at least 80%, 85%, 90%, 91%, 92%, 93%, 94%, 95%, 96%, 97%, 98%, 99%, or 100% identical to the amino acid sequence of SEQ ID NO:83-86, or a plurality of first assemblies, each first assembly comprising a plurality of identical first proteins comprising an amino acid sequence at least 80%, 85%, 90%, 91%, 92%, 93%, 94%, 95%, 96%, 97%, 98%, 99%, or 100% identical to the amino acid sequence of SEQ ID NO: 83 or 84; and,
(b) a plurality of second assemblies, each second assembly comprising a plurality of identical fusion proteins as described in the cocktail vaccine/therapeutic embodiments;
wherein the plurality of first assemblies non-covalently interact with the plurality of second assemblies to form the nanoparticle; and
wherein each nanoparticle displays on its surface an immunogenic portion of a sarbecovirus RBD.

In all embodiments of the fusion proteins of the cocktail vaccine/therapeutic, one or more, or all, of the fusion proteins comprise an amino acid linker between the first domain and the RBD domain, as described above.

The disclosure also provides nucleic acids encoding the fusion protein of any embodiment herein, and combinations of nucleic acids encoding the fusion proteins of any embodiment herein. The nucleic acid molecules may comprise RNA (such as mRNA) or DNA. Such nucleic acid molecules may comprise additional sequences useful for promoting expression and/or purification of the encoded protein, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the proteins of the invention. In one embodiment, the nucleic acid molecules comprises mRNAs.

In another embodiment, the disclosure provides expression vectors comprising a nucleic acid molecule of the disclosure operatively linked to a suitable control sequence, and combinations of such expression vectors. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type known in the art, including but not limited to plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive).

The disclosure also provides host cells comprising a fusion protein, nanoparticle, composition, nucleic acid, plurality of nucleic acids, expression vector, and/or one or more expression vector of any embodiment disclosed herein. The cells can be either prokaryotic or eukaryotic, such as mammalian cells. In one embodiment the cells may be transiently or stably transfected with the nucleic acids or expression vectors of the disclosure.

In one embodiment, the disclosure provides pharmaceutical compositions or vaccines comprising
(a) a fusion protein, nanoparticle, composition, nucleic acid, plurality of nucleic acids, one or more expression vector, and/or cell of any embodiment disclosed herein; and
(b) a pharmaceutically acceptable carrier.

The compositions/vaccines may further comprise (a) a lyoprotectant; (b) a surfactant; (c) a bulking agent; (d) a tonicity adjusting agent; (e) a stabilizer; (f) a preservative and/or (g) a buffer. In some embodiments, the buffer in the pharmaceutical composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the nanostructure, in lyophilized or liquid form. Exemplary stabilizers include sucrose, sorbitol, glycine, inositol, sodium chloride, methionine, arginine, and arginine hydrochloride.

In one embodiment, the pharmaceutical composition comprises a plurality of the nanoparticles of any embodiment disclosed herein. In another embodiment, the pharmaceutical composition comprises a plurality of the nucleic acid molecules of any embodiment disclosed herein.

The fusion proteins, nanoparticles, composition, plurality of nucleic acids, one or more expression vector, and/or cell may be the sole active agent in the composition, or the composition may further comprise one or more other agents suitable for an intended use, including but not limited to adjuvants to stimulate the immune system generally and improve immune responses overall. Any suitable adjuvant can be used. The term “adjuvant” refers to a compound or mixture that enhances the immune response to an antigen. Exemplary adjuvants include, but are not limited to, Adju-Phos™, Adjumer™ albumin-heparin microparticles, Algal Glucan, Algammulin, Alum, Antigen Formulation, AS-2 adjuvant, autologous dendritic cells, autologous PBMC, Avridine™, B7-2, BAK, BAY R1005, Bupivacaine, Bupivacaine-HCl, BWZL, Calcitriol, Calcium Phosphate Gel, CCR5 peptides. CFA, Cholera holotoxin (CT) and Cholera toxin B subunit (CTB), Cholera toxin A 1-subunit-Protein A D-fragment fusion protein, CpG, CRL1005, Cytokine-containing Liposomes, D-Murapalmitine, DDA, DHEA, Diphtheria toxoid, DL-PGL, DMPC, DMPG, DOC/Alum Complex, Fowlpox. Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/Ig plasmid, IL-2/Ig protein, IL-4, IL-4 in pcDNA3, Imiquimod™, ImmTher™, Immunoliposomes Containing Antibodies to Costimulatory Molecules, Interferon-gamma, Interleukin-1 beta, Interleukin-12, Interleukin-2, Interleukin-7, ISCOM(s)™, Iscoprep 7.0.3.™, Keyhole Limpet Hemocyanin, Lipid-based Adjuvant, Liposomes, Loxoribine, LT(R192G), LT-OA or LT Oral Adjuvant, LT-R192G, LTK63, LTK72, MF59, MONTANIDE ISA 51, MONTANIDE ISA 720, MPL™, MPL-SE, MTP-PE, MTP-PE Liposomes, Murametide, Murapalmitine, NAGO, nCT native Cholera Toxin, Non-Ionic Surfactant Vesicles, non-toxic mutant E 112K of Cholera Toxin mCT-E 112K, p-Hydroxybenzoique acid methyl ester, pCIL-10, pCIL 12, pCMVmCAT1, pCMVN, Peptomer-NP, Pleuran, PLG, PLGA, PGA, and PLA, Pluronic L121, PMMA, PODDS™, Poly rA:Poly rU, Polysorbate 80, Protein Cochleates, QS-21, Quadri A saponin, Quil-A, Rehydragel HPA, Rehydragel LV, RIBI, Ribilike adjuvant system (MPL, TMD, CWS), S-28463, SAF-1, Sclavo peptide, Sendai Proteoliposomes, Sendai-containing Lipid Matrices, Span 85, Specol, Squalane 1, Squalene 2, Stearyl Tyrosine, Tetanus toxoid (TT), Theramide™, Threonyl muramyl dipeptide (TMDP), Ty Particles, and Walter Reed Liposomes. Selection of an adjuvant depends on the subject to be treated. Preferably, a pharmaceutically acceptable adjuvant is used.

The disclosure also provides methods to treat or limit development of a sarbecovirus infection, comprising administering to a subject in need thereof an amount effective to treat or limit development of the infection of the fusion protein, nanoparticle, composition, nucleic acid, plurality of nucleic acids, one or more expression vector, cell composition, vaccine, and or pharmaceutical composition of any embodiment herein. In one embodiment, the subject is not infected with sarbecovirus, wherein the administering elicits an immune response against sarbecovirus in the subject that limits development of a sarbecovirus infection in the subject.

As used herein, “limiting development” includes, but is not limited to accomplishing one or more of the following: (a) generating an immune response (antibody and/or cell-based) to of sarbecovirus in the subject; (b) generating neutralizing antibodies against sarbecovirus in the subject (b) limiting build-up of sarbecovirus titer in the subject after exposure to sarbecovirus, and/or (c) limiting or preventing development of sarbecovirus symptoms after infection. Exemplary symptoms of sarbecovirus infection include, but are not limited to, fever, fatigue, cough, shortness of breath, chest pressure and/or pain, loss or diminution of the sense of smell, loss or diminution of the sense of taste, and respiratory issues including but not limited to pneumonia, bronchitis, severe acute respiratory syndrome (SARS), and upper and lower respiratory tract infections.

As used herein, an “effective amount” refers to an amount of the immunogenic composition that is effective for treating and/or limiting sarbecovirus infection, fusion proteins, nanoparticles, composition, plurality of nucleic acids, one or more expression vector, cell composition, and or pharmaceutical composition, or vaccine of any embodiment herein are typically formulated as a pharmaceutical composition, such as those disclosed above, and can be administered via any suitable route, including orally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. Polypeptide compositions may also be administered via microspheres, liposomes, immune-stimulating complexes (ISCOMs), or other microparticulate delivery systems or sustained release formulations introduced into suitable tissues (such as blood). Dosage regimens can be adjusted to provide the optimum desired response (e.g., a therapeutic or prophylactic response). A suitable dosage range may, for instance, be 0.1 μg/kg-100 mg/kg body weight of the polypeptide or nanoparticle thereof. The composition can be delivered in a single bolus, or may be administered more than once (e.g., 2, 3, 4, 5, or more times) as determined by attending medical personnel.

The prophylactic or therapeutic composition may be administered as deemed appropriate by attending medical personnel. In one embodiment, the administering comprises administering a first dose and a second dose, wherein the second dose is administered about 2 weeks to about 12 weeks, or about 4 weeks to about 12 weeks the first dose is administered. In various further embodiments, the second dose is administered about 2, 3, 4, 5, 6, 7, 8, 9, 10, 11, or 12 weeks after the first dose. In another embodiment, three doses may be administered, with a second dose administered about 1, 2, 3, 4, 5, 6, 7, 8, 9, 10, 11, or 12 weeks after the first dose, and the third dose administered about 1, 2, 3, 4, 5, 6, 7, 8, 9, 10, 11, or 12 weeks after the second dose.

In some embodiments, the immune response comprises generation of neutralizing antibodies against one or more sarbecoviruses.

In another embodiment, the subject is infected with a sarbecovirus, and the administering elicits an immune response against the sarbecovirus in the subject that treats a sarbecovirus infection in the subject. As used herein, “treat” or “treating” includes, but is not limited to accomplishing one or more of the following: (a) reducing sarbecovirus titer in the subject; (b) limiting any increase of sarbecovirus titer in the subject; (c) reducing the severity of sarbecovirus symptoms; (d) limiting or preventing development of sarbecovirus symptoms after infection; (e) inhibiting worsening of sarbecovirus symptoms; (f) limiting or preventing recurrence of sarbecovirus symptoms in subjects that were previously symptomatic for sarbecovirus infection; and/or (c) survival.

The subject may be any suitable mammalian subject, including but not limited to a human subject.

The disclosure also provides kits, comprising
(a) a plurality of fusion proteins as disclosed in any embodiment herein; and
(b) a protein comprising an amino acid sequence at least 80%, 850, 90%, 91%, 92%, 93%, 94%, 95%, 96%, 97%, 98%, 99%, or 100% identical to the amino acid sequence of SEQ ID NO:83 or 84, wherein residues in parentheses are optional and may be present or absent; and/or
(a1) a plurality of nucleic acid molecules encoding fusion proteins as disclosed in any embodiment herein operatively linked to a suitable control sequence; and
(b1) a nucleic acid encoding a protein comprising an amino acid sequence at least 80%, 85%, 90%, 91%, 92%, 93%, 94%, 95%, 96%, 97%, 98%, 99%, or 100% identical to the amino acid sequence of SEQ ID NO:83 or 84, wherein residues in parentheses are optional and may be present or absent, operatively linked to a suitable control sequence; and/or
(a2) one or more expression vector comprising a plurality of nucleic acid molecules encoding a fusion protein as disclosed in any embodiment herein operatively linked to a suitable control sequence; and
(b2) an expression vector comprising a nucleic acid encoding a protein comprising an amino acid sequence at least 80%, 85%, 90%, 91%, 92%, 93%, 94%, 95%, 96%, 97%, 98%, 99%, or 100% identical to the amino acid sequence of SEQ ID NO:83 or 84, wherein residues in parentheses are optional and may be present or absent, operatively linked to a suitable control sequence; and/or
(a3) a cell comprising one or more expression vector, wherein the one or more expression vector comprises a plurality of nucleic acid molecules encoding a fusion protein as disclosed in any embodiment herein, operatively linked to a suitable control sequence; and
(b3) a cell comprising an expression vector, wherein the expression vector a nucleic acid encoding a protein comprising an amino acid sequence at least 80%, 85%, 90%, 91%, 92%, 93%, 94%, 95%, 96%, 97%, 98%, 99%, or 100% identical to the amino acid sequence of SEQ ID NO:83 or 84, wherein residues in parentheses are optional and may be present or absent, operatively linked to a suitable control sequence.

### EXAMPLES

Recurrent zoonotic sarbecovirus spillovers along with the continued emergence of SARS-CoV-2 variants underscore the need to develop vaccines that elicit broad protection against these pathogens. Using a phylogeny-driven approach, we designed mosaic and cocktail receptor-binding domain vaccines that elicit potent and broad serum neutralizing antibody responses against vaccine-matched strains, including ancestral SARS-CoV-2 and Omicron BA4/5, as well as genetically divergent sarbecoviruses that are not part of the vaccine formulation, including SARS-CoV-2 variants and other clade 3 sarbecoviruses. Immunization of mice with two doses of these vaccines protect against stringent heterotypic challenge with a distant clade 1a bat sarbecovirus (RsSHC014) and boosting mice primed with a clinical SARS-CoV-2 Wu-1 mRNA vaccine induced sera with broad and potent neutralizing activity, motivating clinical development of a lead candidate as a potential pan-variant and pan-sarbecovirus vaccine.

The zoonotic emergence of severe acute respiratory syndrome coronavirus (SARS-CoV) in 2002 and SARS-CoV-2 in 2019 underscores that sarbecoviruses have and will continue to be a threat to global public health. SARS-CoV caused ˜8,000 infections worldwide and ˜800 deaths between 2002 and 2004. The emergence of SARS-CoV-2 in late 2019 resulted in the COVID-19 pandemic that has led to more than 500 million infected individuals and caused over 6 million deaths worldwide. The continued emergence of SARS-CoV-2 variants eroding infection- or vaccine-elicited antibody responses has thus far undermined efforts to end the pandemic. Given the high likelihood of future sarbecovirus spillover events and the limited protection against divergent sarbecoviruses or SARS-CoV-2 variants elicited by current SARS-CoV-2 vaccines, there is an urgent need for innovation and development of broad sarbecovirus countermeasures.

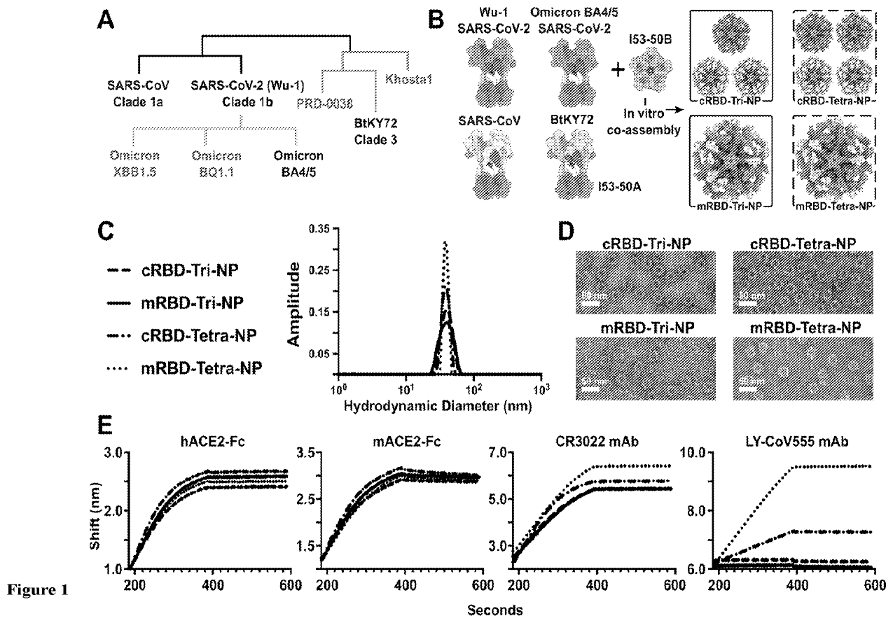

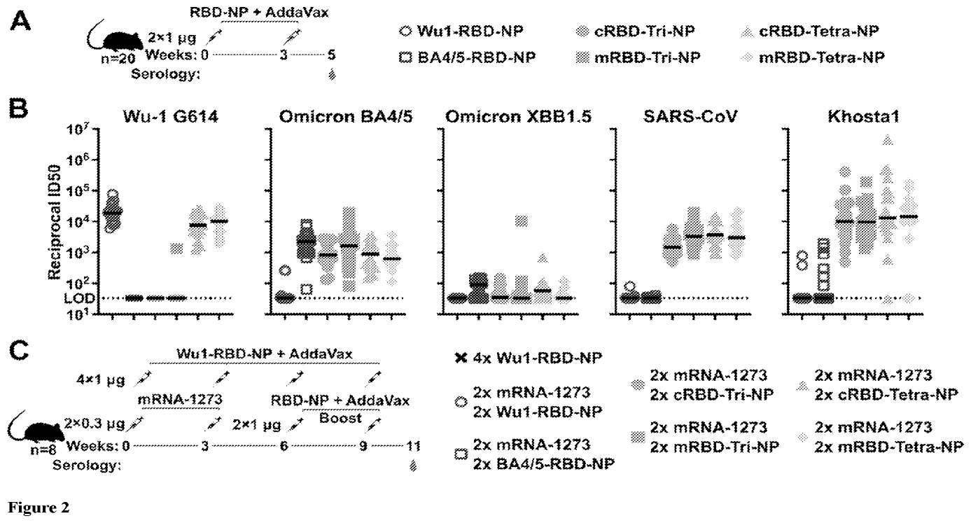

Using a phylogeny-driven approach, we designed next-generation multivalent mosaic nanoparticle vaccine candidates spanning the known sarbecovirus genetic diversity by including one receptor binding domain (RBD) from three of the four major clades, as defined in (1). The rationale for this strategy stems from the potential of mosaic nanoparticle vaccines to maximize elicitation of antibodies targeting conserved antigenic sites by engaging cross-reactive B cell receptors, as recently exemplified by a clinical-stage mosaic nanoparticle vaccine candidate for influenza (2). We expressed and purified the SARS-CoV-2 Wu-1, Omicron BA4/5, SARS-CoV, and BtKY72 RBDs (from clades 1b, 1a, and 3, respectively; FIG. 1A-B) genetically fused to the I53-50A trimer and mixed an equimolar combination of the four trimers with pentameric I53-50B to assemble mosaic (m) RBD nanoparticles (RBD-NP) co-displaying all four RBDs (mRBD-Tetra-NP) or omitted the SARS-CoV-2 Wu-1 RBD (mRBD-Tri-NP; FIG. 1B). We also assembled each antigen bearing component separately into monovalent nanoparticles combining monovalent RBD-NPs together to produce cocktails (c) of RBD-NPs with the same antigenic makeup as the mRBD-NPs (cRBD-Tetra-NP & cRBD-Tri-NP; FIG. 1B). Electron microscopy analysis of negatively stained samples and dynamic light scattering indicated that both nanoparticles adopted the target icosahedral architecture and were homogeneous and monodisperse (FIGS. 1C-D). We confirmed the antigenicity of each RBD-NP immunogen by demonstrating that the SARS-CoV-2 Wu-1. Omicron BA4/5, and SARS-CoV RBDs were recognized by antigen-specific receptors or mAbs using biolayer interferometry (FIG. 1E).

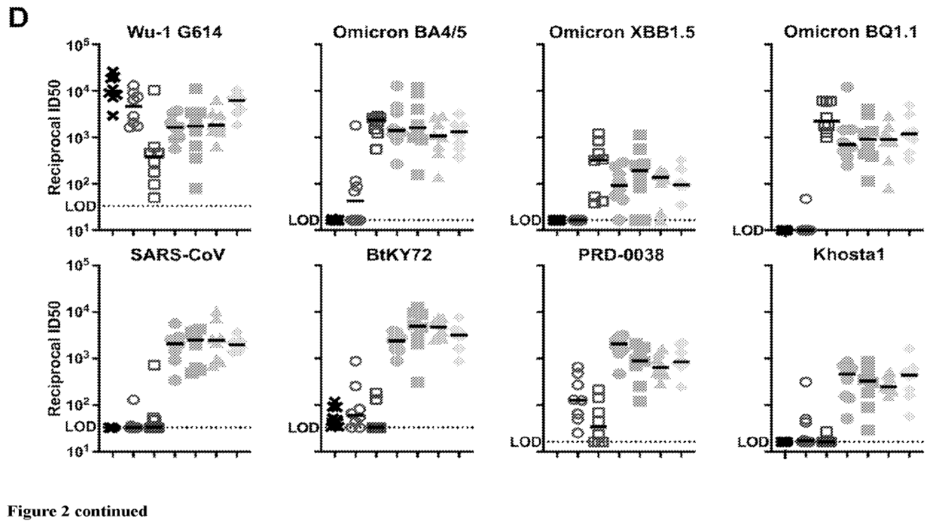

To study neutralizing antibody responses elicited by monovalent RBD-NPs, cRBD-NPs, and mRBD-NPs, we immunized BALB/c mice at weeks 0 and 3 with each immunogen (1 μg total RBD dose) adjuvanted with AddaVax™. Serum was drawn at week 5 (i.e., 2 weeks post-boost) to compare the magnitude and breadth of neutralizing antibodies using single-round vesicular stomatitis virus (VSV) pseudotypes (FIG. 2A). Wu1-RBD-NP elicited the highest geometric mean titer (GMT) of half-maximal inhibitory dilution (ID50) against the vaccine-matched SARS-CoV-2 Wu-1 G614 S VSV (GMT: 1.8×10⁴), as compared to immunogen formulations without the Wu-1 RBD (BA4/5-RBD-NP, cRBD-Tri-NP, and mRBD-Tri-NP) which induced titers below or at the limit of detection (LOD). cRBD-Tetra-NP and mRBD-Tetra-NP induced neutralizing GMTs of 7.5×10³ and 8.6×10³, corresponding to 2.4- and 2.1-fold reductions compared to monovalent Wu1-RBD-NP, respectively (FIG. 2B). Scrum neutralizing GMTs against Omicron BA4/5 VSV S were 8.6×10², 1.4×10³, 7.6×10², and 6.8×10² for cRBD-Tri-NP, mRBD-Tri-NP, cRBD-Tetra-NP and mRBD-Tetra-NP, respectively, amounting to reductions of 2-, 1.3-, 2.4-, and 2.6-fold relative to the monovalent BA4/5-RBD-NP (GMT: 1.8×10³; FIG. 2B). Neutralizing GMTs against Omicron XBB1.5 S VSV, however, revealed that two immunizations with all immunogens was <1×10²(FIG. 2B). The reduction of all RBD-NP-elicited serum neutralizing titers against XBB1.5 relative to vaccine matched G614 or BA4/5 are explained by the 22 mutations present in the RBD of this variant and are consistent with data obtained from individuals who received a primary series of several different vaccines (3-7). After two immunizations, monovalent Wu1-RBD-NP and BA4/5-RBD-NP induced little to no neutralizing activity against sarbecovirus pseudotypes from clade 1a (SARS-CoV) and clade 3 (Khostal) (FIG. 2B). These outcomes mirror data from non-human primates and individuals who received a primary vaccine series (3-5, 8-10) and reflect the marked antigenic differences between these RBDs and the SARS-CoV-2 Wu-1 RBD. By contrast, mice vaccinated with cRBD-Tri-NP, mRBD-Tri-NP, cRBD-Tetra-NP, and mRBD-Tetra-NP had robust serum neutralizing activity against the vaccine-matched SARS-CoV S VSV (GMTs: 1.6×10³, 3.7×10³, 4.1×10³, and 3.3×10³ respectively), which were roughly comparable in potency to those determined against SARS-CoV-2 Wu-1 G614 S VSV (FIG. 2B). cRBD-Tri-NP, mRBD-Tri-NP, cRBD-Tetra-NP, and mRBD-Tetra-NP also elicited potent serum neutralizing activity against vaccine-mismatched Khostal S VSV (GMTs: 1.1×10⁴, 1.1×10⁴, 2.1×10⁴, and 1.1×10⁴; FIG. 2B), which harbors a RBD sharing 88% amino acid sequence identity with BtKY72 (11, 12). Ten mice from each group were then challenged with a lethal mouse adapted divergent clade 1a sarbecovirus RsSHC014 which shares 82% amino acid sequence identity with SARS-CoV (13-15). Mice immunized with cocktail and mosaic formulations of RBD-Tri-NP and RBD-Tetra-NP were protected from the lethal challenge compared to immunogen groups lacking the SARS-CoV RBD (Data not shown). Collectively, these data show that cRBD-Tri-NP, mRBD-Tri-NP, cRBD-Tetra-NP, and mRBD-Tetra-NP perform equivalently well against the pseudoviruses tested and markedly outperform monovalent Wu1-RBD-NP and BA4/5-RBD-NP in terms of broad pseudovirus neutralization and protection against a stringent heterotypic challenge.

We next investigated the breadth of vaccine-elicited antibodies by evaluating serum neutralizing activity, when RBD-NP vaccines are used as boosters in pre-immunized mRNA-1273 (encoding the SARS-CoV-2 Wu-1 S) mice to mimic a hypothetical clinical immunization schedule. We immunized BALB/c mice in 3 week intervals with four immunizations of 1 ug Wu1-RBD-NP adjuvanted with Addavax™ or primed with 2 immunizations of 0.3 ug mRNA-1273 and then boosted with 2 immunizations of 1 μg of monovalent RBD-NPs, cRBD-NPs, or mRBD-NPs adjuvanted with Addavax™ and drew blood two weeks post final immunization (FIG. 2C). All groups elicited neutralizing titers of >1.0×10³ against SARS-CoV-2 Wu-1 G614 S VSV, except those boosted with BA4/5-RBD-NP (GMT: 3.7×10²). GMTs induced by boosting with vaccine-mismatched cRBD-Tri-NP and mRBD-Tri-NP and vaccine-matched cRBD-Tetra-NP were roughly 2.7-, 3.3-, and 2.1-fold less than vaccine-matched boost of monovalent Wu1-RBD-NP (GMTs: 1.6×10³, 1.3×10³, 2×10³, and 4.2×10³, respectively). Only four immunizations of Wu1-RBD-NP and boosting with mRBD-Tetra-NP (GMTs: 1.1×10⁴ and 5.2×10³) elicited GMTs greater than mice boosted with Wu1-RBD-NP against SARS-CoV-2 Wu-1 G614 S VSV (FIG. 2D). However, sera of mice vaccinated four times with Wu1-RBD-NP or boosted with Wu1-RBD-NP did not or poorly neutralized Omicron BA4/5, XBB1.5, and BQ1.1 S VSVs. By contrast, all other immunogens, each containing the Omicron BA4/5 RBD, induced robust levels of neutralizing antibodies against Omicron BA4/5, XBB1.5, and BQ1.1 S VSVs. Sera from mice boosted with BA4/5-RBD-NP had the highest neutralizing activity against the three Omicron subvariants (GMTs: 1.8×10³, 2.1×10², and 2.6×10³, respectively). Cocktail and mosaic formulations of RBD-Tri-NP and RBD-Tetra-NP induced similar levels of antibodies with neutralizing activity to Omicron BA4/5 (GMTs: 9.7×10² to 1.9×10³), XBB1.5 (GMTs: 8.8×10 to 1.4×10²), or BQ1.1 (GMTs: 7.6×10² to 1.2×10³; FIG. 2D).

We then determined the neutralizing ability of sera from mice immunized with mRNA-1273 and boosted with RBD-NPs to a variety of clade 1a and clade 3 sarbecoviruses. Similar to mice immunized with only two doses of c/mRBD-Tri-NPs and c/mRBD-Tetra-NPs (FIG. 2B), mice primed with mRNA-1273 and boosted with c/mRBD-Tri-NPs or c/mRBD-Tetra-NPs produced high neutralizing GMTs against vaccine-matched SARS-CoV S VSV (GMTs: 1.7×10³ to 2.3×10³; FIG. 2D). Comparably, responses induced by vaccine-matched c/mRBD-Tri-NPs and c/m-RBD-Tetra-NPs against BtKY72 DM S VSV remained high across groups (GMTs: 2.1×10³ to 4.5×10³; FIG. 2D). In contrast, monovalent Wu1-RBD-NP and BA4/5-RBD-NP regardless of immunization scheme induced low levels of neutralizing GMTs in sera to vaccine-mismatched clade 1a SARS-CoV and clade 3 BtKY72 DM S VSVs (FIG. 2D). These responses remained comparatively low against more divergent clade 3 sarbecovirus PRD-0038 DM and Khostal S VSVs which have 98% and 88% amino acid sequence identity with BtKY72 RBD, compared to BtKY72 RBD containing immunogens c/mRBD-Tri-NPs and c/mRBD-Tetra-NPs (FIG. 2D) (11, 12, 16). Sera GMTs against PRD-0038 DM were 7- to 13-fold higher (GMTs: 6.9×10² to 1.7×10³) in immunogen groups containing the BtKY72 RBD than those solely boosted with Wu1-RBD-NP (GMT: 1.4×10²) or BA4/5-RBD-NP (GMT: 4.2×10; FIG. 2D). Likewise, neutralizing GMTs against Khostal S VSV were similarly 10- to 11-fold higher for mice boosted with c/mRBD-Tri-NPs and c/mRBD-Tetra-NPs (GMTs: 2.5×10² to 3.4×10²) than the two monovalent RBD-NP boosts (GMTs: 3.1×10 and 1.8×10; FIG. 2D). Cocktail and mosaic formulations of RBD-Tri-NP and RBD-Tetra-NP elicited robust levels of neutralizing antibodies to a diverse array of SARS-CoV-2 variants and sarbecoviruses when used as a boost to an mRNA vaccine encoding the SARS-CoV-2 Wu-1 S.

The emergence of new SARS-CoV-2 variants has clearly outpaced our ability to reformulate vaccines, even with the relatively nimble mRNA technology, indicating that a ‘variant chasing’ approach is not sustainable to control SARS-CoV-2. The recurrent zoonotic spillovers of coronaviruses and the daunting genetic diversity of sarbecoviruses showcase both the need for and the challenges associated with developing countermeasures against these deadly pathogens. Here, mice immunized with two doses of cocktail or mosaic formulations of RBD-Tri-NP (Omicron BA4/5, SARS-CoV, BtKY72) or RBD-Tetra-NP (SARS-CoV-2 Wu-1, Omicron BA4/5, SARS-CoV, BtKY72) were protected from a stringent heterotypic challenge with a divergent clade 1a SARS-CoV-like RsSHC014. We also describe how two doses of mRNA-1273 boosted with two doses of cocktail or mosaic formulations of RBD-Tri-NP (Omicron BA4/5, SARS-CoV, BtKY72) or RBD-Tetra-NP (SARS-CoV-2 Wu-1, Omicron BA4/5, SARS-CoV, BtKY72) developed robust serum neutralizing activity against vaccine-matched sarbecovirus strains, including SARS-CoV-2 Wu-1 and Omicron BA4/5 viruses, and against divergent sarbecoviruses that were not part of the vaccine formulation, including divergent SARS-CoV-2 Omicron variants. These data suggest that a pan-sarbecovirus vaccine may circumvent the need for a pan-variant vaccine.

The magnitude and breadth of neutralizing antibodies elicited by c/mRBD-Tri-NP and c/mRBD-Tetra-NP could be even greater in the context of (i) pre-existing immunity; (ii) hybrid immunity; or (iii) intranasal vaccine delivery. Finally, our results serve as a proof of concept for broadening the mosaic nanoparticle vaccine approach to all betacoronaviruses.

### Methods:

### Cell Lines

Expi293F cells are derived from the HEK293F cell line (Life Technologies). Expi293F cells were grown in Expi293 Expression Medium (Life Technologies), cultured at 36.5° C. with 8% CO₂ and shaking at 150 rpm. HEK293T/17 is a female human embryonic kidney cell line (ATCC). The HEK-ACE2 adherent cell line was obtained through BEI Resources, NIAID, NIH: Human Embryonic Kidney Cells (HEK293T) Expressing Human Angiotensin-Converting Enzyme 2, HEK293T-hACE2 Cell Line, NR-52511. The VeroE6-TMPRSS2 cell line is an African Green monkey kidney cell line expressing TMPRSS2 (17). All adherent cells were cultured at 37° C. with 5% CO₂ in flasks with DMEM+10% FBS (Hyclone)+1% penicillin-streptomycin. Cell lines other than Expi293F were not tested for mycoplasma contamination nor authenticated.

### Mice

Four week-old female BALB/c mice (order code 047, BALB/cAnNHsd strain) were obtained from Envigo, and maintained in a specific pathogen-free facility at the University of Washington, Seattle, WA, accredited by the American Association for the Accreditation of Laboratory Animal Care International (AAALAC). Animal procedures were performed under the approvals of the Institutional Animal Care and Use Committee (IACUC) of University of Washington, Seattle. WA, and University of North Carolina, Chapel Hill, NC.

### Method Details:

### Plasmid Construction

The SARS-CoV-2 Wu-1 (N-RFPN . . . KKST-C), SARS-CoV-2 Omicron BA4/5 (N-RFPN . . . KKST-C). SARS-CoV (N-RFPN . . . KLST-C), and BtKY72 (N-RFPN . . . KKST-C) RBDs were genetically fused to the N terminus of the trimeric I53-50A nanoparticle component using a 16-residue glycine and serine linker. The RBD-16GS-I53-50A fusions were cloned into pCMV/R using the XbaI and AvrII restriction sites using Gibson assembly (18) or by GenScript™. All RBD-bearing components contained an N-terminal mu-phosphatase signal peptide and a C-terminal octa-histidine tag.

### Transient Transfection

Proteins were produced using endotoxin-free DNA in Expi293F cells grown in suspension using Expi293F expression medium (Life Technologies) at 33° C., 70% humidity, 8% CO₂ rotating at 150 rpm. The cultures were transfected using PEI-MAX™ (Polyscience) with cells grown to a density of 3.0 million cells per mL and cultivated for 3 days. Supernatants were clarified by centrifugation (5 min at 4000 ref), addition of PDADMAC solution to a final concentration of 0.0375% (Sigma Aldrich, #409014), and a second centrifugation (5 min at 4000 ref).

### Microbial Plasmid Construction, Protein Expression and Purification of I53-50B.4PT1

I53-50B.4PT1 plasmid was synthesized by GenScript™ in pET29b between the NdeI and XhoI restriction sites with a double-stop codon just before the C-terminal polyhistidine tag. Protein was expressed in Lemo21(DE3) cells (NEB) in LB (10 g Tryptone, 5 g Yeast Extract, 10 g NaCl) grown in a 10 L BioFlo™ 320 Fermenter (Eppendorf). At inoculation, impeller speed was set to 225 rpm, SPLM set to 5 with 02 supplementation as part of the dissolved-oxygen aeration cascade, and the temperature set to 37° C. At the onset of a DO spike (OD˜12), the culture was fed with a bolus addition of 100 mLs of 100% glycerol and induced with 1 mM IPTG. During this time, the culture temperature was reduced to 18° C., and O₂ supplementation was ceased, with expression continuing until an OD˜20. The culture was harvested by centrifugation, and the protein was purified from inclusion bodies. First, pellets were resuspended in PBS, homogenized, and then lysed by microfluidization using a Microfluidics M110P at 18,000 psi using 3 discrete passes. Following sample clarification by centrifugation (24,000×g for 30 min), the supernatant was discarded, and protein was extracted from the inclusion bodies. First, the pellet was washed with PBS, 0.1% Triton X-100, pH 8.0. After this initial wash and sample clarification by centrifugation, the pellet was washed with PBS, 1M NaCl, pH 8.0. Following the second wash, the protein was extracted from the pellet into PBS, 2M urea, 0.75% CHAPs (3-[(3-Cholamidopropyl) dimethylammonio]-1-propanesulfonate), pH 8.0. Following extraction, the sample was applied to a DEAE Sepharose™ FF column (Cytiva) on an AKTA Avant150 FPLC system (Cytiva). After sample binding, the column was washed with 5CV of PBS, 0.1% Triton X-100, pH 8.0, followed by a 5 CV wash with PBS, 0.75% CHAPs, pH 8.0 in series. The protein was eluted with 3 column volumes of PBS, 500 mM NaCl, pH 8.0. After purification, fractions were pooled and concentrated in 10K MWCO centrifugal filters (Millipore), sterile filtered (0.22 μm), and tested to confirm low endotoxin levels before use for nanoparticle assembly.

### Protein Purification

Proteins containing His tags were purified from clarified supernatants via a batch bind method where each clarified supernatant was supplemented with 1 M Tris-HCl pH 8.0 to a final concentration of 45 mM and 5 M NaCl to a final concentration of ˜310 mM. Talon cobalt affinity resin (Takara) or Nickel Sepharose™ Excel resin (Cytiva) were added to the treated supernatants and allowed to incubate for 15 min with gentle shaking. Resin was collected using vacuum filtration with a 0.2 μm filter and transferred to a gravity column. The resin was washed with 20 mM Tris pH 8.0, 300 mM NaCl, and the protein was eluted with 3 column volumes of 20 mM Tris pH 8.0, 300 mM NaCl, 300 mM imidazole. The batch bind process was then repeated and the first and second elutions combined. SDS-PAGE was used to assess purity. RBD-I53-50A fusion protein IMAC elutions were concentrated to >1 mg/mL and subjected to three rounds of dialysis into 50 mM Tris pH 7.4, 185 mM NaCl, 100 mM Arginine, 4.5% glycerol, and 0.75% w/v 3-[(3-cholamidopropyl)dimethylammonio]-1-propanesulfonate (CHAPS) in a hydrated 10K molecular weight cutoff dialysis cassette (Thermo Scientific). Clarified supernatants of cells expressing monoclonal antibodies and mouse or human ACE2-Fc were purified using a MabSelect PrismA™ 2.6×5 cm column (Cytiva) on an AKTA Avant150 FPLC (Cytiva). Bound antibodies were washed with five column volumes of 20 mM NaPO₄, 150 mM NaCl pH 7.2, then five column volumes of 20 mM NaPO₄, 1 M NaCl pH 7.4 and eluted with three column volumes of 100 mM glycine at pH 3.0. The eluate was neutralized with 2 M Trizma base to 50 mM final concentration. SDS-PAGE was used to assess purity.

### In Vitro Nanoparticle Assembly and Purification

Total protein concentration of purified individual nanoparticle components was determined by measuring absorbance at 280 nm using a UV/vis spectrophotometer (Agilent Cary 8454) and calculated extinction coefficients. The assembly steps were performed at room temperature with addition in the following order: RBD-I53-50A trimeric fusion protein, followed by additional buffer (50 mM Tris pH 7.4, 185 mM NaCl, 100 mM Arginine, 4.5% glycerol, and 0.75% w/v CHAPS) as needed to achieve desired final concentration, and finally I53-50B.4PT1 pentameric component (in 50 mM Tris pH 8, 500 mM NaCl, 0.75% w/v CHAPS), with a molar ratio of RBD-I53-50A:I53-50B.4PT1 of 1.1:1. All RBD-I53-50 in vitro assemblies were incubated briefly at room temperature before subsequent purification by SEC in order to remove residual unassembled RBD-I53-50A component. A Superose™ 6 Increase 10/300 GL column was used for nanoparticle purification. Assembled nanoparticles were purified in 50 mM Tris pH 7.4, 185 mM NaCl, 100 mM Arginine, 4.5% glycerol, and 0.75% w/v CHAPS, and elute at ˜11 mL on the Superose™ 6 column. Assembled nanoparticles were sterile filtered (0.22 μm) immediately prior to column application and following pooling of fractions.

### Endotoxin Measurements

Endotoxin levels in protein samples were measured using the EndoSafe™ Nexgen-MCS System (Charles River). Samples were diluted 1:100 in Endotoxin-free LAL reagent water, and applied into wells of an EndoSafe™ LAL reagent cartridge. Charles River EndoScan™-V software was used to analyze endotoxin content, automatically back-calculating for the dilution factor. Endotoxin values were reported as EU/mL which were then converted to EU/mg based on UV/vis measurements. Our threshold for samples suitable for immunization was <50 EU/mg.

### Pseudovirus Production

SARS-CoV-2 Wu-1 G614 S, SARS-CoV-2 Omicron-BA4/5 S, SARS-CoV-2 Omicron-XBB1.5 S, SARS-CoV-2 Omicron-BQ1.1 S, SARS-CoV S (YP 009825051.1), BtKY72-double mutant S with K493Y and T498W (Starr et al 2021), PRD0038-double mutant S with 493Y and 498W and Khostal S pseudotyped VSV viruses were prepared as described previously (19, 20). Briefly, 293T cells in DMEM supplemented with 10% FBS, 1% PenStrep seeded in 10-cm dishes were transfected with the plasmid encoding for the corresponding S glycoprotein using lipofectamine 2000 (Life Technologies) following manufacturer's indications. One day post-transfection, cells were infected with VSV(G*ΔG-luciferase) and after 2 h were washed five times with DMEM before adding medium supplemented with anti-VSV-G antibody (11-mouse hybridoma supernatant, CRL-2700, ATCC). Virus pseudotypes were harvested 18-24 h post-inoculation, clarified by centrifugation at 2,500 g for 5 min, filtered through a 0.45 μm cut off membrane, concentrated 10-fold with a 30 kDa cut off membrane, aliquoted, and stored at −80° C.

### Pseudovirus Neutralization

HEK293-hACE2 cells (BtKY72 DM, PRD-0038 DM, and Khostal) (21) or VeroE6-TMPRSS2 (SARS-CoV-2 Wu-1, Omicron BA4/5, Omicron XBB1.5, Omicron BQ1.1, and SARS-CoV) cells (Lempp et al. 2021) were cultured in DMEM with 10% FBS (Hyclone) and 1% PenStrep with 8% CO₂ in a 37° C. incubator (ThermoFisher). One day prior to infection on HEK-ACE2 cells, 40 μL of poly-lysine (Sigma) was placed into 96-well plates and incubated with rotation for 5 min. Poly-lysine was removed, plates were dried for 5 min then washed 1× with water prior to plating with 40,000 cells. VeroE6-TMPRSS2 cells did not require poly-lysine. The following day, cells were checked to be at 80% confluence. In an empty half-area 96-well plate a 1:3 serial dilution of sera was made in DMEM and diluted VSV pseudovirus was then added to the serial dilution and incubated at room temperature for 30-60 min. After incubation, the sera-virus mixture was added to the cells at 37° C. and 2 hours post-infection, 40 μL 20% FBS-2% PenStrep DMEM was added. After 17-20 h, 40 μL/well of One-Glo-EX substrate (Promega) was added to the cells and incubated in the dark for 5-10 min prior reading on a BioTek™ plate reader. Measurements were done in at least duplicate. Relative luciferase units were plotted and normalized in Prism™ (GraphPad). Nonlinear regression of log(inhibitor) versus normalized response was used to determine IC₅₀ values from curve fits. Kruskal-Wallis tests were used to compare two groups to determine whether they were statistically different.

### Bio-Layer Interferometry (Antigenicity)

Binding of human (h) ACE2-Fc, mouse (m) ACE2-Fc, CR3022 mAb, and LY-CoV555 mAb to RBD-NPs was used to confirm antigenicity of RBDs using an Octet Red™ 96 System (Pall ForteBio/Sartorius) at ambient temperature with shaking at 1000 rpm. Nanoparticles were diluted to 100 nM in Kinetics buffer (1× Kinetics Buffer, 5% w/v milk). Kinetics buffer, receptor or mAb. and nanoparticles were then applied to a black 96-well Greiner Bio-one microplate at 200 μL per well. Protein A biosensors (FortéBio/Sartorius) were first hydrated for 10 minutes in Kinetics buffer, then dipped into receptor or mAb diluted to 10 μg/mL in Kinetics buffer in the immobilization step After 150 seconds, the tips were transferred to Kinetics buffer for 60 seconds to reach a baseline. The receptor or mAb was then loaded with nanoparticle by dipping the loaded biosensors into the immunogens for 200 seconds, and subsequent dissociation in Kinetics buffer for 200 seconds. The data were baseline subtracted prior for plotting using the FortéBio analysis software (version 12.0).

### Negative Stain Electron Microscopy

RBD-NPs were first diluted to 75 μg/mL in 50 mM Tris pH 7.4, 185 mM NaCl, 100 mM Arginine, 4.5% v/v Glycerol, 0.75% w/v CHAPS prior to application of 3 μL of sample onto freshly glow-discharged 300-mesh copper grids. Sample was incubated on the grid for 1 minute before 6 μL of 0.75% w/v uranyl formate stain was applied to the grid. Stain was blotted off with filter paper, then the grids were dipped into another 6 μl of stain and then repeated once more. Finally, the stain was blotted away and the grids were allowed to dry for 1 minute. Prepared grids were imaged in a Talos model LI20C electron microscope at 57,000×.

### Dynamic Light Scattering

Dynamic Light Scattering (DLS) was used to measure hydrodynamic diameter (Dh) and % Polydispersity (% Pd) of RBD-NPs on an UNcle™ Nano-DSF (UNchaned Laboratories). Sample was applied to a 8.8 μL quartz capillary cassette (UNi, UNchained Laboratories) and measured with 10 acquisitions of 5 seconds each, using auto-attenuation of the laser. Increased viscosity due to 4.5% v/v glycerol in the RBD nanoparticle buffer was accounted for by the UNcleT™ Client software in Dh measurements.

### Mouse Immunizations

At fourteen weeks of age, 20 or 8 female BALB/c mice per dosing group were vaccinated with a prime immunization, and every three weeks mice were boosted with subsequent vaccinations according to previously described dosing scheme (FIGS. 2A & 2C) (IACUC protocol 4470.01). Prior to inoculation, RBD-NP immunogen suspensions were gently mixed 1:1 vol/vol with AddaVax™ adjuvant (Invivogen, San Diego, CA) to reach a final concentration of 0.01 mg/mL antigen. Mice were injected intramuscularly into the quadriceps muscle of each hind leg using a 29-gauge 1/2 needle (BD, San Diego, CA) with 50 μL per injection site (100 μL total) of immunogen under isoflurane anesthesia. To obtain sera all mice were bled two weeks after final immunizations. Blood was collected via submental venous puncture and rested in 1.5 mL plastic Eppendorf tubes at room temperature for 30 min to allow for coagulation. Serum was separated from red blood cells via centrifugation at 2,000 g for 10 min. Complement factors and pathogens in isolated serum were heat-inactivated via incubation at 56° C. for 60 min. Serum was stored at 4° C. or −80° C. until use.

### Quantification and Statistical Analysis

Statistical details of experiments can be found in the figure legends. For mouse experiments. 8 or 20 BALB/c animals were used and experiments were completed in at least duplicate. Geometric mean titers were calculated. Kruskal-Wallis tests were performed to compare two groups to determine whether they were statistically different. Significance is indicated with stars: *, p<0.05; ****p<0.0001 and non significant groups are not shown.
1. T. N. Starr, N. Czudnochowski, Z. Liu, F. Zatta, Y.-J. Park, A. Addetia, D. Pinto, M. Beltramello, P. Hemandez, A. J. Greaney, R. Marzi, W. G. Glass, I. Zhang, A. S. Dingens, J. E. Bowen, M. A. Tortorici, A. C. Walls, J. A. Wojcechowskyj, A. De Marco, L. E. Rosen, J. Zhou, M. Montiel-Ruiz, H. Kaiser, J. Dillen, H. Tucker, J. Bassi, C. Silacci-Fregni, M. P. Housley, J. di Iulio, G. Lombardo. M. Agostini, N. Sprugasci, K. Culap, S. Jaconi, M. Meury, E. Dellota, R. Abdelnabi, S.-Y. C. Foo, E. Cameroni, S. Stumpf, T. I. Croll, J. C. Nix, C. Havenar-Daughton, L. Piccoli, F. Benigni, J. Neyts, A. Telenti, F. A. Lempp, M. S. Pizzuto, J. D. Chodera, C. M. Hebner, H. W. Virgin, S. P. J. Whelan, D. Veesler, D. Corti, J. D. Bloom, G. Snell, SARS-CoV-2 RBD antibodies that maximize breadth and resistance to escape. Nature (2021), doi:10.1038/s41586-021-03807-6.
2. S. Boyoglu-Barnum, D. Ellis, R. A. Gillespie. G. B. Hutchinson, Y.-J. Park, S. M. Moin, O. J. Acton, R. Ravichandran, M. Murphy. D. Pettie, N. Matheson, L. Carter, A. Creanga, M. J. Watson, S. Kephart, S. Ataca. J. R. Vaile, G. Ueda, M. C. Crank, L. Stewart, K. K. Lee, M. Guttman, D. Baker, J. R. Mascola, D. Veesler, B. S. Graham, N. P. King, M. Kanekiyo, Quadrivalent influenza nanoparticle vaccines induce broad protection. Nature (2021), doi:10.1038/s41586-021-03365-x.
3. A. C. Walls, K. R. Sprouse, J. E. Bowen, A. Joshi, N. Franko, M. J. Navarro, C. Stewart, E. Cameroni, M. McCallum, E. A. Goecker, E. J. Degli-Angeli, J. Logue, A. Greninger, D. Corti, H. Y. Chu, D. Veesler, SARS-CoV-2 breakthrough infections elicit potent, broad, and durable neutralizing antibody responses. Cell. 185, 872-880.e3 (2022).
4. E. Cameroni, J. E. Bowen, L. E. Rosen, C. Saliba, S. K. Zepeda, K. Culap, D. Pinto, L. A. VanBlargan, A. De Marco, J. di Iulio, F. Zatta, H. Kaiser, J. Noack, N. Farhat, N. Czudnochowski, C. Havenar-Daughton, K. R. Sprouse, J. R. Dillen, A. E. Powell, A. Chen, C. Maher, L. Yin, D. Sun, L. Soriaga, J. Bassi, C. Silacci-Fregni. C. Gustafsson, N. M. Franko, J. Logue, N. T. Iqbal, I. Mazzitelli, J. Geffiner, R. Grifantini, H. Chu, A. Gori, A. Riva, O. Giannini, A. Ceschi, P. Ferrari, P. E. Cippa, A. Franzetti-Pellanda, C. Garzoni, P. J. Halfinann, Y. Kawaoka, C. Hcbner, L. A. Purcell, L. Piccoli, M. S. Pizzuto, A. C. Walls, M. S. Diamond, A. Telenti, H. W. Virgin, A. Lanzavecchia, G. Snell, D. Veesler, D. Corti, Broadly neutralizing antibodies overcome SARS-CoV-2 Omicron antigenic shift. Nature (2021), doi:10.1038/d41586-021-03825-4.
5. J. E. Bowen, K. R. Sprouse, A. C. Walls, I. G. Mazzitelli, J. K. Logue, N. M. Franko, K. Ahmed, A. Shariq, E. Cameroni, A. Gori, A. Bandera, C. M. Posavad, J. M. Dan, Z. Zhang, D. Weiskopf, A. Sette, S. Crotty, N. T. Iqbal, D. Corti, J. Geffner, R. Grifantini, H Y. Chu, D. Veesler, Omicron BA.1 and BA.2 neutralizing activity elicited by a comprehensive panel of human vaccines. bioRxiv (2022), doi:10.1101/2022.03.15.484542.
6. Y. Cao, F. Jian, J. Wang, Y. Yu, W. Song, A. Yisimayi, J. Wang, R. An, X. Chen, N. Zhang, Y. Wang, P. Wang, L. Zhao, H. Sun, L. Yu, S. Yang, X. Niu, T. Xiao, Q. Gu, F. Shao, X. Hao, Y. Xu, R. Jin, Z. Shen, Y. Wang, X. S. Xie, Imprinted SARS-CoV-2 humoral immunity induces convergent Omicron RBD evolution. Nature. 614, 521-529 (2023).
7. A. Addetia, L. Piccoli, J. B. Case, Y.-J. Park, M. Beltramello, B. Guarino, H. Dang, G. D. de Melo, D. Pinto, K. Sprouse, S. M. Scheaffer, J. Bassi, C. Silacci-Fregni, F. Muoio, M. Dini, L. Vincenzetti, R. Acosta, D. Johnson, S. Subramanian. C. Saliba, M. Giurdanella, G. Lombardo, G. Leoni, K. Culap, C. McAlister, A. Rajesh, E. Dellota Jr, J. Zhou, N. Farhat, D. Bohan, J. Noack, A. Chen, F. A. Lempp, J. Quispe, L. Kergoat, F. Larrous, E. Cameroni, B. Whitener, O. Giannini, P. Cippi, A. Ceschi, P. Ferrari, A. Franzetti-Pellanda, M. Biggiogero, C. Garzoni, S. Zappi, L. Bemasconi, M. J. Kim, L. E. Rosen, G. Schnell, N. Czudnochowski, F. Benigni, N. Franko, J. K. Logue, C. Yoshiyama, C. Stewart, H. Chu, H. Bourhy, M. A. Schmid. L. A. Purcell, G. Snell, A. Lanzavecchia, M. S. Diamond, D. Corti, D. Veesler, Neutralization, effector function and immune imprinting of Omicron variants. Nature. 621, 592-601 (2023).
8. A. C. Walls. M. C. Miranda, A. Schifer, M. N. Pham, A. Greaney, P. S. Arunachalam, M.-J. Navarro, M. A. Tortorici, K. Rogers, M. A. O'Connor, L. Shirreff, D. E. Ferrell, J. Bowen, N. Brunette, E. Kepl, S. K. Zepeda, T. Starr, C.-L. Hsieh, B. Fiala, S. Wrenn, D. Pettie, C. Sydeman, K. R. Sprouse, M. Johnson, A. Blackstone. R. Ravichandran, C. Ogohara, L. Carter, S. W. Tilles, R. Rappuoli, S. R. Leist, D. R. Martinez, M. Clark, R. Tisch, D. T. O'Hagan, R. Van Der Most, W. C. Van Voorhis, D. Corti, J. S. McLellan, H. Kleanthous, T. P. Sheahan, K. D. Smith, D. H. Fuller, F. Villinger, J. Bloom, B. Pulendran, R. S. Baric, N. P. King, D. Veesler, Elicitation of broadly protective sarbecovirus immunity by receptor-binding domain nanoparticle vaccines. Cell. 184, 5432-5447.e16 (2021).
9. P. S. Arunachalam, Y. Feng, U. Ashraf. M. Hu, V. V. Edara, V. I. Zarnitsyna. P. P. Aye, N. Golden, K. W. M. Green, B. M. Threeton, N. J. Maness, B. J. Beddingfield, R. P. Bohm, J. Dufour, K. Russell-Lodrigue, M. C. Miranda, A. C. Walls, K. Rogers, L. Shirreff, D. E. Ferrell, N. R. Deb Adhikary, J. Fontenot, A. Grifoni, A. Sette, D. T. O'Hagan, R. Van Der Most, R. Rappuoli, F. Villinger, H. Kleanthous. J. Rappaport, M. S. Suthar, D. Veesler, T. T. Wang, N. P. King, B. Pulendran, Durable protection against SARS-CoV-2 Omicron induced by an adjuvanted subunit vaccine, doi:10.1101/2022.03.18.484950.
10. M. Gagne, J. I. Moliva, K. E. Foulds, S. F. Andrew, B. J. Flynn, A. P. Wemer, D. A. Wagner, I.-T. Teng, B. C. Lin, C. Moore, N. Jean-Baptiste, R. Carroll, S. L. Foster, M. Patel, M. Ellis, V.-V. Edara, N. V. Maldonado, M. Minai, L. McCormick, C. C. Honeycutt, B. M. Nagata, K. W. Bock, C. N. M. Dulan, J. Cordon, D. R. Flebbe, J.-P. M. Todd, E. McCarthy, L. Pessaint, A. Van Ry, B. Narvaez, D. Valentin, A. Cook, A. Dodson, K. Steingrebe, S. T. Nurmukhambetova, S. Godbole, A. R. Henry, F. Laboune, J. Roberts-Torres, C. G. Lorang, S. Amin, J. Trost, M. Naisan, M. Basappa, J. Willis, L. Wang, W. Shi, N. A. Doria-Rose, Y. Zhang, E. S. Yang, K. Leung, S. O'Dell, S. D. Schmidt, A. S. Olia, C. Liu, D. R. Harns, G.-Y. Chuang, G. Stewart-Jones, I. Renzi, Y.-T. Lai, A. Malinowski, K. Wu, J. R. Mascola, A. Carfi, P. D. Kwong, D. K. Edwards, M. G. Lewis, H. Andersen, K. S. Corbett, M. C. Nason, A. B. McDermott. M. S. Suthar, I. N. Moore, M. Roederer, N. J. Sullivan, D. C. Douek, R. A. Seder, mRNA-1273 or mRNA-Omicron boost in vaccinated macaques elicits similar B cell expansion, neutralizing antibodies and protection against Omicron. Cell (2022), doi:10.1016/j.cell.2022.03.038.
11. Y. Tao. S. Tong, Complete Genome Sequence of a Severe Acute Respiratory Syndrome-Related Coronavirus from Kenyan Bats. Microbiol Resour Announc. 8 (2019), doi:10.1128/MRA.00548-19.
12. S. Alkhovsky, S. Lenshin, A. Romashin, T. Vishnevskaya, O. Vyshemirsky, Y. Bulycheva, D. Lvov, A. Gitelman. SARS-like Coronaviruses in Horseshoe Bats (Rhinolophus spp.) in Russia, 2020. Viruses. 14 (2022), doi:10.3390/v14010113.
13. X.-Y. Ge, J.-L. Li, X.-L. Yang, A. A. Chmura, G. Zhu, J. H. Epstein, J. K. Mazet, B. Hu, W. Zhang, C. Peng, Y.-J. Zhang, C.-M. Luo, B. Tan, N. Wang, Y. Zhu, G. Crameri, S.-Y. Zhang, L.-F. Wang, P. Daszak, Z.-L. Shi, Isolation and characterization of a bat SARS-like coronavirus that uses the ACE2 receptor. Nature. 503, 535-538 (2013).
14. P. A. Rota, M. S. Oberste, S. S. Monroe, W. A. Nix, R. Campagnoli, J. P. Icenogle, S. Peilaranda, B. Bankamp, K. Maher, M.-H. Chen, S. Tong, A. Tamin, L. Lowe, M. Frace, J. L. DeRisi, Q. Chen, D. Wang, D. D. Erdman, T. C. T. Peret, C. Bums, T. G. Ksiazek, P. E. Rollin, A. Sanchez, S. Liffick, B. Holloway, J. Limor, K. McCaustland, M. Olsen-Rasmussen, R. Fouchier, S. Gunther, A. D. M. E. Osterhaus, C. Drosten, M. A. Pallansch, L. J. Anderson, W. J. Bellini, Characterization of a novel coronavirus associated with severe acute respiratory syndrome. Science. 300, 1394-1399 (2003).
15. V. D. Menachery, B. L. Yount Jr, K. Debbink, S. Agnihothram, L. E. Gralinski, J. A. Plante, R. L. Graham, T. Scobey, X.-Y. Ge, E. F. Donaldson, S. H. Randell, A. Lanzavecchia, W. A. Marasco, Z.-L. Shi, R S. Baric, A SARS-like cluster of circulating bat coronaviruses shows potential for human emergence. Nat. Med. 21, 1508-1513 (2015).
16. H. L. Wells, M. Letko, G. Lasso, B. Ssebide, J. Nziza, D. K. Byarugaba, I. Navarrete-Macias, E. Liang, M. Cranfield, B. A. Han, M. W. Tingley, M. Diuk-Wasser, T. Goldstein, C. K. Johnson, J. A. K. Mazet, K. Chandran, V. J. Munster, K. Gilardi, S. J. Anthony, The evolutionary history of ACE2 usage within the coronavirus subgenus Sarbecovirus. Virus Evol. 7, veab007 (2021).
17. F. A. Lempp, L. Soriaga. M. Montiel-Ruiz, F. Benigni, J. Noack, Y.-J. Park, S. Bianchi, A. C. Walls, J. E. Bowen, J. Zhou, H. Kaiser, A. Joshi, M. Agostini, M. Meury, E. Dellota Jr, S. Jaconi, E. Cameroni, J. Martinez-Picado, J. Vergara-Alert, N. Izquierdo-Uscros, H. W. Virgin, A. Lanzavecchia, D. Veesler, L. Purcell, A. Telenti, D. Corti, Lectins enhance SARS-CoV-2 infection and influence neutralizing antibodies. Nature (2021), doi:10.1038/s41586-021-03925-1.
18. D. G. Gibson. L. Young, R. Y. Chuang. J. C. Venter, C. A. Hutchison, H. O. Smith, Enzymatic assembly of DNA molecules up to several hundred kilobases. Nat. Methods. 6, 343-345 (2009).
19. M. McCallum, A. De Marco, F. A. Lempp, M. A. Tortorici, D. Pinto, A. C. Walls, M. Beltramello, A. Chen, Z. Liu, F. Zatta, S. Zepeda, J. di Julio, J. E. Bowen, M. Montiel-Ruiz, J. Zhou, L. E. Rosen, S. Bianchi, B. Guarino, C. S. Fregni, R. Abdelnabi, S.-Y. Caroline Foo, P. W. Rothlauf, L.-M. Bloyet, F. Benigni, E. Cameroni, J. Neyts, A. Riva, G. Snell, A. Telenti, S. P. J. Whelan, H. W. Virgin, D. Corti, M. S. Pizzuto, D. Veesler, N-terminal domain antigenic mapping reveals a site of vulnerability for SARS-CoV-2. Cell (2021), doi:10.1016/j.cell.2021.03.028.
20. M. M. Sauer, M. A. Tortorici, Y.-J. Park, A. C. Walls, L. Homad, O. J. Acton, J. E. Bowen, C. Wang, X. Xiong, W. de van der Schueren, J. Quispe, B. G. Hoffstrom, B.-J. Bosch, A. T. McGuire, D. Veesler, Structural basis for broad coronavirus neutralization. Nat. Struct. Mol. Biol. 28, 478-486 (2021).
21. K. H. D. Crawford, R. Eguia, A. S. Dingens, A. N. Loes, K. D. Malone, C. R. Wolf, H. Y. Chu, M. A. Tortorici, D. Veesler, M. Murphy, D. Pettie, N. P. King, A. B. Balazs, J. D. Bloom, Protocol and Reagents for Pseudotyping Lentiviral Particles with SARS-CoV-2 Spike Protein for Neutralization Assays. Viruses. 12 (2020), doi:10.3390/v12050513.

1. A nanoparticle, wherein the nanoparticle displays on its surface an immunogenic portion of clade 1a, clade 1b, and clade 3 sarbecovirus receptor-binding domains (RBD), or variants thereof.

2. The nanoparticle of claim 1, wherein the nanoparticle comprises a plurality of fusion proteins, wherein the plurality of fusion proteins comprise a first domain comprising an amino acid sequence at least 80%, 85%, 90%, 91%, 92%, 93%, 94%, 95%, 96%, 97%, 98%, 99%, or 100% identical to the amino acid sequence of SEQ ID NO:1, fused to an RBD domain comprising:
(a) a clade 1a sarbecovirus RBD,
(ii) a clade 1b sarbecovirus RBD, and
(iii) a clade 3 sarbecovirus RBD;
wherein the N-terminal methionine residue of SEQ ID NO:1 is optional and may be present or absent.

3. The nanoparticle of claim 2, wherein the RBD domains comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO:2-17.

4. The nanoparticle of claim 1, wherein the nanoparticle comprises a plurality of fusion proteins, wherein the plurality of fusion proteins comprise first domain comprising an amino acid sequence at least 80%, 85%, 90%, 91%, 92%, 93%, 94%, 95%, 96%, 97%, 98%, 99%, or 100% identical to the amino acid sequence of SEQ ID NO:1, fused to:
(a) an amino acid at least 80%, 85%, 90%, 91%, 92%, 93%, 94%, 95%, 96%, 97%, 98%, 99%, or 100% identical to the amino acid sequence of SEQ ID NO:2 or 3 (clade 1b);
(b) an amino acid at least 80%, 85%, 90%, 91%, 92%, 93%, 94%, 95%, 96%, 97%, 98%, 99%, or 100% identical to the amino acid sequence of SEQ ID NO:13 (clade 1a); and
(c) an amino acid at least 80%, 85%, 90%, 91%, 92%, 93%, 94%, 95%, 96%, 97%, 98%, 99%, or 100% identical to the amino acid sequence of SEQ ID NO:16 or 17 (Clade 3);
wherein the N-terminal methionine residue of SEQ ID NO:1 is optional and may be present or absent.

5.-9. (canceled)

10. The nanoparticle of claim 2, wherein the plurality of fusion proteins comprise an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO:20-40, wherein optional linker residues may be present or absent, and if present may be substituted with any other linker.

11.-12. (canceled)

13. The nanoparticle of claim 2, wherein the plurality of fusion proteins comprise fusion proteins having an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO:41-61, wherein optional linker residues may be present or absent, and if present may be substituted with any other linker.

14. The nanoparticle of claim 2, wherein the plurality of fusion proteins comprise fusion proteins having an amino acid sequence at least 80%, 85%, 90%, 91%, 92%, 93%, 94%, 95%, 96%, 97%, 98%, 99%, or 100% identical to the amino acid sequence selected from the group consisting of SEQ ID NO: 62-82.

15.-16. (canceled)

17. A nanoparticle comprising a plurality of fusion proteins, wherein the plurality of fusion proteins comprise fusion proteins having an amino acid sequence at least 80%, 85%, 90%, 91%, 92%, 93%, 94%, 95%, 96%, 97%, 98%, 99%, or 100% identical the amino acid sequence:
(i) selected from the group consisting of SEQ ID NO:20-21, 41-42, and 62-63;
(ii) selected from the group consisting of SEQ ID NO: 31, 52, and 73; and
(iii) selected from the group consisting of SEQ ID NO:35-36, 55-56, and 76-77.

18.-19. (canceled)

20. The nanoparticle of claim 1, wherein the nanoparticle comprises a plurality of fusion proteins, wherein the plurality of fusion proteins comprise a first domain comprising an amino acid sequence at least 95%, 96%, 97%, 98%, 99%, or 100% identical to the amino acid sequence of SEQ ID NO:1, fused to:
(a) an amino acid sequence at least 90%, 91%, 92%, 93%, 94%, 95%, 96%, 97%, 98%, 99%, or 100% identical to the amino acid sequence of SEQ ID NO:5 (clade 1b);
(b) an amino acid sequence at least 90%, 91%, 92%, 93%, 94%, 95%, 96%, 97%, 98%, 99%, or 100% identical to the amino acid sequence of SEQ ID NO:13 (clade 1a); and
(c) an amino acid sequence at least 90%, 91%, 92%, 93%, 94%, 95%, 96%, 97%, 98%, 99%, or 100% identical to the amino acid sequence of SEQ ID NO:16 or 17 (Clade 3);
wherein the N-terminal methionine residue of SEQ ID NO:1 is optional and may be present or absent.

21. The nanoparticle of claim 1, wherein the nanoparticle comprises a plurality of fusion proteins, wherein the plurality of fusion proteins comprise a first domain comprising an amino acid sequence at least 98%, 99%, or 100% identical to the amino acid sequence of SEQ ID NO:1, fused to:
(a) an amino acid sequence at least 95%, 96%, 97%, 98%, 99%, or 100% identical to the amino acid sequence of SEQ ID NO:5 (clade 1b);
(b) an amino acid sequence at least 95%, 96%, 97%, 98%, 99%, or 100% identical to the amino acid sequence of SEQ ID NO:13 (clade 1a); and
(c) an amino acid sequence at least 95%, 96%, 97%, 98%, 99%, or 100% identical to the amino acid sequence of SEQ ID NO:16 or 17 (Clade 3);
wherein the N-terminal methionine residue of SEQ ID NO:1 is optional and may be present or absent.

22. The nanoparticle of claim 1, wherein the nanoparticle comprises a plurality of fusion proteins, wherein the plurality of fusion proteins comprise a first domain comprising the amino acid sequence of SEQ ID NO:1, fused to:
(a) an amino acid sequence at least 95%, 96%, 97%, 98%, 99%, or 100% identical to the amino acid sequence of SEQ ID NO:5 (clade 1b);
(b) an amino acid sequence at least 95%, 96%, 97%, 98%, 99%, or 100% identical to the amino acid sequence of SEQ ID NO:13 (clade 1a); and
(c) an amino acid sequence at least 95%, 96%, 97%, 98%, 99%, or 100% identical to the amino acid sequence of SEQ ID NO:16 or 17 (Clade 3);
wherein the N-terminal methionine residue of SEQ ID NO:1 is optional and may be present or absent.

23. The nanoparticle of claim 1, wherein the nanoparticle comprises a plurality of fusion proteins, wherein the plurality of fusion proteins comprise a first domain comprising the amino acid sequence of SEQ ID NO:1, fused to:
(a) an amino acid sequence at least 98%, 99%, or 100% identical to the amino acid sequence of SEQ ID NO:5 (clade 1b);
(b) an amino acid sequence at least 98%, 99%, or 100% identical to the amino acid sequence of SEQ ID NO:13 (clade 1a); and
(c) an amino acid sequence at least 98%, 99%, or 100% identical to the amino acid sequence of SEQ ID NO:16 or 17 (Clade 3);
wherein the N-terminal methionine residue of SEQ ID NO:1 is optional and may be present or absent.

24. The nanoparticle of claim 1, wherein the nanoparticle comprises a plurality of fusion proteins, wherein the plurality of fusion proteins comprise a first domain comprising the amino acid sequence of SEQ ID NO:1, fused to:
(a) an amino acid sequence at least 99% or 100% identical to the amino acid sequence of SEQ ID NO:5 (clade 1b);
(b) an amino acid sequence at least 99% or 100% identical to the amino acid sequence of SEQ ID NO:13 (clade 1a); and
(c) an amino acid sequence at least 99% or 100% identical to the amino acid sequence of SEQ ID NO:16 or 17 (Clade 3);
wherein the N-terminal methionine residue of SEQ ID NO:1 is optional and may be present or absent.

25. The nanoparticle of claim 1, wherein the nanoparticle comprises a plurality of fusion proteins, wherein the plurality of fusion proteins comprise a first domain comprising an amino acid sequence at least 99% or 100% identical to the amino acid sequence of SEQ ID NO:1, fused to:
(a) an amino acid sequence at least 99% or 100% identical to the amino acid sequence of SEQ ID NO:5 (clade 1b);
(b) an amino acid sequence at least 99% or 100% identical to the amino acid sequence of SEQ ID NO:13 (clade 1a); and
(c) an amino acid sequence at least 99% or 100% identical to the amino acid sequence of SEQ ID NO:16 or 17 (Clade 3);
wherein the N-terminal methionine residue of SEQ ID NO:1 is optional and may be present or absent.

26. The nanoparticle of claim 1, wherein the nanoparticle comprises a plurality of fusion proteins, wherein the plurality of fusion proteins comprise a first domain comprising the amino acid sequence of SEQ ID NO:1, fused to:
(a) the amino acid sequence of SEQ ID NO:5 (clade 1b);
(b) the amino acid sequence of SEQ ID NO:13 (clade 1a); and
(c) the amino acid sequence of SEQ ID NO:16 or 17 (Clade 3);
wherein the N-terminal methionine residue of SEQ ID NO:1 is optional and may be present or absent.

27.-33. (canceled)

34. The nanoparticle of claim 1, wherein the nanoparticle comprises a plurality of fusion proteins, wherein the plurality of fusion proteins comprise a first domain comprising the amino acid sequence of SEQ ID NO:1, fused to:
(a) an amino acid sequence at least 80%, 85%, 90%, 91%, 92%, 93%, 94%, 95%, 96%, 97%, 98%, 99%, or 100% identical to the amino acid sequence of SEQ ID NO:5 (clade 1b);
(b) an amino acid sequence at least 80%, 85%, 90%, 91%, 92%, 93%, 94%, 95%, 96%, 97%, 98%, 99%, or 100% identical to the amino acid sequence of SEQ ID NO:13 (clade 1a);
(c) an amino acid sequence at least 80%, 85%, 90%, 91%, 92%, 93%, 94%, 95%, 96%, 97%, 98%, 99%, or 100% identical to the amino acid sequence of SEQ ID NO:16 or 17 (Clade 3); and
(d) an amino acid sequence at least 80%, 85%, 90%, 91%, 92%, 93%, 94%, 95%, 96%, 97%, 98%, 99%, or 100% identical to the amino acid sequence of SEQ ID NO:2 (clade 1b);
wherein the N-terminal methionine residue of SEQ ID NO:1 is optional and may be present or absent.

35.-54. (canceled)

55. A composition comprising a plurality of nanoparticles, wherein the plurality of nanoparticles comprise:
(a) a first nanoparticle that comprises a fusion protein comprising a first domain comprising an amino acid sequence at least 95%, 96%, 97%, 98%, 99%, or 100% identical to the amino acid sequence of SEQ ID NO:1, fused to an amino acid sequence at least 90%, 91%, 92%, 93%, 94%, 95%, 96%, 97%, 98%, 99%, or 100% identical to the amino acid sequence of SEQ ID NO:5 (clade 1b);
(b) a second nanoparticle that comprises a fusion protein comprising a first domain comprising an amino acid sequence at least 95%, 96%, 97%, 98%, 99%, or 100% identical to the amino acid sequence of SEQ ID NO:1, fused to an amino acid sequence at least 90%, 91%, 92%, 93%, 94%, 95%, 96%, 97%, 98%, 99%, or 100% identical to the amino acid sequence of SEQ ID NO: 13 (clade 1a); and
(c) a third nanoparticle that comprises a fusion protein comprising a first domain comprising an amino acid sequence at least 95%, 96%, 97%, 98%, 99%, or 100% identical to the amino acid sequence of SEQ ID NO:1, fused to an amino acid sequence at least 90%, 91%, 92%, 93%, 94%, 95%, 96%, 97%, 98%, 99%, or 100% identical to the amino acid sequence of SEQ ID NO:16 or 17 (Clade 3);
wherein the N-terminal methionine residue of SEQ ID NO:1 is optional and may be present or absent.

56.-87. (canceled)

88. A nucleic acid encoding the fusion protein recited in claim 1.

89.-96. (canceled)

97. A vaccine comprising the nanoparticles of claim 1.

98. A method to treat or limit development of a sarbecovirus infection, comprising administering to a subject in need thereof an amount effective to treat or limit development of the infection of the nanoparticles of claim 1.

99.-106. (canceled)
